SEQUENCE LISTING (600 sequences)

SEQ ID NO: 1 - protein, 11 aa, Artificial Sequence
  Synthetic peptide
AAVKNWMTQTL

SEQ ID NO: 2 - protein, 9 aa, Artificial Sequence
  Synthetic peptide
AGPHNDMEI

SEQ ID NO: 3 - protein, 9 aa, Artificial Sequence
  Synthetic peptide
AGVDNRECI

SEQ ID NO: 4 - protein, 9 aa, Artificial Sequence
  Synthetic peptide
AIQGNVTSI

SEQ ID NO: 5 - protein, 9 aa, Artificial Sequence
  Synthetic peptide
AQLANDVVL

SEQ ID NO: 6 - protein, 9 aa, Artificial Sequence
  Synthetic peptide
ASFRNLTHL

SEQ ID NO: 7 - protein, 9 aa, Artificial Sequence
  Synthetic peptide
ASMTNMELM

SEQ ID NO: 8 - protein, 9 aa, Artificial Sequence
  Synthetic peptide
ASNENMDAM

SEQ ID NO: 9 - protein, 9 aa, Artificial Sequence
  Synthetic peptide
ASNENMETM

SEQ ID NO: 10 - protein, 9 aa, Artificial Sequence
  Synthetic peptide
ATFKNWPFL

SEQ ID NO: 11 - protein, 9 aa, Artificial Sequence
  Synthetic peptide
CMTWNQMNL

SEQ ID NO: 12 - protein, 10 aa, Artificial Sequence
  Synthetic peptide
CSANNSHHYI

SEQ ID NO: 13 - protein, 9 aa, Artificial Sequence
  Synthetic peptide
FQPQNGQFI

SEQ ID NO: 14 - protein, 10 aa, Artificial Sequence
  Synthetic peptide
FSNSTNDILI

SEQ ID NO: 15 - protein, 9 aa, Artificial Sequence
  Synthetic peptide
GAVQNEVTL

SEQ ID NO: 16 - protein, 9 aa, Artificial Sequence
  Synthetic peptide
HCIRNKSVI

SEQ ID NO: 17 - protein, 10 aa, Artificial Sequence
  Synthetic peptide
HCIRNKSVIL

SEQ ID NO: 18 - protein, 9 aa, Artificial Sequence
  Synthetic peptide
HGIRNASFI

SEQ ID NO: 19 - protein, 9 aa, Artificial Sequence
  Synthetic peptide
KAVYNFATC

SEQ ID NO: 20 - protein, 9 aa, Artificial Sequence
  Synthetic peptide
KAVYNFATM

SEQ ID NO: 21 - protein, 9 aa, Artificial Sequence
  Synthetic peptide
KCSRNRQYL

SEQ ID NO: 22 - protein, 9 aa, Artificial Sequence
  Synthetic peptide
KVPRNQDWL

SEQ ID NO: 23 - protein, 9 aa, Artificial Sequence
  Synthetic peptide
LGMSNRDFL

SEQ ID NO: 24 - protein, 9 aa, Artificial Sequence
  Synthetic peptide
RAHYNIVTF

SEQ ID NO: 25 - protein, 9 aa, Artificial Sequence
  Synthetic peptide
RMFPNAPYL

SEQ ID NO: 26 - protein, 10 aa, Artificial Sequence
  Synthetic peptide
RSPFSRVVHL

SEQ ID NO: 27 - protein, 10 aa, Artificial Sequence
  Synthetic peptide
SCLENFRAYV

SEQ ID NO: 28 - protein, 10 aa, Artificial Sequence
  Synthetic peptide
SGPSNTPPEI

SEQ ID NO: 29 - protein, 11 aa, Artificial Sequence
  Synthetic peptide
SGVENPGGYCL

SEQ ID NO: 30 - protein, 9 aa, Artificial Sequence
  Synthetic peptide
SHLVEALYL

SEQ ID NO: 31 - protein, 9 aa, Artificial Sequence
  Synthetic peptide
SQLLNAKYL

SEQ ID NO: 32 - protein, 10 aa, Artificial Sequence
  Synthetic peptide
SSLENFRAYV

SEQ ID NO: 33 - protein, 9 aa, Artificial Sequence
  Synthetic peptide
VILTNPISM

SEQ ID NO: 34 - protein, 9 aa, Artificial Sequence
  Synthetic peptide
WMHHNMDLI

SEQ ID NO: 35 - protein, 9 aa, Artificial Sequence
  Synthetic peptide
AGPHNDMEI

SEQ ID NO: 36 - protein, 9 aa, Artificial Sequence
  Synthetic peptide
AGPPRYSRI

SEQ ID NO: 37 - protein, 9 aa, Artificial Sequence
  Synthetic peptide
IGPGRAFYA

SEQ ID NO: 38 - protein, 8 aa, Artificial Sequence
  Synthetic peptide
IPGRAFYA

SEQ ID NO: 39 - protein, 9 aa, Artificial Sequence
  Synthetic peptide
LGPISGHVL

SEQ ID NO: 40 - protein, 10 aa, Artificial Sequence
  Synthetic peptide
RGPGRAFVTI

SEQ ID NO: 41 - protein, 9 aa, Artificial Sequence
  Synthetic peptide
RRLGRTLLL

SEQ ID NO: 42 - protein, 8 aa, Artificial Sequence
  Synthetic peptide
AGLAYYSM

SEQ ID NO: 43 - protein, 8 aa, Artificial Sequence
  Synthetic peptide
ANYNFTLV

SEQ ID NO: 44 - protein, 8 aa, Artificial Sequence
  Synthetic peptide
ATLTYRML

SEQ ID NO: 45 - protein, 8 aa, Artificial Sequence
  Synthetic peptide
DAPIYTNV

SEQ ID NO: 46 - protein, 10 aa, Artificial Sequence
  Synthetic peptide
EVYDFAFRDL

SEQ ID NO: 47 - protein, 9 aa, Artificial Sequence
  Synthetic peptide
FAPGNYPAL

SEQ ID NO: 48 - protein, 8 aa, Artificial Sequence
  Synthetic peptide
HILIYSDV

SEQ ID NO: 49 - protein, 8 aa, Artificial Sequence
  Synthetic peptide
HNTQYCNL

SEQ ID NO: 50 - protein, 8 aa, Artificial Sequence
  Synthetic peptide
ICPMYARV

SEQ ID NO: 51 - protein, 9 aa, Artificial Sequence
  Synthetic peptide
ILSPFLPLL

SEQ ID NO: 52 - protein, 8 aa, Artificial Sequence
  Synthetic peptide
IMYNYPAM

SEQ ID NO: 53 - protein, 9 aa, Artificial Sequence
  Synthetic peptide
IMYNYPAML

SEQ ID NO: 54 - protein, 8 aa, Artificial Sequence
  Synthetic peptide
ISHNFCNL

SEQ ID NO: 55 - protein, 8 aa, Artificial Sequence
  Synthetic peptide
KSPWFTTL

SEQ ID NO: 56 - protein, 8 aa, Artificial Sequence
  Synthetic peptide
KVVRFDKL

SEQ ID NO: 57 - protein, 8 aa, Artificial Sequence
  Synthetic peptide
LTFNYRNL

SEQ ID NO: 58 - protein, 8 aa, Artificial Sequence
  Synthetic peptide
MGLKFRQL

SEQ ID NO: 59 - protein, 8 aa, Artificial Sequence
  Synthetic peptide
RGYVYQGL

SEQ ID NO: 60 - protein, 9 aa, Artificial Sequence
  Synthetic peptide
SCLEFWQRV

SEQ ID NO: 61 - protein, 8 aa, Artificial Sequence
  Synthetic peptide
SDYYFSWL

SEQ ID NO: 62 - protein, 8 aa, Artificial Sequence
  Synthetic peptide
SIINFEKL

SEQ ID NO: 63 - protein, 8 aa, Artificial Sequence
  Synthetic peptide
SIIVFNLL

SEQ ID NO: 64 - protein, 8 aa, Artificial Sequence
  Synthetic peptide
SIYRYYGL

SEQ ID NO: 65 - protein, 8 aa, Artificial Sequence
  Synthetic peptide
SKYVFENV

SEQ ID NO: 66 - protein, 8 aa, Artificial Sequence
  Synthetic peptide
SSIEFARL

SEQ ID NO: 67 - protein, 8 aa, Artificial Sequence
  Synthetic peptide
STYTFVRT

SEQ ID NO: 68 - protein, 8 aa, Artificial Sequence
  Synthetic peptide
SVLAFRRL

SEQ ID NO: 69 - protein, 9 aa, Artificial Sequence
  Synthetic peptide
SVYDFFVWL

SEQ ID NO: 70 - protein, 8 aa, Artificial Sequence
  Synthetic peptide
TSINFVKI

SEQ ID NO: 71 - protein, 8 aa, Artificial Sequence
  Synthetic peptide
TSYKFESV

SEQ ID NO: 72 - protein, 8 aa, Artificial Sequence
  Synthetic peptide
TVSEFLKL

SEQ ID NO: 73 - protein, 8 aa, Artificial Sequence
  Synthetic peptide
TVYGFCLL

SEQ ID NO: 74 - protein, 8 aa, Artificial Sequence
  Synthetic peptide
VGRNFTNL

SEQ ID NO: 75 - protein, 8 aa, Artificial Sequence
  Synthetic peptide
VIDAFSRL

SEQ ID NO: 76 - protein, 9 aa, Artificial Sequence
  Synthetic peptide
VIYIFTVRL

SEQ ID NO: 77 - protein, 8 aa, Artificial Sequence
  Synthetic peptide
VNHRFTLV

SEQ ID NO: 78 - protein, 8 aa, Artificial Sequence
  Synthetic peptide
VVYDFLKL

SEQ ID NO: 79 - protein, 8 aa, Artificial Sequence
  Synthetic peptide
VWLSVIWM

SEQ ID NO: 80 - protein, 9 aa, Artificial Sequence
  Synthetic peptide
AMQMLKDTI

SEQ ID NO: 81 - protein, 9 aa, Artificial Sequence
  Synthetic peptide
AMQMLKETI

SEQ ID NO: 82 - protein, 9 aa, Artificial Sequence
  Synthetic peptide
AYIDFEMKI

SEQ ID NO: 83 - protein, 9 aa, Artificial Sequence
  Synthetic peptide
CYYASRTKL

SEQ ID NO: 84 - protein, 9 aa, Artificial Sequence
  Synthetic peptide
DYWGQGTEL

SEQ ID NO: 85 - protein, 9 aa, Artificial Sequence
  Synthetic peptide
EYILSLEEL

SEQ ID NO: 86 - protein, 9 aa, Artificial Sequence
  Synthetic peptide
GYETVITQL

SEQ ID NO: 87 - protein, 9 aa, Artificial Sequence
  Synthetic peptide
GYKDGNEYI

SEQ ID NO: 88 - protein, 9 aa, Artificial Sequence
  Synthetic peptide
HYLSTQSAL

SEQ ID NO: 89 - protein, 9 aa, Artificial Sequence
  Synthetic peptide
IYNVGQVSI

SEQ ID NO: 90 - protein, 9 aa, Artificial Sequence
  Synthetic peptide
IYSTVASSL

SEQ ID NO: 91 - protein, 9 aa, Artificial Sequence
  Synthetic peptide
KYKNAVTEL

SEQ ID NO: 92 - protein, 9 aa, Artificial Sequence
  Synthetic peptide
KYNKANVFL

SEQ ID NO: 93 - protein, 9 aa, Artificial Sequence
  Synthetic peptide
QYIHSANVL

SEQ ID NO: 94 - protein, 10 aa, Artificial Sequence
  Synthetic peptide
RYLKNGKETL

SEQ ID NO: 95 - protein, 9 aa, Artificial Sequence
  Synthetic peptide
SYIGSINNI

SEQ ID NO: 96 - protein, 9 aa, Artificial Sequence
  Synthetic peptide
SYIPSAEKI

SEQ ID NO: 97 - protein, 9 aa, Artificial Sequence
  Synthetic peptide
SYMLQALCI

SEQ ID NO: 98 - protein, 9 aa, Artificial Sequence
  Synthetic peptide
SYVPSAEQI

SEQ ID NO: 99 - protein, 9 aa, Artificial Sequence
  Synthetic peptide
TYLPTNASL

SEQ ID NO: 100 - protein, 9 aa, Artificial Sequence
  Synthetic peptide
TYQRTRALV

SEQ ID NO: 101 - protein, 9 aa, Artificial Sequence
  Synthetic peptide
TYVPANASL

SEQ ID NO: 102 - protein, 9 aa, Artificial Sequence
  Synthetic peptide
TYWPVVSDI

SEQ ID NO: 103 - protein, 9 aa, Artificial Sequence
  Synthetic peptide
VYAGAMSGL

SEQ ID NO: 104 - protein, 9 aa, Artificial Sequence
  Synthetic peptide
YYIPHQSSL

SEQ ID NO: 105 - protein, 10 aa, Artificial Sequence
  Synthetic peptide
DYENDIEKKI

SEQ ID NO: 106 - protein, 8 aa, Artificial Sequence
  Synthetic peptide
FETFEAKI

SEQ ID NO: 107 - protein, 8 aa, Artificial Sequence
  Synthetic peptide
TENSGKDI

SEQ ID NO: 108 - protein, 8 aa, Artificial Sequence
  Synthetic peptide
TEWETGQI

SEQ ID NO: 109 - protein, 8 aa, Artificial Sequence
  Synthetic peptide
VESTAGSL

SEQ ID NO: 110 - protein, 9 aa, Artificial Sequence
  Synthetic peptide
YENDIEKKI

SEQ ID NO: 111 - protein, 9 aa, Artificial Sequence
  Synthetic peptide
HPQKVTKFM

SEQ ID NO: 112 - protein, 12 aa, Artificial Sequence
  Synthetic peptide
IPQSLDSWWTSL

SEQ ID NO: 113 - protein, 9 aa, Artificial Sequence
  Synthetic peptide
LPYLGWLVF

SEQ ID NO: 114 - protein, 9 aa, Artificial Sequence
  Synthetic peptide
MPVGGQSSF

SEQ ID NO: 115 - protein, 9 aa, Artificial Sequence
  Synthetic peptide
MPYLIDFGL

SEQ ID NO: 116 - protein, 9 aa, Artificial Sequence
  Synthetic peptide
RPQASGVYM

SEQ ID NO: 117 - protein, 9 aa, Artificial Sequence
  Synthetic peptide
SPGAAGYDL

SEQ ID NO: 118 - protein, 9 aa, Artificial Sequence
  Synthetic peptide
SPSYVYHQF

SEQ ID NO: 119 - protein, 9 aa, Artificial Sequence
  Synthetic peptide
TPHPARIGL

SEQ ID NO: 120 - protein, 9 aa, Artificial Sequence
  Synthetic peptide
YPHFMPTNL

SEQ ID NO: 121 - protein, 9 aa, Artificial Sequence
  Synthetic peptide
ATDALMTGF

SEQ ID NO: 122 - protein, 9 aa, Artificial Sequence
  Synthetic peptide
ATDALMTGY

SEQ ID NO: 123 - protein, 9 aa, Artificial Sequence
  Synthetic peptide
CTELKLSDY

SEQ ID NO: 124 - protein, 9 aa, Artificial Sequence
  Synthetic peptide
EADPTGHSY

SEQ ID NO: 125 - protein, 9 aa, Artificial Sequence
  Synthetic peptide
EVDPIGHLY

SEQ ID NO: 126 - protein, 9 aa, Artificial Sequence
  Synthetic peptide
FTELTLGEF

SEQ ID NO: 127 - protein, 9 aa, Artificial Sequence
  Synthetic peptide
IVDCLTEMY

SEQ ID NO: 128 - protein, 9 aa, Artificial Sequence
  Synthetic peptide
KSDICTDEY

SEQ ID NO: 129 - protein, 9 aa, Artificial Sequence
  Synthetic peptide
SADNNNSEY

SEQ ID NO: 130 - protein, 11 aa, Artificial Sequence
  Synthetic peptide
SSDYVIPIGTY

SEQ ID NO: 131 - protein, 9 aa, Artificial Sequence
  Synthetic peptide
TDLGQNLLY

SEQ ID NO: 132 - protein, 9 aa, Artificial Sequence
  Synthetic peptide
TLDTLTAFY

SEQ ID NO: 133 - protein, 11 aa, Artificial Sequence
  Synthetic peptide
TSEKRPFMCAY

SEQ ID NO: 134 - protein, 9 aa, Artificial Sequence
  Synthetic peptide
VSDGGPNLY

SEQ ID NO: 135 - protein, 9 aa, Artificial Sequence
  Synthetic peptide
VTEHDTLLY

SEQ ID NO: 136 - protein, 11 aa, Artificial Sequence
  Synthetic peptide
YSEHPTFTSQY

SEQ ID NO: 137 - protein, 9 aa, Artificial Sequence
  Synthetic peptide
AILALLPAL

SEQ ID NO: 138 - protein, 9 aa, Artificial Sequence
  Synthetic peptide
AIQDLCLAV

SEQ ID NO: 139 - protein, 9 aa, Artificial Sequence
  Synthetic peptide
AIQDLCVAV

SEQ ID NO: 140 - protein, 9 aa, Artificial Sequence
  Synthetic peptide
AITEVECFL

SEQ ID NO: 141 - protein, 9 aa, Artificial Sequence
  Synthetic peptide
ALCNTDSPL

SEQ ID NO: 142 - protein, 9 aa, Artificial Sequence
  Synthetic peptide
ALDVYNGLL

SEQ ID NO: 143 - protein, 9 aa, Artificial Sequence
  Synthetic peptide
ALFDIESKV

SEQ ID NO: 144 - protein, 9 aa, Artificial Sequence
  Synthetic peptide
ALIAPVHAV

SEQ ID NO: 145 - protein, 9 aa, Artificial Sequence
  Synthetic peptide
ALISAFSGS

SEQ ID NO: 146 - protein, 9 aa, Artificial Sequence
  Synthetic peptide
ALKDVEERV

SEQ ID NO: 147 - protein, 9 aa, Artificial Sequence
  Synthetic peptide
ALLEIASCL

SEQ ID NO: 148 - protein, 10 aa, Artificial Sequence
  Synthetic peptide
ALLTSRLRFI

SEQ ID NO: 149 - protein, 9 aa, Artificial Sequence
  Synthetic peptide
ALMEQQHYV

SEQ ID NO: 150 - protein, 9 aa, Artificial Sequence
  Synthetic peptide
ALNVYNGLL

SEQ ID NO: 151 - protein, 9 aa, Artificial Sequence
  Synthetic peptide
ALPFGFILV

SEQ ID NO: 152 - protein, 9 aa, Artificial Sequence
  Synthetic peptide
ALPHIIDEV

SEQ ID NO: 153 - protein, 9 aa, Artificial Sequence
  Synthetic peptide
ALQPGTALL

SEQ ID NO: 154 - protein, 9 aa, Artificial Sequence
  Synthetic peptide
ALSPVPPVV

SEQ ID NO: 155 - protein, 9 aa, Artificial Sequence
  Synthetic peptide
ALTPVVVTL

SEQ ID NO: 156 - protein, 9 aa, Artificial Sequence
  Synthetic peptide
ALVCYGPGI

SEQ ID NO: 157 - protein, 9 aa, Artificial Sequence
  Synthetic peptide
ALVEMGHHA

SEQ ID NO: 158 - protein, 9 aa, Artificial Sequence
  Synthetic peptide
ALWALPHAA

SEQ ID NO: 159 - protein, 10 aa, Artificial Sequence
  Synthetic peptide
ALWGPDPAAA

SEQ ID NO: 160 - protein, 10 aa, Artificial Sequence
  Synthetic peptide
ALWPWLLMAT

SEQ ID NO: 161 - protein, 9 aa, Artificial Sequence
  Synthetic peptide
ALYDVVTKL

SEQ ID NO: 162 - protein, 9 aa, Artificial Sequence
  Synthetic peptide
ALYLMELTM

SEQ ID NO: 163 - protein, 10 aa, Artificial Sequence
  Synthetic peptide
ALYVDSLFFL

SEQ ID NO: 164 - protein, 9 aa, Artificial Sequence
  Synthetic peptide
AMASTEGNV

SEQ ID NO: 165 - protein, 9 aa, Artificial Sequence
  Synthetic peptide
AMLVLLAEI

SEQ ID NO: 166 - protein, 9 aa, Artificial Sequence
  Synthetic peptide
AQCQETIRV

SEQ ID NO: 167 - protein, 9 aa, Artificial Sequence
  Synthetic peptide
ATGEALWAL

SEQ ID NO: 168 - protein, 9 aa, Artificial Sequence
  Synthetic peptide
ATWAENIQV

SEQ ID NO: 169 - protein, 9 aa, Artificial Sequence
  Synthetic peptide
AVLDGLLSL

SEQ ID NO: 170 - protein, 9 aa, Artificial Sequence
  Synthetic peptide
CINGVCWTV

SEQ ID NO: 171 - protein, 9 aa, Artificial Sequence
  Synthetic peptide
CLGGLLTMV

SEQ ID NO: 172 - protein, 9 aa, Artificial Sequence
  Synthetic peptide
CLPSPSTPV

SEQ ID NO: 173 - protein, 9 aa, Artificial Sequence
  Synthetic peptide
CLWCVPQLR

SEQ ID NO: 174 - protein, 9 aa, Artificial Sequence
  Synthetic peptide
CVNGVCWTV

SEQ ID NO: 175 - protein, 9 aa, Artificial Sequence
  Synthetic peptide
DIWDGIPHV

SEQ ID NO: 176 - protein, 9 aa, Artificial Sequence
  Synthetic peptide
DLMGYIPAV

SEQ ID NO: 177 - protein, 9 aa, Artificial Sequence
  Synthetic peptide
DLMGYIPLV

SEQ ID NO: 178 - protein, 9 aa, Artificial Sequence
  Synthetic peptide
EIWTHSYKV

SEQ ID NO: 179 - protein, 10 aa, Artificial Sequence
  Synthetic peptide
ELAGIGILTV

SEQ ID NO: 180 - protein, 9 aa, Artificial Sequence
  Synthetic peptide
ELSDSLGPV

SEQ ID NO: 181 - protein, 10 aa, Artificial Sequence
  Synthetic peptide
ELTLGEFLKL

SEQ ID NO: 182 - protein, 9 aa, Artificial Sequence
  Synthetic peptide
ELVDGLLSL

SEQ ID NO: 183 - protein, 9 aa, Artificial Sequence
  Synthetic peptide
FIDSYICQV

SEQ ID NO: 184 - protein, 9 aa, Artificial Sequence
  Synthetic peptide
FILGIIITV

SEQ ID NO: 185 - protein, 10 aa, Artificial Sequence
  Synthetic peptide
FIYDFCIFGV

SEQ ID NO: 186 - protein, 10 aa, Artificial Sequence
  Synthetic peptide
FLAEDALNTV

SEQ ID NO: 187 - protein, 9 aa, Artificial Sequence
  Synthetic peptide
FLAMLKNTV

SEQ ID NO: 188 - protein, 9 aa, Artificial Sequence
  Synthetic peptide
FLDKGTYTL

SEQ ID NO: 189 - protein, 9 aa, Artificial Sequence
  Synthetic peptide
FLDPRPLTV

SEQ ID NO: 190 - protein, 9 aa, Artificial Sequence
  Synthetic peptide
FLFLRNFSL

SEQ ID NO: 191 - protein, 8 aa, Artificial Sequence
  Synthetic peptide
FLGKIWPS

SEQ ID NO: 192 - protein, 9 aa, Artificial Sequence
  Synthetic peptide
FLGYLILGV

SEQ ID NO: 193 - protein, 10 aa, Artificial Sequence
  Synthetic peptide
FLLSLFSLWL

SEQ ID NO: 194 - protein, 9 aa, Artificial Sequence
  Synthetic peptide
FLLSLGIHL

SEQ ID NO: 195 - protein, 9 aa, Artificial Sequence
  Synthetic peptide
FLLTRILTI

SEQ ID NO: 196 - protein, 9 aa, Artificial Sequence
  Synthetic peptide
FLNKCETWV

SEQ ID NO: 197 - protein, 10 aa, Artificial Sequence
  Synthetic peptide
FLPSDFFPSI

SEQ ID NO: 198 - protein, 10 aa, Artificial Sequence
  Synthetic peptide
FLPSDFFPSV

SEQ ID NO: 199 - protein, 10 aa, Artificial Sequence
  Synthetic peptide
FLPSPLFFFL

SEQ ID NO: 200 - protein, 10 aa, Artificial Sequence
  Synthetic peptide
FLTPKKLQCV

SEQ ID NO: 201 - protein, 9 aa, Artificial Sequence
  Synthetic peptide
FLWGPRALV

SEQ ID NO: 202 - protein, 9 aa, Artificial Sequence
  Synthetic peptide
FLYALALLL

SEQ ID NO: 203 - protein, 9 aa, Artificial Sequence
  Synthetic peptide
FLYDDNQRV

SEQ ID NO: 204 - protein, 9 aa, Artificial Sequence
  Synthetic peptide
FMNKFIYEI

SEQ ID NO: 205 - protein, 9 aa, Artificial Sequence
  Synthetic peptide
FVGEFFTDV

SEQ ID NO: 206 - protein, 9 aa, Artificial Sequence
  Synthetic peptide
GILGFVFTL

SEQ ID NO: 207 - protein, 9 aa, Artificial Sequence
  Synthetic peptide
GILTVSVAV

SEQ ID NO: 208 - protein, 9 aa, Artificial Sequence
  Synthetic peptide
GLADQLIHL

SEQ ID NO: 209 - protein, 11 aa, Artificial Sequence
  Synthetic peptide
GLAPPQHLIRV

SEQ ID NO: 210 - protein, 9 aa, Artificial Sequence
  Synthetic peptide
GLCTLVAML

SEQ ID NO: 211 - protein, 9 aa, Artificial Sequence
  Synthetic peptide
GLFKCGIAV

SEQ ID NO: 212 - protein, 9 aa, Artificial Sequence
  Synthetic peptide
GLIQLVEGV

SEQ ID NO: 213 - protein, 10 aa, Artificial Sequence
  Synthetic peptide
GLLGASVLGL

SEQ ID NO: 214 - protein, 9 aa, Artificial Sequence
  Synthetic peptide
GLLRFVTAV

SEQ ID NO: 215 - protein, 9 aa, Artificial Sequence
  Synthetic peptide
GLLSLEEEL

SEQ ID NO: 216 - protein, 9 aa, Artificial Sequence
  Synthetic peptide
GLMEEMSAL

SEQ ID NO: 217 - protein, 9 aa, Artificial Sequence
  Synthetic peptide
GLPVEYLQV

SEQ ID NO: 218 - protein, 9 aa, Artificial Sequence
  Synthetic peptide
GLQDCTMLV

SEQ ID NO: 219 - protein, 9 aa, Artificial Sequence
  Synthetic peptide
GLQHWVPEL

SEQ ID NO: 220 - protein, 10 aa, Artificial Sequence
  Synthetic peptide
GLSPTVWLSV

SEQ ID NO: 221 - protein, 9 aa, Artificial Sequence
  Synthetic peptide
GLSRYVARL

SEQ ID NO: 222 - protein, 9 aa, Artificial Sequence
  Synthetic peptide
GLYDGMEHL

SEQ ID NO: 223 - protein, 9 aa, Artificial Sequence
  Synthetic peptide
GMLGMVSGL

SEQ ID NO: 224 - protein, 10 aa, Artificial Sequence
  Synthetic peptide
GVDPNIRTGV

SEQ ID NO: 225 - protein, 9 aa, Artificial Sequence
  Synthetic peptide
GVLVGVALI

SEQ ID NO: 226 - protein, 9 aa, Artificial Sequence
  Synthetic peptide
GVRGRVEEI

SEQ ID NO: 227 - protein, 10 aa, Artificial Sequence
  Synthetic peptide
GVYDGREHTV

SEQ ID NO: 228 - protein, 9 aa, Artificial Sequence
  Synthetic peptide
HIAGSLAVV

SEQ ID NO: 229 - protein, 9 aa, Artificial Sequence
  Synthetic peptide
HLSTAFARV

SEQ ID NO: 230 - protein, 9 aa, Artificial Sequence
  Synthetic peptide
HLVEALYLV

SEQ ID NO: 231 - protein, 9 aa, Artificial Sequence
  Synthetic peptide
ILAKFLHWL

SEQ ID NO: 232 - protein, 9 aa, Artificial Sequence
  Synthetic peptide
ILDDNLYKV

SEQ ID NO: 233 - protein, 10 aa, Artificial Sequence
  Synthetic peptide
ILGFVFTLTV

SEQ ID NO: 234 - protein, 9 aa, Artificial Sequence
  Synthetic peptide
ILGVLTSLV

SEQ ID NO: 235 - protein, 9 aa, Artificial Sequence
  Synthetic peptide
ILHDGAYSL

SEQ ID NO: 236 - protein, 9 aa, Artificial Sequence
  Synthetic peptide
ILHNGAYSL

SEQ ID NO: 237 - protein, 9 aa, Artificial Sequence
  Synthetic peptide
ILKDFSILL

SEQ ID NO: 238 - protein, 9 aa, Artificial Sequence
  Synthetic peptide
ILKEPVHGV

SEQ ID NO: 239 - protein, 9 aa, Artificial Sequence
  Synthetic peptide
ILLWEIFTL

SEQ ID NO: 240 - protein, 9 aa, Artificial Sequence
  Synthetic peptide
ILLWQPIPV

SEQ ID NO: 241 - protein, 9 aa, Artificial Sequence
  Synthetic peptide
ILMWEAVTL

SEQ ID NO: 242 - protein, 9 aa, Artificial Sequence
  Synthetic peptide
ILSLELMKL

SEQ ID NO: 243 - protein, 9 aa, Artificial Sequence
  Synthetic peptide
IMDQVPFSV

SEQ ID NO: 244 - protein, 9 aa, Artificial Sequence
  Synthetic peptide
ITDQVPFSV

SEQ ID NO: 245 - protein, 9 aa, Artificial Sequence
  Synthetic peptide
KIFGSLAFL

SEQ ID NO: 246 - protein, 9 aa, Artificial Sequence
  Synthetic peptide
KLCPVQLWV

SEQ ID NO: 247 - protein, 9 aa, Artificial Sequence
  Synthetic peptide
KLDVGNAEV

SEQ ID NO: 248 - protein, 10 aa, Artificial Sequence
  Synthetic peptide
KLFGTSGQKT

SEQ ID NO: 249 - protein, 10 aa, Artificial Sequence
  Synthetic peptide
KLGEFYNQMM

SEQ ID NO: 250 - protein, 9 aa, Artificial Sequence
  Synthetic peptide
KLHLYSHPI

SEQ ID NO: 251 - protein, 9 aa, Artificial Sequence
  Synthetic peptide
KLIANNTRV

SEQ ID NO: 252 - protein, 10 aa, Artificial Sequence
  Synthetic peptide
KLMSSNSTDL

SEQ ID NO: 253 - protein, 9 aa, Artificial Sequence
  Synthetic peptide
KLPQLCTEL

SEQ ID NO: 254 - protein, 9 aa, Artificial Sequence
  Synthetic peptide
KLQCVDLHV

SEQ ID NO: 255 - protein, 9 aa, Artificial Sequence
  Synthetic peptide
KLQDASAEV

SEQ ID NO: 256 - protein, 9 aa, Artificial Sequence
  Synthetic peptide
KLQVFLIVL

SEQ ID NO: 257 - protein, 10 aa, Artificial Sequence
  Synthetic peptide
KLSGLGINAV

SEQ ID NO: 258 - protein, 9 aa, Artificial Sequence
  Synthetic peptide
KLTPLCVTL

SEQ ID NO: 259 - protein, 10 aa, Artificial Sequence
  Synthetic peptide
KLVALGINAV

SEQ ID NO: 260 - protein, 9 aa, Artificial Sequence
  Synthetic peptide
KMLKEMGEV

SEQ ID NO: 261 - protein, 9 aa, Artificial Sequence
  Synthetic peptide
KTWGQYWQV

SEQ ID NO: 262 - protein, 10 aa, Artificial Sequence
  Synthetic peptide
KVAEELVHFL

SEQ ID NO: 263 - protein, 9 aa, Artificial Sequence
  Synthetic peptide
KVAELVHFL

SEQ ID NO: 264 - protein, 9 aa, Artificial Sequence
  Synthetic peptide
KVDDTFYYV

SEQ ID NO: 265 - protein, 9 aa, Artificial Sequence
  Synthetic peptide
KVLEYVIKV

SEQ ID NO: 266 - protein, 9 aa, Artificial Sequence
  Synthetic peptide
KVVEFLAML

SEQ ID NO: 267 - protein, 9 aa, Artificial Sequence
  Synthetic peptide
LAALPHSCL

SEQ ID NO: 268 - protein, 10 aa, Artificial Sequence
  Synthetic peptide
LIAHNQVRQV

SEQ ID NO: 269 - protein, 9 aa, Artificial Sequence
  Synthetic peptide
LIDQYLYYL

SEQ ID NO: 270 - protein, 9 aa, Artificial Sequence
  Synthetic peptide
LILPLLFYL

SEQ ID NO: 271 - protein, 10 aa, Artificial Sequence
  Synthetic peptide
LLAARAIVAI

SEQ ID NO: 272 - protein, 10 aa, Artificial Sequence
  Synthetic peptide
LLDFVRFMGV

SEQ ID NO: 273 - protein, 9 aa, Artificial Sequence
  Synthetic peptide
LLDVPTAAV

SEQ ID NO: 274 - protein, 10 aa, Artificial Sequence
  Synthetic peptide
LLFGLALIEV

SEQ ID NO: 275 - protein, 9 aa, Artificial Sequence
  Synthetic peptide
LLFGYPVYV

SEQ ID NO: 276 - protein, 10 aa, Artificial Sequence
  Synthetic peptide
LLFNILGGWV

SEQ ID NO: 277 - protein, 9 aa, Artificial Sequence
  Synthetic peptide
LLGRNSFEV

SEQ ID NO: 278 - protein, 9 aa, Artificial Sequence
  Synthetic peptide
LLHETDSAV

SEQ ID NO: 279 - protein, 10 aa, Artificial Sequence
  Synthetic peptide
LLLASIAAGL

SEQ ID NO: 280 - protein, 9 aa, Artificial Sequence
  Synthetic peptide
LLLGPLGPL

SEQ ID NO: 281 - protein, 9 aa, Artificial Sequence
  Synthetic peptide
LLLIWFRPV

SEQ ID NO: 282 - protein, 9 aa, Artificial Sequence
  Synthetic peptide
LLLLTVLTV

SEQ ID NO: 283 - protein, 9 aa, Artificial Sequence
  Synthetic peptide
LLLNCLWSV

SEQ ID NO: 284 - protein, 9 aa, Artificial Sequence
  Synthetic peptide
LLLTVLTVV

SEQ ID NO: 285 - protein, 10 aa, Artificial Sequence
  Synthetic peptide
LLMGTLGIVC

SEQ ID NO: 286 - protein, 9 aa, Artificial Sequence
  Synthetic peptide
LLMWEAVTV

SEQ ID NO: 287 - protein, 9 aa, Artificial Sequence
  Synthetic peptide
LLNGWRWRL

SEQ ID NO: 288 - protein, 10 aa, Artificial Sequence
  Synthetic peptide
LLQERGVAYI

SEQ ID NO: 289 - protein, 9 aa, Artificial Sequence
  Synthetic peptide
LLTAALWYV

SEQ ID NO: 290 - protein, 10 aa, Artificial Sequence
  Synthetic peptide
LLVPTCVFLV

SEQ ID NO: 291 - protein, 9 aa, Artificial Sequence
  Synthetic peptide
LLWNGPMAV

SEQ ID NO: 292 - protein, 9 aa, Artificial Sequence
  Synthetic peptide
LMLGEFLKL

SEQ ID NO: 293 - protein, 9 aa, Artificial Sequence
  Synthetic peptide
LMWYELSKI

SEQ ID NO: 294 - protein, 9 aa, Artificial Sequence
  Synthetic peptide
LNIDLLWSV

SEQ ID NO: 295 - protein, 9 aa, Artificial Sequence
  Synthetic peptide
LTFGWCFKL

SEQ ID NO: 296 - protein, 9 aa, Artificial Sequence
  Synthetic peptide
LTLGEFLKL

SEQ ID NO: 297 - protein, 9 aa, Artificial Sequence
  Synthetic peptide
LVWMACHSA

SEQ ID NO: 298 - protein, 9 aa, Artificial Sequence
  Synthetic peptide
MLAVFLPIV

SEQ ID NO: 299 - protein, 9 aa, Artificial Sequence
  Synthetic peptide
MLDLQPETT

SEQ ID NO: 300 - protein, 11 aa, Artificial Sequence
  Synthetic peptide
MLMAQEALAFL

SEQ ID NO: 301 - protein, 9 aa, Artificial Sequence
  Synthetic peptide
MLNIPSINV

SEQ ID NO: 302 - protein, 9 aa, Artificial Sequence
  Synthetic peptide
MMNDQLMFL

SEQ ID NO: 303 - protein, 9 aa, Artificial Sequence
  Synthetic peptide
MVWESGCTV

SEQ ID NO: 304 - protein, 9 aa, Artificial Sequence
  Synthetic peptide
NLFETPVEA

SEQ ID NO: 305 - protein, 9 aa, Artificial Sequence
  Synthetic peptide
NLVPMVATV

SEQ ID NO: 306 - protein, 9 aa, Artificial Sequence
  Synthetic peptide
NVWATHACV

SEQ ID NO: 307 - protein, 10 aa, Artificial Sequence
  Synthetic peptide
PLFDFSWLSL

SEQ ID NO: 308 - protein, 10 aa, Artificial Sequence
  Synthetic peptide
QLCPICRAPV

SEQ ID NO: 309 - protein, 9 aa, Artificial Sequence
  Synthetic peptide
QLFEELQEL

SEQ ID NO: 310 - protein, 9 aa, Artificial Sequence
  Synthetic peptide
QLFNHTMFI

SEQ ID NO: 311 - protein, 9 aa, Artificial Sequence
  Synthetic peptide
QLGEQCWTV

SEQ ID NO: 312 - protein, 10 aa, Artificial Sequence
  Synthetic peptide
QLLDGFMITL

SEQ ID NO: 313 - protein, 9 aa, Artificial Sequence
  Synthetic peptide
QLLIKAVNL

SEQ ID NO: 314 - protein, 9 aa, Artificial Sequence
  Synthetic peptide
QMARLAWEA

SEQ ID NO: 315 - protein, 9 aa, Artificial Sequence
  Synthetic peptide
QQAHCLWCV

SEQ ID NO: 316 - protein, 9 aa, Artificial Sequence
  Synthetic peptide
RILGAVAKV

SEQ ID NO: 317 - protein, 9 aa, Artificial Sequence
  Synthetic peptide
RLAEYQAYI

SEQ ID NO: 318 - protein, 10 aa, Artificial Sequence
  Synthetic peptide
RLDDDGNFQL

SEQ ID NO: 319 - protein, 10 aa, Artificial Sequence
  Synthetic peptide
RLLGNVLVCV

SEQ ID NO: 320 - protein, 9 aa, Artificial Sequence
  Synthetic peptide
RLLQETELV

SEQ ID NO: 321 - protein, 9 aa, Artificial Sequence
  Synthetic peptide
RLLVVYPWT

SEQ ID NO: 322 - protein, 9 aa, Artificial Sequence
  Synthetic peptide
RLMNDMTAV

SEQ ID NO: 323 - protein, 9 aa, Artificial Sequence
  Synthetic peptide
RLNMFTPYI

SEQ ID NO: 324 - protein, 9 aa, Artificial Sequence
  Synthetic peptide
RLSSCVPVA

SEQ ID NO: 325 - protein, 9 aa, Artificial Sequence
  Synthetic peptide
RLTPGVHEL

SEQ ID NO: 326 - protein, 9 aa, Artificial Sequence
  Synthetic peptide
RLTSRVKAL

SEQ ID NO: 327 - protein, 9 aa, Artificial Sequence
  Synthetic peptide
RLVDDFLLV

SEQ ID NO: 328 - protein, 9 aa, Artificial Sequence
  Synthetic peptide
RLWQELSDI

SEQ ID NO: 329 - protein, 9 aa, Artificial Sequence
  Synthetic peptide
RMFPNAPYL

SEQ ID NO: 330 - protein, 9 aa, Artificial Sequence
  Synthetic peptide
RMPEAAPPV

SEQ ID NO: 331 - protein, 9 aa, Artificial Sequence
  Synthetic peptide
RTLDKVLEV

SEQ ID NO: 332 - protein, 9 aa, Artificial Sequence
  Synthetic peptide
RTLNAWVKV

SEQ ID NO: 333 - protein, 9 aa, Artificial Sequence
  Synthetic peptide
RVASPTSGV

SEQ ID NO: 334 - protein, 9 aa, Artificial Sequence
  Synthetic peptide
SIDWFMVTV

SEQ ID NO: 335 - protein, 10 aa, Artificial Sequence
  Synthetic peptide
SILLRDAGLV

SEQ ID NO: 336 - protein, 9 aa, Artificial Sequence
  Synthetic peptide
SITEVECFL

SEQ ID NO: 337 - protein, 9 aa, Artificial Sequence
  Synthetic peptide
SLFEPPPPG

SEQ ID NO: 338 - protein, 9 aa, Artificial Sequence
  Synthetic peptide
SLFLGILSV

SEQ ID NO: 339 - protein, 9 aa, Artificial Sequence
  Synthetic peptide
SLFNTVATL

SEQ ID NO: 340 - protein, 10 aa, Artificial Sequence
  Synthetic peptide
SLFNTVATLY

SEQ ID NO: 341 - protein, 9 aa, Artificial Sequence
  Synthetic peptide
SLGEQQYSV

SEQ ID NO: 342 - protein, 9 aa, Artificial Sequence
  Synthetic peptide
SLLFLLFSL

SEQ ID NO: 343 - protein, 9 aa, Artificial Sequence
  Synthetic peptide
SLLMWITQC

SEQ ID NO: 344 - protein, 9 aa, Artificial Sequence
  Synthetic peptide
SLLMWITQV

SEQ ID NO: 345 - protein, 10 aa, Artificial Sequence
  Synthetic peptide
SLLNATAIAV

SEQ ID NO: 346 - protein, 9 aa, Artificial Sequence
  Synthetic peptide
SLLQHLIGL

SEQ ID NO: 347 - protein, 9 aa, Artificial Sequence
  Synthetic peptide
SLNQTVHSL

SEQ ID NO: 348 - protein, 9 aa, Artificial Sequence
  Synthetic peptide
SLPPPGTRV

SEQ ID NO: 349 - protein, 9 aa, Artificial Sequence
  Synthetic peptide
SLSEKTVLL

SEQ ID NO: 350 - protein, 9 aa, Artificial Sequence
  Synthetic peptide
SLSRFSWGA

SEQ ID NO: 351 - protein, 9 aa, Artificial Sequence
  Synthetic peptide
SLVDVMPWL

SEQ ID NO: 352 - protein, 9 aa, Artificial Sequence
  Synthetic peptide
SLVKHHMYI

SEQ ID NO: 353 - protein, 9 aa, Artificial Sequence
  Synthetic peptide
SLYNTVATL

SEQ ID NO: 354 - protein, 10 aa, Artificial Sequence
  Synthetic peptide
SLYNTVATLY

SEQ ID NO: 355 - protein, 10 aa, Artificial Sequence
  Synthetic peptide
SLYSFPEPEA

SEQ ID NO: 356 - protein, 10 aa, Artificial Sequence
  Synthetic peptide
SMYRVFEVGV

SEQ ID NO: 357 - protein, 9 aa, Artificial Sequence
  Synthetic peptide
SQADALKYV

SEQ ID NO: 358 - protein, 9 aa, Artificial Sequence
  Synthetic peptide
STLCQVEPV

SEQ ID NO: 359 - protein, 9 aa, Artificial Sequence
  Synthetic peptide
STPPPGTRV

SEQ ID NO: 360 - protein, 9 aa, Artificial Sequence
  Synthetic peptide
TLADFDPRV

SEQ ID NO: 361 - protein, 9 aa, Artificial Sequence
  Synthetic peptide
TLDYKPLSV

SEQ ID NO: 362 - protein, 9 aa, Artificial Sequence
  Synthetic peptide
TLFWLLLTL

SEQ ID NO: 363 - protein, 8 aa, Artificial Sequence
  Synthetic peptide
TLFWLLTL

SEQ ID NO: 364 - protein, 9 aa, Artificial Sequence
  Synthetic peptide
TLNAWVKVV

SEQ ID NO: 365 - protein, 9 aa, Artificial Sequence
  Synthetic peptide
TLPGYPPHV

SEQ ID NO: 366 - protein, 10 aa, Artificial Sequence
  Synthetic peptide
TLPPAWQPFL

SEQ ID NO: 367 - protein, 9 aa, Artificial Sequence
  Synthetic peptide
TLQDIVYKL

SEQ ID NO: 368 - protein, 9 aa, Artificial Sequence
  Synthetic peptide
TLSNLSFPV

SEQ ID NO: 369 - protein, 9 aa, Artificial Sequence
  Synthetic peptide
TMNGSKSPV

SEQ ID NO: 370 - protein, 9 aa, Artificial Sequence
  Synthetic peptide
VIFDFLHCI

SEQ ID NO: 371 - protein, 9 aa, Artificial Sequence
  Synthetic peptide
VIMPCSWWV

SEQ ID NO: 372 - protein, 10 aa, Artificial Sequence
  Synthetic peptide
VISNDVCAQV

SEQ ID NO: 373 - protein, 9 aa, Artificial Sequence
  Synthetic peptide
VIVMLTPLV

SEQ ID NO: 374 - protein, 9 aa, Artificial Sequence
  Synthetic peptide
VIYHYVDDL

SEQ ID NO: 375 - protein, 9 aa, Artificial Sequence
  Synthetic peptide
VLAELVKQI

SEQ ID NO: 376 - protein, 9 aa, Artificial Sequence
  Synthetic peptide
VLAGGFFLL

SEQ ID NO: 377 - protein, 9 aa, Artificial Sequence
  Synthetic peptide
VLDFAPPGA

SEQ ID NO: 378 - protein, 9 aa, Artificial Sequence
  Synthetic peptide
VLDGLDVLL

SEQ ID NO: 379 - protein, 9 aa, Artificial Sequence
  Synthetic peptide
VLEETSVML

SEQ ID NO: 380 - protein, 9 aa, Artificial Sequence
  Synthetic peptide
VLFGLGFAI

SEQ ID NO: 381 - protein, 9 aa, Artificial Sequence
  Synthetic peptide
VLHDDLLEA

SEQ ID NO: 382 - protein, 9 aa, Artificial Sequence
  Synthetic peptide
VLLGAVCGV

SEQ ID NO: 383 - protein, 9 aa, Artificial Sequence
  Synthetic peptide
VLMIKALEL

SEQ ID NO: 384 - protein, 9 aa, Artificial Sequence
  Synthetic peptide
VLPLTVAEV

SEQ ID NO: 385 - protein, 9 aa, Artificial Sequence
  Synthetic peptide
VLQELNVTV

SEQ ID NO: 386 - protein, 9 aa, Artificial Sequence
  Synthetic peptide
VLQMKEEDV

SEQ ID NO: 387 - protein, 9 aa, Artificial Sequence
  Synthetic peptide
VLSDFKTWL

SEQ ID NO: 388 - protein, 10 aa, Artificial Sequence
  Synthetic peptide
VLTDGNPPEV

SEQ ID NO: 389 - protein, 10 aa, Artificial Sequence
  Synthetic peptide
VLYRYGSFSV

SEQ ID NO: 390 - protein, 10 aa, Artificial Sequence
  Synthetic peptide
VMNILLQYVV

SEQ ID NO: 391 - protein, 9 aa, Artificial Sequence
  Synthetic peptide
VVTGVLVYL

SEQ ID NO: 392 - skipped

SEQ ID NO: 393 - protein, 9 aa, Artificial Sequence
  Synthetic peptide
WLSLLVPFV

SEQ ID NO: 394 - protein, 10 aa, Artificial Sequence
  Synthetic peptide
YAYDGKDYIA

SEQ ID NO: 395 - protein, 9 aa, Artificial Sequence
  Synthetic peptide
YIGEVLVSV

SEQ ID NO: 396 - protein, 9 aa, Artificial Sequence
  Synthetic peptide
YLEPGPVTA

SEQ ID NO: 397 - protein, 9 aa, Artificial Sequence
  Synthetic peptide
YLEPGPVTV

SEQ ID NO: 398 - protein, 9 aa, Artificial Sequence
  Synthetic peptide
YLFFYRKSV

SEQ ID NO: 399 - protein, 9 aa, Artificial Sequence
  Synthetic peptide
YLGSYGFRL

SEQ ID NO: 400 - protein, 9 aa, Artificial Sequence
  Synthetic peptide
YLIELIDRV

SEQ ID NO: 401 - protein, 9 aa, Artificial Sequence
  Synthetic peptide
YLISGDSPV

SEQ ID NO: 402 - protein, 9 aa, Artificial Sequence
  Synthetic peptide
YLLEMLWRL

SEQ ID NO: 403 - protein, 10 aa, Artificial Sequence
  Synthetic peptide
YLLPRRGPRL

SEQ ID NO: 404 - protein, 10 aa, Artificial Sequence
  Synthetic peptide
YLNDHLEPWI

SEQ ID NO: 405 - protein, 9 aa, Artificial Sequence
  Synthetic peptide
YLNKIQNSL

SEQ ID NO: 406 - protein, 10 aa, Artificial Sequence
  Synthetic peptide
YLNRHLHTWI

SEQ ID NO: 407 - protein, 10 aa, Artificial Sequence
  Synthetic peptide
YLNTVQPTCV

SEQ ID NO: 408 - protein, 10 aa, Artificial Sequence
  Synthetic peptide
YLQLVFGIEV

SEQ ID NO: 409 - protein, 9 aa, Artificial Sequence
  Synthetic peptide
YLQQNWWTL

SEQ ID NO: 410 - protein, 9 aa, Artificial Sequence
  Synthetic peptide
YLQQNWWTL

SEQ ID NO: 411 - protein, 9 aa, Artificial Sequence
  Synthetic peptide
YLQVDLRFL

SEQ ID NO: 412 - protein, 10 aa, Artificial Sequence
  Synthetic peptide
YLQVNSLQTV

SEQ ID NO: 413 - protein, 9 aa, Artificial Sequence
  Synthetic peptide
YLQWIEFSI

SEQ ID NO: 414 - protein, 9 aa, Artificial Sequence
  Synthetic peptide
YLSGADLNL

SEQ ID NO: 415 - protein, 9 aa, Artificial Sequence
  Synthetic peptide
YLSGANLNL

SEQ ID NO: 416 - protein, 9 aa, Artificial Sequence
  Synthetic peptide
YLVGNVCIL

SEQ ID NO: 417 - protein, 9 aa, Artificial Sequence
  Synthetic peptide
YLVSIFLHL

SEQ ID NO: 418 - protein, 10 aa, Artificial Sequence
  Synthetic peptide
YLYQWLGAPV

SEQ ID NO: 419 - protein, 9 aa, Artificial Sequence
  Synthetic peptide
YMCSFLFNL

SEQ ID NO: 420 - protein, 9 aa, Artificial Sequence
  Synthetic peptide
YMDGTMSQV

SEQ ID NO: 421 - protein, 10 aa, Artificial Sequence
  Synthetic peptide
YMLDLQPETT

SEQ ID NO: 422 - protein, 9 aa, Artificial Sequence
  Synthetic peptide
YMNGTMSQV

SEQ ID NO: 423 - protein, 10 aa, Artificial Sequence
  Synthetic peptide
YTCPLCRAPV

SEQ ID NO: 424 - protein, 9 aa, Artificial Sequence
  Synthetic peptide
YTMDGEYRL

SEQ ID NO: 425 - protein, 9 aa, Artificial Sequence
  Synthetic peptide
YVLDHLIVV

SEQ ID NO: 426 - protein, 9 aa, Artificial Sequence
  Synthetic peptide
AIFQSSMTK

SEQ ID NO: 427 - protein, 9 aa, Artificial Sequence
  Synthetic peptide
ALLAVGATK

SEQ ID NO: 428 - protein, 9 aa, Artificial Sequence
  Synthetic peptide
ATGFKQSSK

SEQ ID NO: 429 - protein, 9 aa, Artificial Sequence
  Synthetic peptide
ILRGSVAHK

SEQ ID NO: 430 - protein, 9 aa, Artificial Sequence
  Synthetic peptide
KLCLRFLSK

SEQ ID NO: 431 - protein, 9 aa, Artificial Sequence
  Synthetic peptide
KLGGALQAK

SEQ ID NO: 432 - protein, 9 aa, Artificial Sequence
  Synthetic peptide
KQSSKALQR

SEQ ID NO: 433 - protein, 9 aa, Artificial Sequence
  Synthetic peptide
QVLKKIAQK

SEQ ID NO: 434 - protein, 10 aa, Artificial Sequence
  Synthetic peptide
QVPLRPMTYK

SEQ ID NO: 435 - protein, 9 aa, Artificial Sequence
  Synthetic peptide
RIAAWMATY

SEQ ID NO: 436 - protein, 10 aa, Artificial Sequence
  Synthetic peptide
RISTFKNWPK

SEQ ID NO: 437 - protein, 10 aa, Artificial Sequence
  Synthetic peptide
RLGLQVRKNK

SEQ ID NO: 438 - protein, 9 aa, Artificial Sequence
  Synthetic peptide
RLLFFAPTR

SEQ ID NO: 439 - protein, 9 aa, Artificial Sequence
  Synthetic peptide
RLRAEAQVK

SEQ ID NO: 440 - protein, 9 aa, Artificial Sequence
  Synthetic peptide
RLRPGGKKK

SEQ ID NO: 441 - protein, 9 aa, Artificial Sequence
  Synthetic peptide
RVCEKMALY

SEQ ID NO: 442 - protein, 9 aa, Artificial Sequence
  Synthetic peptide
RVRAYTYSK

SEQ ID NO: 443 - protein, 10 aa, Artificial Sequence
  Synthetic peptide
SVLNYERARR

SEQ ID NO: 444 - protein, 9 aa, Artificial Sequence
  Synthetic peptide
VTLTHPITK

SEQ ID NO: 445 - protein, 10 aa, Artificial Sequence
  Synthetic peptide
YMVPFIPLYR

SEQ ID NO: 446 - protein, 11 aa, Artificial Sequence
  Synthetic peptide
ACQGVGGPGHK

SEQ ID NO: 447 - protein, 9 aa, Artificial Sequence
  Synthetic peptide
ATIGTAMYK

SEQ ID NO: 448 - protein, 9 aa, Artificial Sequence
  Synthetic peptide
AVDLSHFLK

SEQ ID NO: 449 - protein, 10 aa, Artificial Sequence
  Synthetic peptide
AVFDRKSDAK

SEQ ID NO: 450 - protein, 9 aa, Artificial Sequence
  Synthetic peptide
IVTDFSVIK

SEQ ID NO: 451 - protein, 9 aa, Artificial Sequence
  Synthetic peptide
KSMREEYRK

SEQ ID NO: 452 - protein, 9 aa, Artificial Sequence
  Synthetic peptide
NTLEQTVKK

SEQ ID NO: 453 - protein, 10 aa, Artificial Sequence
  Synthetic peptide
RMVLASTTAK

SEQ ID NO: 454 - protein, 9 aa, Artificial Sequence
  Synthetic peptide
SIIPSGPLK

SEQ ID NO: 455 - protein, 10 aa, Artificial Sequence
  Synthetic peptide
SSCSSCPLSK

SEQ ID NO: 456 - protein, 9 aa, Artificial Sequence
  Synthetic peptide
YVNVNMGLK

SEQ ID NO: 457 - protein, 9 aa, Artificial Sequence
  Synthetic peptide
QYDPVAALF

SEQ ID NO: 458 - protein, 9 aa, Artificial Sequence
  Synthetic peptide
AFLPWHRLF

SEQ ID NO: 459 - protein, 9 aa, Artificial Sequence
  Synthetic peptide
AYACNTSTL

SEQ ID NO: 460 - protein, 9 aa, Artificial Sequence
  Synthetic peptide
AYAQKIFKI

SEQ ID NO: 461 - protein, 9 aa, Artificial Sequence
  Synthetic peptide
AYQGVQQKV

SEQ ID NO: 462 - protein, 9 aa, Artificial Sequence
  Synthetic peptide
AYSQQTRGL

SEQ ID NO: 463 - protein, 9 aa, Artificial Sequence
  Synthetic peptide
CYASGWGSI

SEQ ID NO: 464 - protein, 9 aa, Artificial Sequence
  Synthetic peptide
CYTWNQMNL

SEQ ID NO: 465 - protein, 10 aa, Artificial Sequence
  Synthetic peptide
DYCNVLNKEF

SEQ ID NO: 466 - protein, 10 aa, Artificial Sequence
  Synthetic peptide
DYLNEWGSRF

SEQ ID NO: 467 - protein, 9 aa, Artificial Sequence
  Synthetic peptide
DYLQYVLQI

SEQ ID NO: 468 - protein, 9 aa, Artificial Sequence
  Synthetic peptide
EYCPGGNLF

SEQ ID NO: 469 - protein, 9 aa, Artificial Sequence
  Synthetic peptide
EYILSLEEL

SEQ ID NO: 470 - protein, 9 aa, Artificial Sequence
  Synthetic peptide
EYLQLVFGI

SEQ ID NO: 471 - protein, 9 aa, Artificial Sequence
  Synthetic peptide
EYLVSFGVW

SEQ ID NO: 472 - protein, 9 aa, Artificial Sequence
  Synthetic peptide
EYRALQLHL

SEQ ID NO: 473 - protein, 9 aa, Artificial Sequence
  Synthetic peptide
EYYELFVNI

SEQ ID NO: 474 - protein, 9 aa, Artificial Sequence
  Synthetic peptide
GYCTQIGIF

SEQ ID NO: 475 - protein, 9 aa, Artificial Sequence
  Synthetic peptide
IMPKAGLLI

SEQ ID NO: 476 - protein, 9 aa, Artificial Sequence
  Synthetic peptide
IYTWIEDHF

SEQ ID NO: 477 - protein, 10 aa, Artificial Sequence
  Synthetic peptide
KLRGEVKQNL

SEQ ID NO: 478 - protein, 9 aa, Artificial Sequence
  Synthetic peptide
KWLISPVKI

SEQ ID NO: 479 - protein, 9 aa, Artificial Sequence
  Synthetic peptide
KYTSFPWLL

SEQ ID NO: 480 - protein, 10 aa, Artificial Sequence
  Synthetic peptide
KYYLRVRPLL

SEQ ID NO: 481 - protein, 9 aa, Artificial Sequence
  Synthetic peptide
LYQWLGAPV

SEQ ID NO: 482 - protein, 9 aa, Artificial Sequence
  Synthetic peptide
NYQPVWLCL

SEQ ID NO: 483 - protein, 9 aa, Artificial Sequence
  Synthetic peptide
PYLFWLAAI

SEQ ID NO: 484 - protein, 9 aa, Artificial Sequence
  Synthetic peptide
QYDPVAALF

SEQ ID NO: 485 - protein, 10 aa, Artificial Sequence
  Synthetic peptide
RYCNLEGPPI

SEQ ID NO: 486 - protein, 9 aa, Artificial Sequence
  Synthetic peptide
RYLKDQQLL

SEQ ID NO: 487 - protein, 9 aa, Artificial Sequence
  Synthetic peptide
RYLRDQQLL

SEQ ID NO: 488 - protein, 10 aa, Artificial Sequence
  Synthetic peptide
RYNAQCQETI

SEQ ID NO: 489 - protein, 8 aa, Artificial Sequence
  Synthetic peptide
RYPLTFGW

SEQ ID NO: 490 - protein, 10 aa, Artificial Sequence
  Synthetic peptide
RYPLTFGWCF

SEQ ID NO: 491 - protein, 9 aa, Artificial Sequence
  Synthetic peptide
SFHSLHLLF

SEQ ID NO: 492 - protein, 9 aa, Artificial Sequence
  Synthetic peptide
SYRNEIAYL

SEQ ID NO: 493 - protein, 9 aa, Artificial Sequence
  Synthetic peptide
TFPDLESEF

SEQ ID NO: 494 - protein, 9 aa, Artificial Sequence
  Synthetic peptide
TYACFVSNL

SEQ ID NO: 495 - protein, 9 aa, Artificial Sequence
  Synthetic peptide
TYFSLNNKF

SEQ ID NO: 496 - protein, 9 aa, Artificial Sequence
  Synthetic peptide
TYGPVFMCL

SEQ ID NO: 497 - protein, 9 aa, Artificial Sequence
  Synthetic peptide
TYGPVFMSL

SEQ ID NO: 498 - protein, 9 aa, Artificial Sequence
  Synthetic peptide
TYLPTNASL

SEQ ID NO: 499 - protein, 9 aa, Artificial Sequence
  Synthetic peptide
VYALPLKML

SEQ ID NO: 500 - protein, 9 aa, Artificial Sequence
  Synthetic peptide
VYDFAFRDL

SEQ ID NO: 501 - protein, 9 aa, Artificial Sequence
  Synthetic peptide
VYFFLPDHL

SEQ ID NO: 502 - protein, 9 aa, Artificial Sequence
  Synthetic peptide
VYGFVRACL

SEQ ID NO: 503 - protein, 9 aa, Artificial Sequence
  Synthetic peptide
VYGIRLEHF

SEQ ID NO: 504 - protein, 9 aa, Artificial Sequence
  Synthetic peptide
VYLRVRPLL

SEQ ID NO: 505 - protein, 9 aa, Artificial Sequence
  Synthetic peptide
VYYNWQYLL

SEQ ID NO: 506 - protein, 9 aa, Artificial Sequence
  Synthetic peptide
IACPIVMRY

SEQ ID NO: 507 - protein, 10 aa, Artificial Sequence
  Synthetic peptide
KEKYIDQEEL

SEQ ID NO: 508 - protein, 9 aa, Artificial Sequence
  Synthetic peptide
KESTLHLVL

SEQ ID NO: 509 - protein, 9 aa, Artificial Sequence
  Synthetic peptide
LYNTVATLY

SEQ ID NO: 510 - protein, 9 aa, Artificial Sequence
  Synthetic peptide
SFDPIPIHY

SEQ ID NO: 511 - protein, 10 aa, Artificial Sequence
  Synthetic peptide
SFNCRGEFFY

SEQ ID NO: 512 - protein, 9 aa, Artificial Sequence
  Synthetic peptide
IVTDFSVIK

SEQ ID NO: 513 - protein, 10 aa, Artificial Sequence
  Synthetic peptide
TVSGNILTIR

SEQ ID NO: 514 - protein, 9 aa, Artificial Sequence
  Synthetic peptide
APKKPKEPV

SEQ ID NO: 515 - protein, 9 aa, Artificial Sequence
  Synthetic peptide
APRGVRMAV

SEQ ID NO: 516 - protein, 9 aa, Artificial Sequence
  Synthetic peptide
APTKRKGEC

SEQ ID NO: 517 - protein, 9 aa, Artificial Sequence
  Synthetic peptide
DPRRRSRNL

SEQ ID NO: 518 - protein, 9 aa, Artificial Sequence
  Synthetic peptide
GPGHKARVL

SEQ ID NO: 519 - protein, 9 aa, Artificial Sequence
  Synthetic peptide
GPLCKADSL

SEQ ID NO: 520 - protein, 9 aa, Artificial Sequence
  Synthetic peptide
GPRLGVRAT

SEQ ID NO: 521 - protein, 9 aa, Artificial Sequence
  Synthetic peptide
IPRRIRQGL

SEQ ID NO: 522 - protein, 9 aa, Artificial Sequence
  Synthetic peptide
KPTLKEYVL

SEQ ID NO: 523 - protein, 11 aa, Artificial Sequence
  Synthetic peptide
KPYSGTAYNAL

SEQ ID NO: 524 - protein, 11 aa, Artificial Sequence
  Synthetic peptide
KPYSGTAYNSL

SEQ ID NO: 525 - protein, 9 aa, Artificial Sequence
  Synthetic peptide
LPVSCPEDL

SEQ ID NO: 526 - protein, 9 aa, Artificial Sequence
  Synthetic peptide
LPVSPRLQL

SEQ ID NO: 527 - protein, 9 aa, Artificial Sequence
  Synthetic peptide
LPWHRLFLL

SEQ ID NO: 528 - protein, 9 aa, Artificial Sequence
  Synthetic peptide
NPTAQSQVM

SEQ ID NO: 529 - protein, 9 aa, Artificial Sequence
  Synthetic peptide
QPEWFRNVL

SEQ ID NO: 530 - protein, 9 aa, Artificial Sequence
  Synthetic peptide
QPRAPIRPI

SEQ ID NO: 531 - protein, 11 aa, Artificial Sequence
  Synthetic peptide
RPHERNGFTVL

SEQ ID NO: 532 - protein, 9 aa, Artificial Sequence
  Synthetic peptide
RPPIFIRRL

SEQ ID NO: 533 - protein, 13 aa, Artificial Sequence
  Synthetic peptide
RPQGGSRPEFVKL

SEQ ID NO: 534 - protein, 9 aa, Artificial Sequence
  Synthetic peptide
RVRFFFPSL

SEQ ID NO: 535 - protein, 9 aa, Artificial Sequence
  Synthetic peptide
SPERKMLPC

SEQ ID NO: 536 - protein, 9 aa, Artificial Sequence
  Synthetic peptide
SPFFLLLLL

SEQ ID NO: 537 - protein, 9 aa, Artificial Sequence
  Synthetic peptide
SPIVPSFDM

SEQ ID NO: 538 - protein, 11 aa, Artificial Sequence
  Synthetic peptide
SPSVDKARAEL

SEQ ID NO: 539 - protein, 10 aa, Artificial Sequence
  Synthetic peptide
TPGPGVRYPL

SEQ ID NO: 540 - protein, 9 aa, Artificial Sequence
  Synthetic peptide
TPNQRQNVC

SEQ ID NO: 541 - protein, 10 aa, Artificial Sequence
  Synthetic peptide
TPRVTGGGAM

SEQ ID NO: 542 - protein, 9 aa, Artificial Sequence
  Synthetic peptide
VPQYGYLTL

SEQ ID NO: 543 - protein, 9 aa, Artificial Sequence
  Synthetic peptide
AAKGRGAAL

SEQ ID NO: 544 - protein, 8 aa, Artificial Sequence
  Synthetic peptide
APLLRWVL

SEQ ID NO: 545 - protein, 8 aa, Artificial Sequence
  Synthetic peptide
DIYKRWII

SEQ ID NO: 546 - skipped

SEQ ID NO: 547 - protein, 9 aa, Artificial Sequence
  Synthetic peptide
ELKRKMIYM

SEQ ID NO: 548 - protein, 9 aa, Artificial Sequence
  Synthetic peptide
ELNRKMIYM

SEQ ID NO: 549 - protein, 9 aa, Artificial Sequence
  Synthetic peptide
ELRRKMMYM

SEQ ID NO: 550 - protein, 8 aa, Artificial Sequence
  Synthetic peptide
FLKEKGGL

SEQ ID NO: 551 - protein, 9 aa, Artificial Sequence
  Synthetic peptide
FLRGRAYGL

SEQ ID NO: 552 - protein, 9 aa, Artificial Sequence
  Synthetic peptide
GEIYKRWII

SEQ ID NO: 553 - protein, 9 aa, Artificial Sequence
  Synthetic peptide
GFKQSSKAL

SEQ ID NO: 554 - protein, 9 aa, Artificial Sequence
  Synthetic peptide
HSKKKCDEL

SEQ ID NO: 555 - protein, 8 aa, Artificial Sequence
  Synthetic peptide
LPHNHTDL

SEQ ID NO: 556 - protein, 9 aa, Artificial Sequence
  Synthetic peptide
QAKWRLQTL

SEQ ID NO: 557 - protein, 9 aa, Artificial Sequence
  Synthetic peptide
QIKVRVDMV

SEQ ID NO: 558 - protein, 8 aa, Artificial Sequence
  Synthetic peptide
RAKFKQLL

SEQ ID NO: 559 - protein, 8 aa, Artificial Sequence
  Synthetic peptide
YLKDQQLL

SEQ ID NO: 560 - protein, 10 aa, Artificial Sequence
  Synthetic peptide
RLRPGGKKKY

SEQ ID NO: 561 - protein, 9 aa, Artificial Sequence
  Synthetic peptide
ARMILMTHF

SEQ ID NO: 562 - protein, 9 aa, Artificial Sequence
  Synthetic peptide
GRAFVTIGK

SEQ ID NO: 563 - protein, 9 aa, Artificial Sequence
  Synthetic peptide
GRFGLATEK

SEQ ID NO: 564 - protein, 9 aa, Artificial Sequence
  Synthetic peptide
GRFGLATVK

SEQ ID NO: 565 - protein, 10 aa, Artificial Sequence
  Synthetic peptide
KRWIILGLNK

SEQ ID NO: 566 - protein, 11 aa, Artificial Sequence
  Synthetic peptide
KRWIILGLNKI

SEQ ID NO: 567 - protein, 13 aa, Artificial Sequence
  Synthetic peptide
KRWIILGLNKINR

SEQ ID NO: 568 - protein, 13 aa, Artificial Sequence
  Synthetic peptide
KRWIILGLNKIVR

SEQ ID NO: 569 - protein, 13 aa, Artificial Sequence
  Synthetic peptide
KRWIILGLNKIVR

SEQ ID NO: 570 - protein, 8 aa, Artificial Sequence
  Synthetic peptide
KRWIIMGL

SEQ ID NO: 571 - protein, 10 aa, Artificial Sequence
  Synthetic peptide
KRWIIMGLNK

SEQ ID NO: 572 - protein, 9 aa, Artificial Sequence
  Synthetic peptide
RMFPNAPYL

SEQ ID NO: 573 - protein, 10 aa, Artificial Sequence
  Synthetic peptide
RRWIQLGLQK

SEQ ID NO: 574 - protein, 9 aa, Artificial Sequence
  Synthetic peptide
SRYWAIRTR

SEQ ID NO: 575 - protein, 8 aa, Artificial Sequence
  Synthetic peptide
CPNSSIVY

SEQ ID NO: 576 - protein, 10 aa, Artificial Sequence
  Synthetic peptide
EAAGIGILTY

SEQ ID NO: 577 - protein, 9 aa, Artificial Sequence
  Synthetic peptide
EPDLAQCFY

SEQ ID NO: 578 - protein, 11 aa, Artificial Sequence
  Synthetic peptide
EPLPQGQLTAY

SEQ ID NO: 579 - protein, 11 aa, Artificial Sequence
  Synthetic peptide
EPLSQSQITAY

SEQ ID NO: 580 - protein, 9 aa, Artificial Sequence
  Synthetic peptide
HPNIEEVAL

SEQ ID NO: 581 - protein, 11 aa, Artificial Sequence
  Synthetic peptide
HPVAEADYFEY

SEQ ID NO: 582 - protein, 11 aa, Artificial Sequence
  Synthetic peptide
HPVGDADYFEY

SEQ ID NO: 583 - protein, 11 aa, Artificial Sequence
  Synthetic peptide
HPVGEADYFEY

SEQ ID NO: 584 - protein, 11 aa, Artificial Sequence
  Synthetic peptide
HPVGQADYFEY

SEQ ID NO: 585 - protein, 9 aa, Artificial Sequence
  Synthetic peptide
IPSINVHHY

SEQ ID NO: 586 - protein, 9 aa, Artificial Sequence
  Synthetic peptide
IPYLDGTFY

SEQ ID NO: 587 - protein, 13 aa, Artificial Sequence
  Synthetic peptide
LPLNVGLPIIGVM

SEQ ID NO: 588 - protein, 9 aa, Artificial Sequence
  Synthetic peptide
LPSDFFPSV

SEQ ID NO: 589 - protein, 9 aa, Artificial Sequence
  Synthetic peptide
MPFATPMEA

SEQ ID NO: 590 - protein, 9 aa, Artificial Sequence
  Synthetic peptide
NPDIVIYQY

SEQ ID NO: 591 - protein, 10 aa, Artificial Sequence
  Synthetic peptide
VPLDEDFRKY

SEQ ID NO: 592 - protein, 9 aa, Artificial Sequence
  Synthetic peptide
YPLHEQHGM

SEQ ID NO: 593 - protein, 9 aa, Artificial Sequence
  Synthetic peptide
IEDPPFNSL

SEQ ID NO: 594 - protein, 9 aa, Artificial Sequence
  Synthetic peptide
KEKGGLEGL

SEQ ID NO: 595 - protein, 10 aa, Artificial Sequence
  Synthetic peptide
REISVPAEIL

SEQ ID NO: 596 - protein, 8 aa, Artificial Sequence
  Synthetic peptide
IPFYGKAI

SEQ ID NO: 597 - protein, 9 aa, Artificial Sequence
  Synthetic peptide
LPSDFFPSV

SEQ ID NO: 598 - protein, 9 aa, Artificial Sequence
  Synthetic peptide
MPFATPMEA

SEQ ID NO: 599 - protein, 9 aa, Artificial Sequence
  Synthetic peptide
VMAPRTLVL

SEQ ID NO: 600 - protein, 15 aa, Artificial Sequence
  Synthetic peptide
GLNDIFEAQKIEWHE

(19) United States
(12) Patent Application Publication
(10) Pub. No.: US 2021/0380661 A1
(43) Pub. Date: Dec. 9, 2021

(54) Method to Express, Purify, and Biotinylate Eukaryotic Cell-Derived Major Histocompatibility Complexes

(71) Applicant: Texas Tech University System, Lubbock, TX (US)

(72) Inventors: Devin B. Lowe, Abilene, TX (US); Amanda L. Wooster, Abilene, TX (US)

(21) Appl. No.: 17/284,525

(22) PCT Filed: Oct. 15, 2019
(86) PCT No.: PCT/US2019/056281

§ 371 Date: Apr. 12, 2021

Related U.S. Application Data
(60) Provisional application No. 62/746,198, filed on Oct. 16, 2018.

(51) Int. Cl. C07K 14/74

(52) U.S. Cl. CPC C07K 14/70539; C07K 2317/34; C07K 2319/90

(57) ABSTRACT

The present invention includes compositions and methods of making and using a fusion protein comprising a peptide, a first flexible linker, a β2-microglobulin domain, a second flexible linker, a soluble major histocompatibility complex (MHC) heavy chain and a peptide tag.

### CROSS-REFERENCE TO RELATED APPLICATIONS

This application claims priority to U.S. Provisional Application Ser. No. 62/746,198, filed Oct. 16, 2018, the entire contents of which are incorporated herein by reference.

### STATEMENT OF FEDERALLY FUNDED RESEARCH

This invention was made with government support under R15 CA215874 and W81XWH-18-1-0293 awarded by the National Institutes of Health and the Department of Defense, respectively. The government has certain rights in the invention.

### TECHNICAL FIELD OF THE INVENTION

The present invention relates in general to the field of immunology, and more particularly to novel methods for expressing, purifying, and post-translationally modifying eukaryotic cell-derived major histocompatibility complexes.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Oct. 14, 2019, is named TECH2131WO_SeqList.txt and is 114, kilo bytes in size.

### BACKGROUND OF THE INVENTION

Without limiting the scope of the invention, its background is described in connection with methods of making eukaryotic cell-derived major histocompatibility complexes (MHC).

The major histocompatibility complex (MHC) class I and II molecules play an integral role in T cell development and peripheral effector responses (Alcover et al., 2018). MHC class I is retained on the plasma membrane of nucleated cells and consists of a multi-unit heavy chain whose tertiary structure is stabilized by β2 microglobulin through non-covalent forces (Wieczorek et al., 2017). To provide specific binding to antigen specific CD8+ T cells, MHC class I usually retains a short 8-10 amino acid peptide within the MHC peptide binding groove that is derived from degraded intracellular proteins (hereafter referred to as peptide/MHC).

The present understanding of basic T cell properties and dynamics under a variety of normal and diseased settings has been greatly advanced by the ability to produce and purify peptide/MHC for use in assays to specifically engage the T cell receptor (TCR). Arguably the most widespread approach incorporates fluorochrome-conjugated peptide/MHC multimers (e.g., tetramers) for analyzing or isolating antigen-specific CD8+ T cells from biological samples (Khaimar et al., 2018; Soen et al., 2003). Peptide/MHC generation has continued similarly to the process outlined in the landmark work by Altman and colleagues (Altman et al., 1996). Briefly, 02 microglobulin and MHC class I heavy chain (containing a BirA tail) are individually expressed in E. coli and later purified from inclusion bodies through a laborious lysis/solubilization process. A defined MHC class I peptide is then added alongside β2 microglobulin and heavy chain in a precise folding reaction mixture that requires several days to complete prior to affinity chromatography (AC) purification of properly folded peptide/MHC and later biotinylation steps. Although this standard production process works to eventually yield excellent reagents for immunologic assays, there exist a number of major disadvantages. Namely, the standard method is [i] time consuming, [ii] requires substantial levels of raw ingredients (particularly purified MHC class I peptide), and [iii] cannot guarantee large-scale production of properly folded peptide/MHC molecules based on predicted peptide binders. For example, it is extremely difficult to stably produce MHC molecules bearing peptides with low-to-moderate affinity to the MHC peptide-binding groove.

Prior attempts to make peptide/MHC complexes include those of White and colleagues, which designed a soluble HIV-reactive MHC class I molecule (consisting of free heavy chain+linked peptide-β2 microglobulin) for expression in baculovirus, which was capable of biotinylation/multimerization and identifying a particular T cell hybridoma by flow cytometry (White et al., 1999). An additional approach was by Greten and colleagues performed standard plasmid DNA transfection to produce a peptide-β2 microglobulin-heavy chain linked protein in J588L cells that was only capable of dimerization due to mouse IgG fusion (Greten et al., 2002).

Thus a need remains for novel compositions, methods, vectors, cells, etc., that include novel constructs that allow for the production of peptide-MHC complexes in eukaryotic cells.

### SUMMARY OF THE INVENTION

In one embodiment, the present invention includes a fusion protein comprising a peptide, a first flexible linker, a β2-microglobulin domain, a second flexible linker, a soluble major histocompatibility complex (MHC) heavy chain and a peptide tag. In one aspect, the peptide is an immunogenic peptide epitope. In another aspect, the MHC is a human, mouse, rat, hamster, horse, pig, cow, simian, avian, or chimeric MHC. In another aspect, the MHC is Class I MHC. In another aspect, the does not include a transmembrane sequence. In another aspect, the peptide tag is selected from wherein the peptide tag is selected from at least one of: a BirA tail, a myc, a FLAG, a glutathione-S-transferase, a His tag, a maltose binding protein, hemagglutinin (HA)-tag, a V5 tag, a T7 tag, a V9 tag, a NusA-tag, a thioredoxin-tag, or a fluorescent protein-tag, a Her2/neu-tag, a CD20-tag, or a GFP-tag. In another aspect, the first, the second, or both the first and second flexible linkers comprise glycine, serine or both glycine and serine and comprise 5 to 40 residues, but may be 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39. In another aspect, the peptide is 8 to 16 residues long. In another aspect, the peptide is selected from at least one of: ABL1; ACPP; Ad5 ElA; AdV 5 Hexon; AdV Hexon; Ag85A; alfa fetoprotein; ASP-2; BA46; BALF4; BAP31; BCL-2; BCL-2A1; BCL-2L1; BCL-X; BCR-ABL; bcr-abl 210 kD fusion protein; Beta-gal; BGLAP; BMI1; BMLF1; BMRF1; BNP; BRAF 27; BRLF1; bZIP factor; BZLF1; C1orf59; CAMEL; Carbonic anhydrase; CB9L2; CD105; CD33; CD59; CEA; CEACAM; Chondromodulin; circadian clock protein PASD1; CP; CPS; cyclin-dependent kinase 4; CYP190; Cytochrome p450; DEP DC1; DLK1; Dutpase; E6; E7; EBNA 1; EBNA 3A; EBNA 3B; EBNA 6; EBV BRLF1; EBV EBNA3A; EBV LMP2; EDDR1; EGFR; EMNA 3A; EMV-1; Enhanced Green Fluorescent Protein; Env; EphA2; Erkl; ESAT-6; EZH2; FAPa; FLT1; FOLR1; FOXM1; G250; GAD65; Gag; Gag-pol; Glypican 3; gp; gp100; gp33 (C9M); GPC3; H250; HA-1; HA-2; HA-8; HBB; HBsAg; HBV core; HBV polymerase; HBV surface antigen; HCMV IE1; HCMV pp65; HCV core; HCV E; HCV NS3; HCV NS4b; HCV NS5a; HCV NS5B; Heparanase; HER2; HER-2/neu; Histocompatibility antigen 60; HIV gag p24; HIV nef; HIV pol; HIV-1 env gp120; HIV-1 IIIB gp120; HIV-1 nef, HIV-1 p17; HIV-1 RT; HIV-1 US4 gp120; HJURP; HLA-A leader sequence peptide; HLA-A2; HLA-Cw3; Chlamydia trachomatis MOMP; HM1.24; HMMR; HMOX1; HO-1; hPSA; HPV 16 E6; HSP105; HSP90 alpha; hTERT; hTOM34p; hTRT; H-Y; IA-2; IAPP; IDO; IE-1; IE62; IGF; IGRP; IL13r; IL13Ra; Ilr1; Influenza A (PR8) NP; Influenza A MP; Influenza A MP1; Influenza A MP2; Influenza A NP; Influenza A PB1; INFγR; Insulin; Interferon gamma inducible protein (GILT) 30; IRS-2; ITGB8; K8.1; KIF20A; KLK; KLK3; L1; LANA; Large T antigen; Lengsin; Listeria monocytogenes Listeriolysin; LTVIN; LMP-1; LMP-2; LMP-2A; LMP-2A; LY6K; m139; m141; m145; M164; M2-1; M38; M45; M57; MAGE-1; MAGE-10; MAGE-3; MAGE-4; MAGE-A1; MAGEA2; MAGEA3; MAGE-A5; MAGE-C1; MAGE-C2; MART-1; MC38 adpgk neoantigen; MC-38; MCL-1; MCMV IE1; MELK; Mena; Middle T antigen; Midkine; miHAg SMCY; MOG precursor; MP; MPP11; MS4A1; MSLN; Mtb 16 kDa; Mtb 19 kDa; Mtb85A; mTERT; Mucin; muFAPα; MuLV env; Murine Survivin; MYBPC-2; Mycobacterium bovis antigen 85-A; Mycobacterium tuberculosis ESAT-6; Mycobacterium tuberculosis TB10.4; Myelin basic protein; ND; NEF; NEF; Neu/Her-2/Erbb2 proto-oncoprotein; NG2; Non muscle Myosin-9; Non-muscle Myosin; NP; NP396; NP52; NPM1; NRP-1; NRP-2; NRP-V7 superagonist peptide 8.3 Tg NOD mouse; NS3; Nucleocapsid; Nuf2; NY-ESO-1; OVA; Ovalbumin (subdominant); P1A; β2X5a; p53; p56; P79; PAP-3; PASD1; PAX-5; p-Cadherin; PDGFRbeta; PLAC1; PLAM csp; Plasmodium berghei ANKA acid phosphatase; Plasmodium berghei CSP; Plasmodium CSP; Plasmodium falciparum CSP; Plasmodium falciparum Liver stage antigen; PolA; Polymerase; Polyprotein; pp65; PPE; Pr1; PRAME; Prominin1; PSA; PSCA; PSM β2; PSMA; RGS5; RhoC; RNF43; RSV A strain F protein; RSV NP; RT; S protein; SAA; SART3; Sialidases; SIV gag; SMCY; Spike GP; STEAPI; Surface IgG (sA20-Ig) of A20; Survivin; Survivin-3a; SV40 T antigen; TACE; TARP; TARP 2M; Tax; TB10.3-4; Telomerase; TEM1; tgd057; TGFβ; TNPO3; topII; TPBG; TPR-protein; TRAG; TRP2; Trypanosoma cruzi ASP-2; Trypanosoma cruzi SP; TTK; Tyrosinase; Tyrosine-3-hydroxylase; Ubiquitin; UL105; UL138; UL44; USP9Y; V131; Vaccinia virus Copenhagen Protein G5; Vaccinia virus Host range protein 2; VACCL3_100; VEGFR1; VEGFR2; VEGFR2/KDR fragment 1; Vif; Vinculin; VP1; Vpu; VSV N; West Nile virus NY-99 polyprotein precursor; West Nile Virus polyprotein; WT1; Yellow Fever Virus 17D polyprotein; or ZnT-8. In another aspect, the MHC is selected from at least one of H-2 Db, H-2 Dd; H-2 Dk; H-2 Kb; H-2 Kd; H-2 Kk; H-2 Ld; HLA-A*0101; HLA-A*0201; HLA-A*0301; HLA-A*1101; HLA-A*2301; HLA-A*2402; HLA-A*2902; HLA-A*6801; HLA-B*0702; HLA-B*0801; HLA-B*1501; HLA-B*2705; HLA-B*3501; HLA-B*4001; HLA-B*5101; or HLA-E*0101. In another aspect, the peptide is at least one of SEQ ID NO:1-599.

In another embodiment, the present invention includes a nucleic acid that expresses a fusion protein comprising a peptide, a first flexible linker, a β2-microglobulin domain, a second flexible linker, a soluble major histocompatibility complex (MHC) heavy chain and a peptide tag. In one aspect, the peptide is an immunogenic peptide epitope. In another aspect, the MHC is a human, mouse, rat, hamster, horse, pig, cow, simian, avian, or chimeric MHC. In another aspect, the MHC is Class I MHC. In another aspect, the MHC does not include a transmembrane sequence. In another aspect, the peptide tag is selected from at least one of: a BirA tail, a myc, a FLAG, a glutathione-S-transferase, a His tag, a maltose binding protein, hemagglutinin (HA)-tag, a V5 tag, a T7 tag, a V9 tag, a NusA-tag, a thioredoxin-tag, or a fluorescent protein-tag, a Her2/neu-tag, a CD20-tag, or a GFP-tag. In another aspect, the first, the second, or both the first and second flexible linkers comprise glycine, serine or both glycine and serine and comprise 5 to 40 residues. In another aspect, the peptide is 8 to 16 residues long. In another aspect, the peptide is selected from at least one of: ABL1; ACPP; Ad5 ElA; AdV 5 Hexon; AdV Hexon; Ag85A; alfa fetoprotein; ASP-2; BA46; BALF4; BAP31; BCL-2; BCL-2A1; BCL-2L1; BCL-X; BCR-ABL; bcr-abl 210 kD fusion protein; Beta-gal; BGLAP; BMI1; BMLF1; BMRF1; BNP; BRAF 27; BRLF1; bZIP factor; BZLF1; C1orf59; CAMEL; Carbonic anhydrase; CB9L2; CD105; CD33; CD59; CEA; CEACAM; Chondromodulin; circadian clock protein PASD1; CP; CPS; cyclin-dependent kinase 4; CYP190; Cytochrome p450; DEP DC1; DLK1; Dutpase; E6; E7; EBNA 1; EBNA 3A; EBNA 3B; EBNA 6; EBV BRLF1; EBV EBNA3A; EBV LMP2; EDDR1; EGFR; EMNA 3A; EMV-1; Enhanced Green Fluorescent Protein; Env; EphA2; Erkl; ESAT-6; EZH2; FAPa; FLT1; FOLR1; FOXM1; G250; GAD65; Gag; Gag-pol; Glypican 3; gp; gp100; gp33 (C9M); GPC3; H250; HA-1; HA-2; HA-8; HBB; HBsAg; HBV core; HBV polymerase; HBV surface antigen; HCMV IE1; HCMV pp65; HCV core; HCV E; HCV NS3; HCV NS4b; HCV NS5a; HCV NS5B; Heparanase; HER2; HER-2/neu; Histocompatibility antigen 60; HIV gag p24; HIV nef; HIV pol; HIV-1 env gp120; HIV-1 IIIB gp120; HIV-1 nef; HIV-1 p17; HIV-1 RT; HIV-1 US4 gp120; HJURP; HLA-A leader sequence peptide; HLA-A2; HLA-Cw3; Chlamydia trachomatis MOMP; HM1.24; HMMR; HMOX1; HO-1; hPSA; HPV 16 E6; HSP105; HSP90 alpha; hTERT; hTOM34p; hTRT; H-Y; IA-2; IAPP; IDO; IE-1; IE62; IGF; IGRP; IL13r; IL13Ra; Ilr1; Influenza A (PR8) NP; Influenza A MP; Influenza A MP1; Influenza A MP2; Influenza A NP; Influenza A PB1; INFyR; Insulin; Interferon gamma inducible protein (GILT) 30; IRS-2; ITGB8; K8.1; KIF20A; KLK; KLK3; L1; LANA; Large T antigen; Lengsin; Listeria monocytogenes Listeriolysin; LIVIN; LMP-1; LMP-2; LMP-2A; LMP-2A; LY6K; m139; m141; m145; M164; M2-1; M38; M45; M57; MAGE-1; MAGE-10; MAGE-3; MAGE-4; MAGE-A1; MAGEA2; MAGEA3; MAGE-A5; MAGE-C1; MAGE-C2; MART-1; MC38 adpgk neoantigen; MC-38; MCL-1; MCMV IE1; MELK; Mena; Middle T antigen; Midkine; miHAg SMCY; MOG precursor; MP; MPP11; MS4A1; MSLN; Mtb 16 kDa; Mtb 19 kDa: Mtb85A; mTERT; Mucin; muFAPα; MuLV env; Murine Survivin; MYBPC-2; Mycobacterium bovis antigen 85-A; Mycobacterium tuberculosis ESAT-6; Mycobacterium tuberculosis TB10.4; Myelin basic protein; ND; NEF; NEF; Neu/Her-2/Erbb2 proto-oncoprotein; NG2; Non muscle Myosin-9; Non-muscle Myosin; NP; NP396; NP52; NPM1; NRP-1; NRP-2; NRP-V7 superagonist peptide 8.3 Tg NOD mouse; NS3; Nucleocapsid; Nuf2; NY-ESO-1; OVA; Ovalbumin (subdominant); P1A; β2X5a; p53; p5⁶; P79; PAP-3; PASD1; PAX-5; p-Cadherin; PDGFRbeta; PLAC1; PLAM csp; Plasmodium berghei ANKA acid phosphatase; Plasmodium berghei CSP; Plasmodium CSP; Plasmodium falciparum CSP; Plasmodium falciparum Liver stage antigen; PolA; Polymerase; Polyprotein; pp65; PPE; Pr1; PRAME; Prominin1; PSA; PSCA; PSM β2; PSMA; RGS5; RhoC; RNF43; RSV A strain F protein; RSV NP; RT; S protein; SAA; SART3; Sialidases; SIV gag; SMCY; Spike GP; STEAPI; Surface IgG (sA20-Ig) of A20; Survivin; Survivin-3a; SV40 T antigen; TACE; TARP; TARP 2M; Tax; TB10.3-4; Telomerase; TEM1; tgd057; TGFβ; TNP03; topII; TPBG; TPR-protein; TRAG; TRP2; Trypanosoma cruzi ASP-2; Trypanosoma cruzi SP; TTK; Tyrosinase; Tyrosine-3-hydroxylase; Ubiquitin; UL105; UL138; UL44; USP9Y; V131; Vaccinia virus Copenhagen Protein G5; Vaccinia virus Host range protein 2; VACCL3_100; VEGFR1; VEGFR2; VEGFR2/KDR fragment 1; Vif, Vinculin; VP1; Vpu; VSV N; West Nile virus NY-99 polyprotein precursor; West Nile Virus polyprotein; WT1; Yellow Fever Virus 17D polyprotein; or ZnT-8. In another aspect, the MHC is selected from at least one of H-2 Db, H-2 Dd; H-2 Dk; H-2 Kb; H-2 Kd; H-2 Kk; H-2 Ld; HLA-A*0101; HLA-A*0201; HLA-A*0301; HLA-A*1101; HLA-A*2301; HLA-A*2402; HLA-A*2902; HLA-A*6801; HLA-B*0702; HLA-B*0801; HLA-B*1501; HLA-B*2705; HLA-B*3501; HLA-B*4001; 1HLA-B*5101; or HLA-E*0101. In another aspect, the peptide is at least one of SEQ ID NO:1-599.

In another embodiment, the present invention includes a method of making a soluble eukaryotic-derived peptide/MHC complex comprising: expressing in a cell a fusion protein comprising a peptide, a first flexible linker, a β2-microglobulin domain, a second flexible linker, a soluble major histocompatibility complex (MHC) heavy chain and a peptide tag. In another aspect, the method further comprises isolating the fusion protein from a supernatant. In another aspect, the method further comprises forming dimers, trimers, tetramers, or multimers of the fusion protein by mixing the fusion protein with one or more agents that bind to two or more fusion proteins. In another aspect, the agent is selected from an antibody, a cross-linking agent, a ligase, an avidin, a streptavidin, a Protein A, or a J-chain. In another aspect, the peptide is an immunogenic peptide epitope. In another aspect, the MHC is a human, mouse, rat, hamster, horse, pig, cow, simian, avian or chimeric MHC. In another aspect, the MHC is Class I MHC. In another aspect, the MHC does not include a transmembrane sequence. In another aspect, the peptide tag is selected from at least one of a BirA tail, a myc, a FLAG, a glutathione-S-transferase, a His tag, a maltose binding protein, hemagglutinin (HA)-tag, a V5 tag, a T7 tag, a V9 tag, a NusA-tag, a thioredoxin-tag, or a fluorescent protein-tag, a Her2/neu-tag, a CD20-tag, or a GFP-tag. In another aspect, the irst, the second, or both the first and second flexible linkers comprise glycine, serine or both glycine and serine and comprise 5 to 40 residues. In another aspect, the peptide is 8 to 16 residues long.

In another embodiment, the present invention includes a cell line expressing a fusion protein comprising a fusion protein comprising a peptide, a first flexible linker, a β2-microglobulin domain, a second flexible linker, a soluble major histocompatibility complex (MHC) heavy chain and a peptide tag. In one aspect, the fusion protein is integrated into the genome by co-transfecting a fusion protein expressing vector with a transposase vector that expresses a transposase and wherein the fusion protein expressing vector, the transposase vector, or both further comprise a selectable marker. In one aspect, the peptide is an immunogenic peptide epitope. In another aspect, the MHC is a human, mouse, rat, hamster, horse, pig, cow, simian, avian, or chimeric MHC. In another aspect, the MHC is Class I MHC. In another aspect, the MHC does not include a transmembrane sequence. In another aspect, the peptide tag is selected from at least one of: a BirA tail, a myc, a FLAG, a glutathione-S-transferase, a His tag, a maltose binding protein, hemagglutinin (HA)-tag, a V5 tag, a T7 tag, a V9 tag, a NusA-tag, a thioredoxin-tag, or a fluorescent protein-tag, a Her2/neu-tag, a CD20-tag, or a GFP-tag. In another aspect, the first, the second, or both the first and second flexible linkers comprise glycine, serine or both glycine and serine and comprise 5 to 40 residues. In another aspect, the peptide is 8 to 16 residues long.

### BRIEF DESCRIPTION OF THE DRAWINGS

For a more complete understanding of the features and advantages of the present invention, reference is now made to the detailed description of the invention along with the accompanying figures and in which:

FIG. 1A shows a representative DNA schematic of the synthetic peptide/MHC complex. The designed peptide/MHC molecule contains distinct peptide, beta-2-microglobulin, and MHC heavy chain regions that are linked via explicit glycine/serine amino acids. The MHC heavy chain lacks a transmembrane domain (designated sHeavy chain), ensuring that properly folded peptide/MHC protein is secreted from cells into the culture medium. The peptide/MHC construct also contains a terminal BirA tail for enzymatic conjugation of biotin. FIG. 1B shows a standard workflow for expressing, purifying, and biotinylating eukaryotic-derived peptide/MHC molecules. [1] A suitable eukaryotic host cell line (such as CHO cells) is transiently transfected with two vectors that allow for transposon-directed integration of genes of interest. Vector 1 encodes the SB transposase (SB100X) while vector 2 is a transposon-compatible plasmid that encodes the synthetic peptide/MHC complex and puromycin resistance transgenes. [2] A stable cell line secreting peptide/MHC complexes is generated in as little as 2 weeks following antibiotic selection and expansion. [3] Spent culture media is processed through AC (against the sHeavy chain) to selectively purify peptide/MHC complexes. [4] A biotin ligase may then be employed to enzymatically conjugate biotin to the BirA tail of the synthetic peptide/MHC protein. [5] Following an additional polishing step such as size exclusion chromatography and [6] multimerization steps, the peptide/MHC reagent can be incorporated into immunologically-relevant assays that, for example, detect antigen-specific T cells. Abbreviation used: secretory MHC class I heavy chain (sHeavy chain), major histocompatibility complex (MHC)

FIGS. 2A and 2B show that stable CHO cells lines were established by a SB transposon system to secrete peptide/MHC molecules into culture media. (FIG. 2A) Evidence of extracellular peptide/MHC protein was first determined from cell-free supernatants by SDS-PAGE and coomassie blue staining. (FIG. 2B) Representative chromatogram of small scale AC purification of peptide/MHC-containing media. Cell-free supernatant was passed through an equilibrated agarose bead column containing the MHC class I-reactive antibody M1/42. After washing away unbound material, peptide/MHC protein was eluted, buffer-exchanged, and concentrated. Protein purity was then assessed using SDS-PAGE and coomassie blue staining. Arrow inset indicating peptide/MHC around the predicted molecular weight. Abbreviation used: protein ladder (L), affinity chromatography (AC), preparation (prep)

FIG. 3A shows Purified peptide/MHC (i.e., SIINFEKL/H-2 Kb) identify was confirmed by western blot using monoclonal antibodies reactive to the β2 microglobulin and BirA tail regions of the design molecule as depicted in FIG. 1A. FIG. 3B shows that peptide/MHC ligand binding was determined through immunoprecipitation and western blot following incubation of SIINFEKL/H-2 Kb and a TCR-like antibody specific to this particular peptide/MHC class I complex. AC-purified peptide/MHC was also incorporated as a positive control. Abbreviations used: protein ladder (L), SIINFEKL epitope+MHC class I (SIINFEKL/H-2 Kb), isotype (Iso), positive (Pos), control (ctrl)

FIGS. 4A to 4C show that the peptide/MHC was biotinylated as detailed in the Materials and Methods and specific streptavidin binding initially confirmed through (FIG. 4A) western blot and (FIG. 4B) ELISA using wells coated with streptavidin. (FIG. 4C) Biotinylated peptide/MHC was also incubated with streptavidin-conjugated 5 μm beads and washed extensively. Beads were then exposed to isotype, anti-SIINFEKL/H-2 Kb, or anti-MHC monoclonal antibodies followed by washing steps and incubation with relevant secondary PE-conjugated antibodies. Specific ligand reactivity was subsequently determined by flow cytometry. Abbreviations used: protein ladder (L), SIINFEKL epitope+MHC class I (SIINFEKL/H-2 Kb), biotinylated SIINFEKL/H-2 Kb (b-SIINFEKL/H-2 Kb), positive (Pos), isotype (Iso), control (ctrl), major histocompatibility complex (MHC), horseradish peroxidase (HRP), streptavidin (SA), primary antibody (1° Ab)

FIG. 5 shows that the biotinylated peptide/MHC was incubated with PE-conjugated streptavidin to produce “small-scale” batches of SIINFEKL-reactive tetramers. Purified CD8+ T cells harvested from either wild-type or OT-1 mice were incubated with tetramers, washed, and stained with an anti-CD8 FITC antibody. Cells were again washed, fixed, and analyzed by flow cytometry for ligand binding. Abbreviations used: wild-type (WT), streptavidin (SA)

FIG. 6 shows the determination of the ability to construct and express a membrane-bound OVA-specific peptide/MHC molecule (i.e., SIINFEKL/H-2 Kb). 4T1 cells were transiently transfected with two plasmids that allow transposon-directed genomic integration and cultured over a period of two weeks in puromycin-containing culture media (as outlined in FIG. 1B). The remaining “stably engineered” cells were then incubated with either an isotype control antibody or antibody that binds the OVA-specific epitope SIINFEKL within the constraints of MHC class I and assessed for reactivity by flow cytometry. Abbreviations used: SIINFEKL epitope+MHC class I (SIINFEKL/H-1-2 Kb), major histocompatibility complex (MHC)

FIG. 7 is a DNA coding sequence of the synthetic SIINFEKL/H-2 Kb molecule (SEQ ID NO: 600).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present invention includes compositions, vectors, cells, and methods of making MHC class I-specific reagents such as fluorescently-labeled multimers (e.g., tetramers) that can be used to study CD8+ T cells under normal and diseased states. However, recombinant MHC class I components (comprising MHC class I heavy chain and p2 microglobulin) are usually produced in bacteria following a lengthy purification protocol that requires additional non-covalent folding steps with exogenous peptide for complete molecular assembly. The present inventors have developed an alternative and rapid approach to generating soluble and fully-folded MHC class I molecules in eukaryotic cell lines (such as CHO cells) using, e.g., a Sleeping Beauty transposon system. Importantly, this method generates stable cell lines that reliably secrete epitope-defined MHC class I molecules into the tissue media for convenient purification and eventual biotinylation/multimerization. Additionally, MHC class I components are covalently linked, providing the opportunity to produce a diverse set of CD8+ T cell-specific reagents bearing peptides with various affinities to MHC class I.

As used herein, the term “gene” refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “vector” is used in reference to nucleic acid molecules that transfer DNA segment(s) from one cell to another. The vector may be further defined as one designed to propagate specific sequences, or as an expression vector that includes a promoter operatively linked to the specific sequence, or one designed to cause such a promoter to be introduced. The vector may exist in a state independent of the host cell chromosome, or may be integrated into the host cell chromosome

As used herein, the term “host cell” refers to cells that have been engineered to contain nucleic acid segments or altered segments, whether archeal, prokaryotic, or eukaryotic. Thus, engineered, or recombinant cells, are distinguishable from naturally occurring cells that do not contain recombinantly introduced genes through the hand of man.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it effects the transcription of the sequence; or a ribosome binding site is operably linked to e coding sequence if it is positioned so as to facilitate translation. Generally, “operably linked” refers to a DNA sequences being linked are contiguous and, in the case of a secretory leader, contiguous and in same reading frame. Enhancers do not have to be contiguous. Linking is accomplished by ligation at convenient restriction sites. If such sites do not exist, then synthetic oligonucleotide adaptors or linkers are used in accord with conventional practice.

As used herein, the terms “cell” and “cell culture” are used interchangeably end all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Different designations are will be clear from the contextually clear.

As used herein, the term “plasmid” are designated by a lower case p preceded and/or followed by capital letters and/or numbers and refer to self-replicating circular DNA that include an origin of replication, and typically one or more selectable markers. The starting plasmids herein are commercially available, are publicly available on an unrestricted basis, or can be constructed from such available plasmids in accord with published procedures. In addition, other equivalent plasmids are known in the art and will be apparent to the ordinary artisan.

As used herein, the term “selectable marker” refers to the use of a gene that encodes an enzymatic activity and which confers the ability to grow in medium lacking what would otherwise be an essential nutrient (e.g., the HIS3 gene in yeast cells); in addition, a selectable marker may confer resistance to an antibiotic or drug upon the cell in which the selectable marker is expressed. A review of the use of selectable markers in mammalian cell lines is provided in Sambrook, J., et al., MOLECULAR CLONING: A LABORATORY MANUAL, 2nd ed., Cold Spring Harbor Laboratory Press, New York (1989) pp. 160.9-160.15.

As used herein the terms “protein”, “polypeptide” or “peptide” refer to compounds comprising amino acids joined via peptide bonds and are used interchangeably.

As used herein, the terms “fusion protein” or “chimeric protein” refer to a hybrid protein, that includes portions of two or more different polypeptides, or fragments thereof, resulting from the expression of a polynucleotide that encodes at least a portion of each of the two polypeptides.

As used herein, the term “transformation,” refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome.

As used herein, the term “transfection” refers to the introduction of foreign DNA into eukaryotic cells. Transfection may be accomplished by a variety of methods known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell that has stably integrated foreign DNA into the genomic DNA. The term also encompasses cells that transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. As used herein, the term “transient transfectant” refers to cells that have taken up foreign DNA but have failed to integrate this DNA.

The present invention can be used to circumvent drawbacks in the prior art, in particular, stabilizing peptide binding to the MHC peptide binding groove. Previous efforts have revealed the ability to engineer and produce peptide/MHC molecules in bacteria by covalently joining the MHC class I peptide, β2 microglobulin, and heavy chain with discrete amino acid linkers (designated single-chain trimers [SCTs]) (Yu et al., 2002). For most SCTs reported, these engineered proteins fold correctly and specifically engage CD8+ T cells as tetramers (Mitaksov et al., 2007), irrespective of the artificial linker design (Hansen et al., 2009). However, this particular SCT method still utilizes a bacterial expression system and requires substantial purification and refolding efforts.

The present inventors have developed an alternative method to potentially improve the production of peptide/MHC based on the SCT approach. The present invention has the ability to rapidly generate eukaryotic cell lines that stably express and secrete peptide/MHC into the tissue media for purification and biotinylation. This novel protocol provides a much faster/convenient route to generating properly folded peptide/MHC with minimal user intervention, especially for MHC class I targets with high demand (such as the model OVA epitope SIINFEKL). By using a SCT strategy, it was possible to generate MHC molecules presenting a range of class I peptides (i.e., low-to-high binding affinity), which can be reliably generated. Additionally, these eukaryotic-derived peptide/MHC molecules can be used to recapitulate binding dynamics with TCRs in downstream assays (Schmidt and Lill 2018).

Mice. Female 6-8-week-old C57BL/6J (stock #000664) and OT-1 (stock #003831) mice were purchased from The Jackson Laboratory (Bar Harbor, Me., USA) and maintained in micro-isolator cages under sterile conditions. Animals were humanely euthanized and spleens/lymph nodes harvested and combined for Ficoll gradient centrifugation (GE HealthCare, Piscataway, N.J.). The lymphocyte interphase was then subjected to ACK lysis and eventual CD8+ T cell purification using MACS bead positive selection as instructed by the manufacturer (Miltenyi Biotec, Cambridge, Mass.). Purified CD8+ T cells were aliquoted in 90% FBS/10% DMSO and stored in liquid nitrogen until use. All mouse procedures were followed in accordance with TTUHSC IACUC-approved protocols.

Cell lines and culture. FreeStyle™ Chinese Hamster Ovary (CHO-S) (Thermo Fisher Scientific, Waltham, Mass.) and 4T1 (ATCC, Manassas, Va.) cells were utilized for in vitro studies. CHO-S cells were passaged in FreeStyle™ CHO Expression Medium (Thermo Fisher Scientific) according to the manufacturer's recommendations. 4T1 cells are naturally deficient in H-2 Kb expression and were grown in RPMI 1640 supplemented with 10% heat-inactivated FBS, 100 U/ml penicillin, 100 μg/ml streptomycin, and 10 mmol/l L-glutamine (all from Thermo Fisher Scientific). All cell lines were maintained in vented flasks at 37° C. with 5% CO₂.

Cloning strategy and construction of transposon expression vectors. Full-length mouse p2 microglobulin (NCBI Reference Sequence: NM 009735.3) and MHC class I heavy chain (H-2 Kb) (NCBI Reference Sequence: NM_001001892.2), relevant sequences incorporated herein by reference, cDNA were synthesized from C57BL/6J splenocytes following TRIzol lysis (Thermo Fisher Scientific) and RT-PCR using oligo(dT) primers (SuperScript IV First-Strand Synthesis Kit; Thermo Fisher Scientific). Secretory MHC class I heavy chain was designed to not include the transmembrane domain. The leader signal, SIINFEKL epitope, and Gly/Ser amino acid linkers were ultimately added by overhang PCR as previously reported (Hansen et al., 2009) using the Phusion High-Fidelity DNA Polymerase (Thermo Scientific Fisher). PCR fragments were gel excised/purified and ligated (LigaFast™ Rapid DNA Ligation System; Promega, Madison, Wis.) into puc19 (NEB, Ipswich, Mass.) via SacI/HindIII restriction enzyme sites and fragments pieced together using the unique NheI/BamHI sites of the synthetic peptide/MHC sequence. The BirA AviTag™ amino acid sequence (GLNDIFEAQKIEWHE SEQ ID NO: 600) was subsequently added to the construct's terminus by overlap-extension PCR and cloned into a separate puc19 holding vector. Full-length peptide/MHC was then amplified and cloned into the pSBbi-pur transposon vector (kindly provided by Dr. Eric Kowarz [Addgene plasmid #60523]) using the SfI restriction enzyme sites (Kowarz et al., 2015). Plasmid transformations were carried out in chemically-competent NEB-5 alpha E. coli (NEB) using ampicillin selection. All vector constructs were confirmed by restriction enzyme digestion and DNA sequencing.

Sleeping Beauty (SB) transposon system. Parental cell lines were transiently transfected with transposon-related vectors using Lipofectamine reagent (Thermo Fisher Scientific) as directed by the manufacturer. A plasmid encoding the SB 100× transposase (pCMV[CAT]T7-SB100; designated Vector 1), a gift from Dr. Zsuzsanna Izsvak (Addgene plasmid #34879), and a transposon plasmid containing the necessary inverted terminal repeats (pSBbi-pur; designated Vector 2) were used. Both vectors were provided concurrently to stably integrate peptide/MHC transgenes into cells. Briefly, 1×10⁵ cells were plated in 24-well plates (Corning, Corning N.Y.) and exposed to Vector 1 (12.5 ng)/Vector 2 (488 ng) in a total volume of 500 μl media as similarly described (Kowarz et al., 2015). Culture media was replenished with 2 ml fresh media after 24 hrs and 48 hrs, whereupon cells were exposed to lethal doses of puromycin (CHO-S—10 μg/ml; 4T1—5 μg/ml) (Invivogen, San Diego, Calif.). Fresh media containing puromycin was provided every 2-3 days as needed. Generally, actively dividing cells were ready for expansion by 2 weeks, with virtually all cell clones expressing peptide/MHC molecules.

SDS-PAGE and western blot. To remove extraneous tissue media components such as surfactants, CHO cell supernatants were passed onto PD10 columns (GE Healthcare) and concentrated/washed in PBS using a 30 MWCO Amicon centrifugal unit (MilliporeSigma, Burlington, Mass.). Protein concentration was determined using a BCA protein assay kit (Thermo Scientific Fisher) and sample aliquots stored at −80° C. until use. Protein purity was assessed with SDS-PAGE and coomassie blue staining while protein identity was confirmed through western blotting. Briefly, proteins were resolved on a 4%/12% polyacrylamide gel, transferred to a PVDF membrane (Amersham Hybond, 0.2 μm; GE Healthcare), and blocked with 5% milk in PBST (0.1% Tween 20 in PBS) for 1 hr at RT. Membranes were then incubated with various primary antibodies (1 μg/ml) in block solution with rocking at 4° C. overnight. Specific primary reagents included anti-mouse β₂ microglobulin (clone 893803; R&D Systems, Minneapolis, Minn.) or anti-BirA tail (clone Abc; Avidity, Aurora, Colo.) antibodies. Blots were then washed extensively with PBST and incubated in block with secondary HRP-conjugated goat antibodies specific to rat IgG (H+L) or mouse IgG (H+L) (Thermo Scientific Fisher) for 1 hr at RT. Washed blots were finally developed with a SignalFire ECL reagent (Cell Signaling, Danvers, Mass.) and exposed/imaged on a ChemiDoc™ Touch Imaging System (Bio-Rad, Hercules, Calif.). In separate experiments, blots containing biotinylated protein were probed with a Streptavidin-HRP reagent (Thermo Scientific Fisher) for 1 hr at RT to confirm streptavidin binding potential.

Immunoprecipitation. A Pierce Classic IP kit (Thermo Scientific Fisher) was used to investigate ligand binding of soluble peptide/MHC protein. Peptide/MHC protein was incubated overnight at 4° C. with 2 μg of the anti-SIINFEKL (clone eBio25-D1.16; Thermo Scientific Fisher) or mouse IgG isotype (clone MOPC-21; MP Biomedicals, Santa Ana, Calif.) antibodies. The anti-SIINFEKL antibody functions as a TCR-like antibody (Lowe et al., 2017), and binds SIINFEKL/H-2 Kb much like SIINFEKL-reactive T cells such as OT-1 CD8+ T cells. Following purification of IgG containing complexes, samples were boiled and analyzed by western blot as described above.

Affinity chromatography (AC). The MHC class I-reactive M1/42 antibody (Bio X Cell, West Lebanon, N.H.) was covalently bound to an NHS-activated agarose bead column (HiTrap™ NHS-activated HP; GE Healthcare) manually as directed by the manufacturer. The column was attached to an ÄKTA™ start chromatography system (GE Healthcare) for automatic operation and equilibrated with 20 mM sodium phosphate, pH 7.0 (binding buffer). Cell-free supernatants were first desalted in PBS using PD-10 columns and diluted 1:2 in binding buffer prior to AC. Samples were then applied to the M1/42 column, unbound material washed away with binding buffer, and peptide/MHC molecules eluted using 0.1 M glycine, pH 2.7. Eluates were dispensed in tubes containing 1 M Tris, pH 9 and relevant fractions combined and concentrated/washed in PBS as explained above.

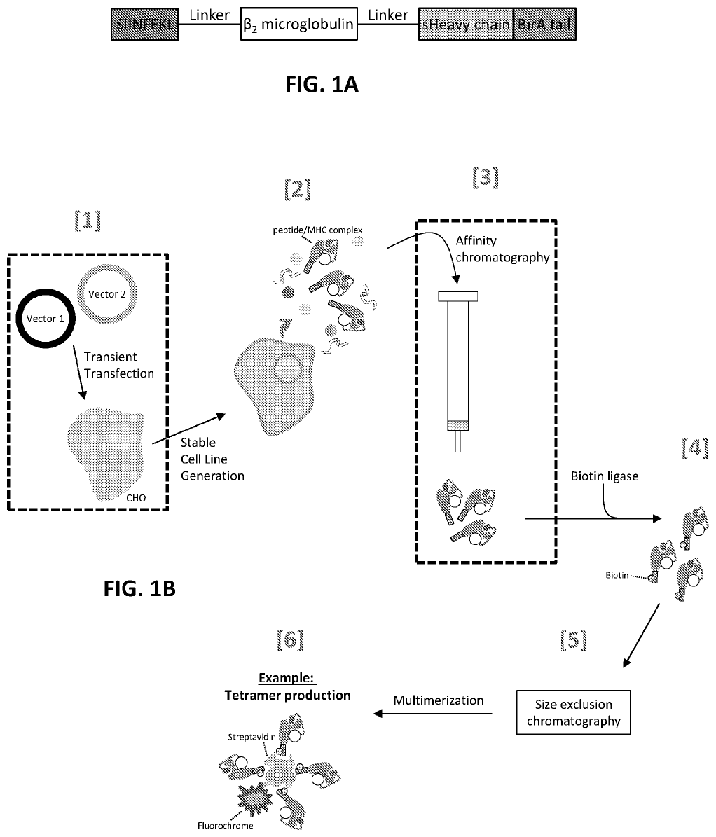

Biotinylation and tetramerization. Peptide/MHC was biotinylated using 2.5 μg BirA ligase (Avidity) overnight at 4° C. according to the manufacturer's guidelines. Reaction components were removed by successive washes in PBS using a 30 MWCO centrifugal filter (MilliporeSigma). Alternatively, larger reaction mixtures could be exclusively polished through size exclusion chromatography (Altman and Davis 2016) using a HiPrep™ 16/60 Sephacryl™ S-200 HR column (GE Healthcare) as indicated in FIG. 1B. In order to produce small-scale tetramer batches, biotinylated peptide/MHC was incubated with PE-conjugated streptavidin (BD Biosciences, San Jose, Calif.) at a 4:1 molar ratio in the dark for 30 min at RT. Tetramers were then stored at 4° C. shielded from light.

Enzyme-Linked Immunosorbent Assay (ELISA). To investigate the success of peptide/MHC biotinylation, a 96-well high protein binding plate (Corning) was first coated overnight at 4° C. with 4 μg/ml streptavidin (Promega) in 0.1 M sodium carbonate and then blocked (3% BSA/PBS) for 1 hr at RT. Biotinylated protein samples were diluted in block and added to wells at various dilutions and incubated at RT for 1 hr. Wells were washed extensively with PBST and then exposed to 4 μg/ml of either a MHC class I-reactive (clone M1/42; Biolegend) or rat isotype control IgG (Biolegend) antibody in block. In separate wells, a positive control biotinylated irrelevant rat antibody (Biolegend) was incorporated to validate the extent of streptavidin binding. The plate was again washed with PBST and relevant wells provided a goat anti-rat HRP (Thermo Scientific Fisher) or goat anti-mouse FcγR-specific HRP (Jackson ImmunoResearch, West Grove, Pa.) antibody in block for 1 hr at RT. Wells were washed with PBST and developed for 5 min after the addition of 200 μl 1-Step Ultra TMB (Thermo Scientific Fisher). Reactions were terminated with 100 μl TMB stop solution (KPL), and the absorbance immediately read at 450 nm using a Cytation 5 Imaging Reader (Biotek, Winooski, Vt.).

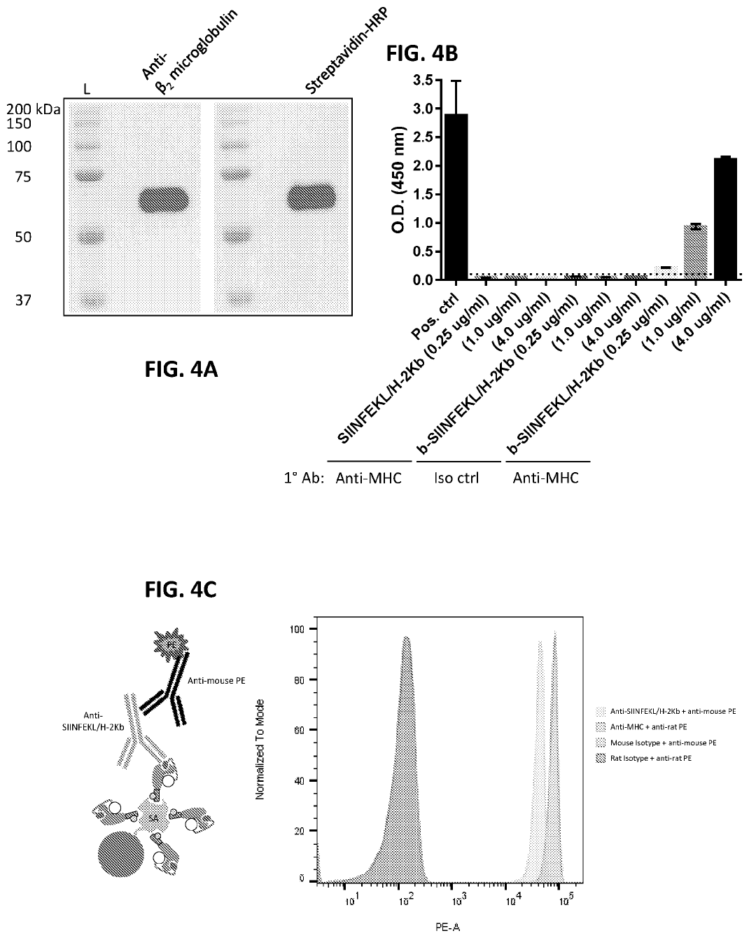

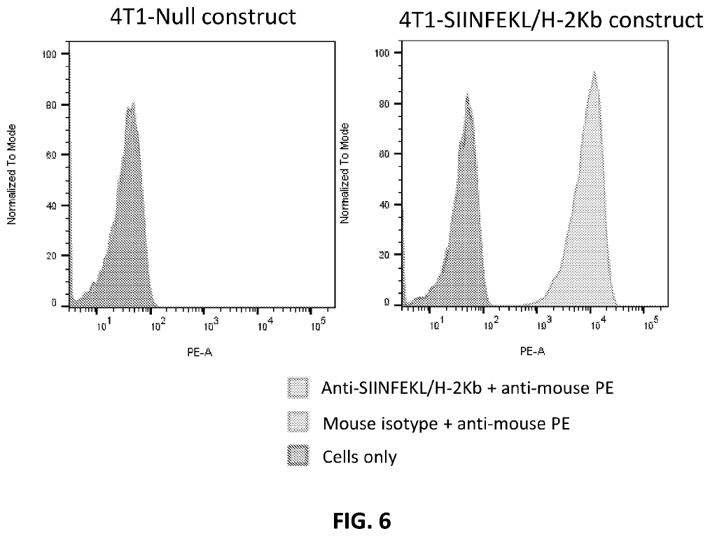

Flow cytometry. To confirm gene expression, the 4T1 cell line (1×10⁵ cells) was stained with relevant primary antibodies (anti-SIINFEKL/H-2 Kb or mouse IgG isotype antibodies at 2 μg/ml) in FACS buffer (0.5% BSA/0.1% NaN₃ in PBS) for 20 min at 4° C., washed, incubated with a PE-conjugated anti-mouse secondary antibody (Jackson ImmunoResearch), washed again, and resuspended in FACS buffer for analysis (FIG. 6). To ensure proper orientation, biotinylated peptide/MHC was incubated with 5 μm PMMA beads conjugated to streptavidin (PolyAn, Berlin, Germany), washed, and incubated at 4° C. with correct pairs of primary and PE-conjugated secondary antibodies (as indicated in FIGS. 4A to 4C) prior to analysis in FACS buffer. Frozen CD8+ T cells were thawed, washed, and resuspended in FACS buffer so that each stain consisted of 2×10⁵ viable CD8+ T cells. Cells were exposed to 50 nM dasatinib (Selleck Chemicals, Houston, Tex.) for 30 min at 37° C. and promptly incubated with peptide/MHC tetramers for 20 min at RT in the dark (Dolton et al., 2015). Cells were washed with FACS buffer and labeled with an anti-CD8-FITC antibody (clone 53-6.7; BioLegend, San Diego, Calif.) at 4° C. for 20 min shielded from light. After additional wash steps, cells were resuspended in fix solution (1% FBS, 2.5% formaldehyde in PBS) and assessed by flow cytometry using a BD LSRFortessa. Single and double color stain analysis was carried out using FlowJo software. In the case of tetramer analysis, PE/FITC compensated events were first gated based on FSC/SSC profiles and subsequently evaluated by PE and FITC fluorescence.

Cloning, expression, and purification strategy for soluble eukaryotic-derived peptide/MHC. The design of linked peptide/MHC class I molecules closely followed the previously reported generation of SCTs in bacteria (Hansen et al., 2009). Essentially, as presented in FIG. 1A, flexible glycine/serine linkers join a particular peptide epitope, β2 microglobulin, and MHC class I heavy chain. A BirA tail was also genetically encoded to the 3′ end of the molecule for enzyme-directed biotinylation of secreted protein. Yet, a notable difference of this design involved omitting the transmembrane region of MHC class I (designated sHeavychain) to allow for functional and fully-folded secreted protein. This process for eukaryotic expression centered on the use of a SB transposon system to stably integrate transgene content into relevant cell lines (FIG. 1B). Therefore, CHO cells would be transiently transfected with transposon-relevant plasmids (encoding separately a SB transposase and synthetic peptide/MHC molecule) and stable cells generated through antibiotic selection. After expanding relevant CHO cell clones, secreted peptide/MHC could be purified by AC, biotinylated, and multimerized to produce, for example, tetramers capable of binding antigen-specific T cells.

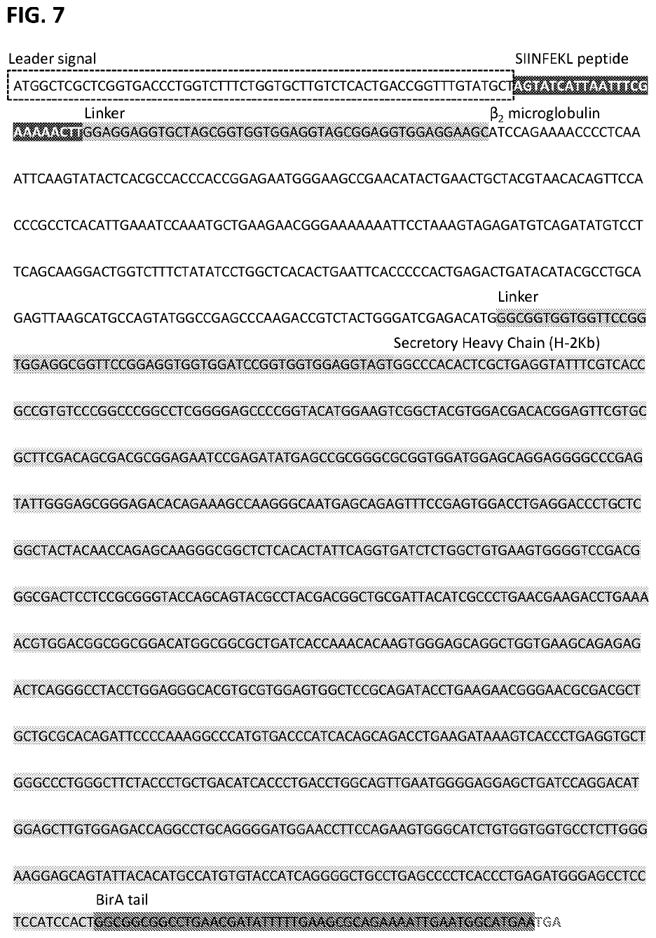

Purification of secreted and fully-folded peptide/MHC protein. The suitability of the SB transposon system to stably integrate the peptide/MHC transgene was first determined. However, this particular peptide/MHC construct differed from the aforementioned soluble design in FIG. 1A by encoding full-length MHC class I heavy chain (i.e., the murine MHC haplotype H-2 Kb), thereby, ensuring cell surface expression for relative ease of detection. Briefly, 4T1 (H-2 Kb null) cells were exposed to transposon vectors that expressed either a null or SIINFEKL/H-2 Kb construct and puromycin resistant clones selected and expanded in culture. Cells were then stained with isotype or anti-SIINFEKL/H-2 Kb antibodies and analyzed by flow cytometry using a PE-conjugated secondary antibody. In comparison to 4T1-null cells, 4T1-SIINFEKL/H-2 Kb cells expressed clear and robust levels of synthetic peptide/MHC (FIG. 7). These confirmatory results were expected, given the widespread use of the SB approach to induce expression of various protein classes in established cell lines and primary cells (Kebriaei et al., 2017).

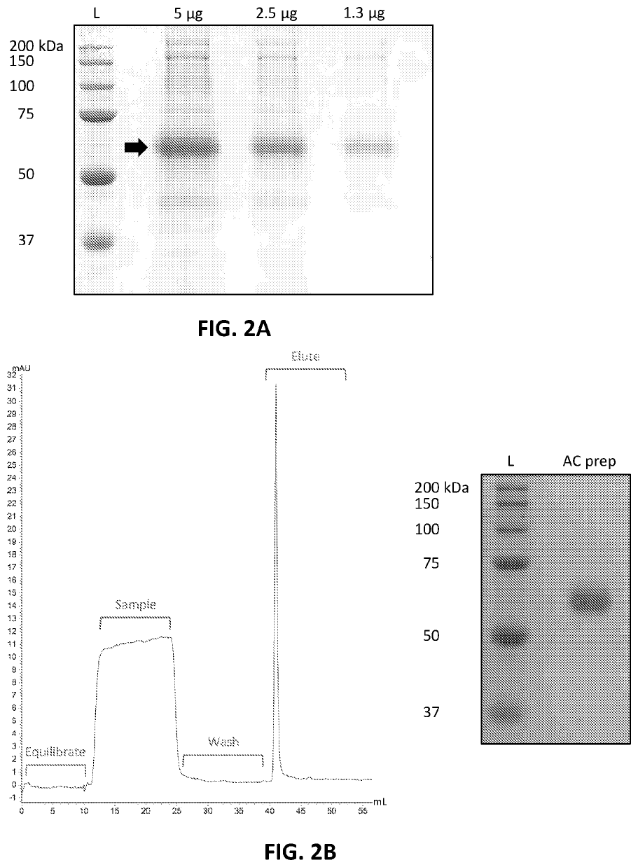

CHO cells were next transfected with SB-related vectors as described in FIGS. 1A and 1 B to induce stable expression of soluble peptide/MHC. Parental CHO cells exhibited enhanced resistance to puromycin, requiring sustained culturing in high concentrations of puromycin at 10 μg/ml. However, the inventors were able to expand puromycin-resistant clones by 2 weeks post plasmid transfection. Cells were then grown to saturating conditions in culture for at least 4 days to generate suitable whole protein concentrations from a total volume of 20 ml media. Yet, the choice of serum-free media contained a number of proprietary agents such as surfactants that could potentially interfere with protein purification and validation assays. The inventors, therefore, took the precautionary step of desalting and concentrating cell-free supernatants in PBS prior to downstream analysis. The initial assessment of CHO-derived extracellular proteins by SDS-PAGE (under reducing conditions) and coomassie blue staining revealed distinct protein bands around a predicted 51 kDa molecular weight for synthetic peptide/MHC (FIG. 2A) that was not evident from CHO cells expressing a null construct (data not shown). This specific protein band was subsequently confirmed following AC as exhibited in FIG. 2B. That is, cell-free supernatants were passed onto an agarose bead column containing the MHC class I-reactive M1/42 antibody and bound material eluted and ultimately assessed again by SDS-PAGE. The inventors typically harvested at least 100 μg/ml AC-purified peptide/MHC from small-scale culturing efforts. However, the described protocol can be scaled-up (or down) depending on the desired peptide/MHC total yield. Although beyond the scope of this report, alternative culturing parameters and/or leader signals (Haryadi et al., 2015) may potentially enhance overall CHO secretion of peptide/MHC. The AC procedure appeared to provide substantial protein purity based on obtaining [i] one distinct chromatogram elution peak and [ii] a protein band comprising >95% of detectable protein by coomassie blue staining.

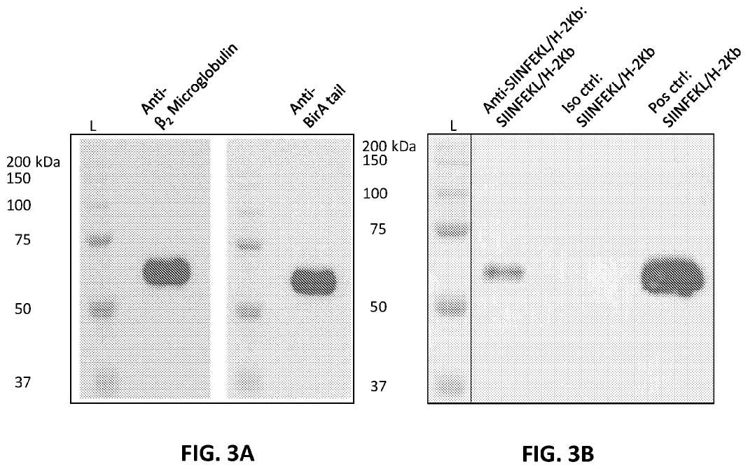

Determination of peptide/MHC identity and upstream binding potential. Next, the inventors confirmed the identity of AC purified protein by western blot separate from MHC class I reactivity. Based on the linked design of polychain peptide/MHC molecules (FIG. 1A), the inventors incorporated monoclonal antibodies specific to mouse 02 microglobulin and the BirA tail peptide GLNDIFEAQKIEWHE (SEQ ID NO: 600). Developed blots clearly verified the integrity of synthetic peptide/MHC molecule expression, indicating no observable issues in CHO cells secreting these artificial proteins (FIG. 3A). An immunoprecipitation reaction was subsequently attempted in order to demonstrate ligand binding potential of linked SIINFEKL to the MHC class I binding groove. SIINFEKL/H-2 Kb was incubated with 2 μg of anti-SIINFEKL/H-2 Kb or isotype control antibodies overnight. As detailed in the Materials and Methods, IgG containing material was purified using protein A/G-complexed agarose and resolved by SDS-PAGE under reducing conditions. The presence of SIINFEKL/H-2 Kb was further established through western blot using an anti-mouse 32 microglobulin antibody (FIG. 3B). Overall, these results help further validate the identity of soluble peptide/MHC from CHO cells and establish that these secreted proteins retain ligand binding specificity at the peptide/MHC class I interface.

Multimerization and functional assessment of eukaryotic-derived peptide/MHC. Biotinylated peptide/MHC arguably provides the greatest convenience to users for downstream assays. The inventors, therefore, designed a BirA tail to the terminal end of the peptide/MHC transgene by using a particular BirA tail peptide, GLNDIFEAQKIEWHE (SEQ ID NO: 600), which offers a highly targeted site for enzymatic conjugation of biotin with minimal footprint (Beckett et al., 1999). AC purified peptide/MHC was subjected to BirA ligase activity in the presence of free biotin overnight. Excess biotin and other reaction components were removed by excessive washing in PBS using a 30 MWCO centrifugal unit, and the extent of biotinylation first assessed by western blot and ELISA. In the case of western blot analysis, SDS-PAGE-resolved biotinylated protein was incubated with HRP-conjugated streptavidin to generate a specific peptide/MHC signal (FIG. 4A). Likewise, ELISA determination of biotinylated peptide/MHC clearly confirmed the functionality of the BirA tail region (FIG. 4B). Briefly, wells were coated overnight with streptavidin followed by various concentrations of peptide/MHC and biotinylated peptide/MHC. Detection was finally determined indirectly by MHC class I reactivity using the M1/42 antibody. Biotinylated SIINFEKL/H-2 Kb (as low as 0.25 μg/ml protein) was clearly evident in appropriate wells, with negligible reactivity occurring for conditions containing either unbiotinylated SIINFEKL/H-2 Kb or biotinylated SIINFEKL/H-2 Kb incubated with an isotype control. Next, the ability of immobilized/properly oriented biontinylated peptide/MHC to specifically engage ligands through the MHC class I peptide binding site was confirmed. Streptavidin was covalently attached to PMMA 5 μM beads and incubated with saturating conditions of biotinylated SIINFEKL/H-2 Kb. Beads were washed, incubated with appropriate primary reagents (i.e., anti-SIINFEKL/H-2 Kb and anti-MHC class I agents), and the extent of MHC ligand binding determined by flow cytometry using secondary PE-conjugated antibodies. Ultimately, as initially validated by the immunoprecipitation shown in FIG. 3B, biotinylated peptide/MHC bound streptavidin and specifically interacted with a TCR-like antibody that recapitulates CD8+ T cell interactions (FIG. 4C), indicating no observable functional issues with peptide/MHC post biotinylation and multimerization.

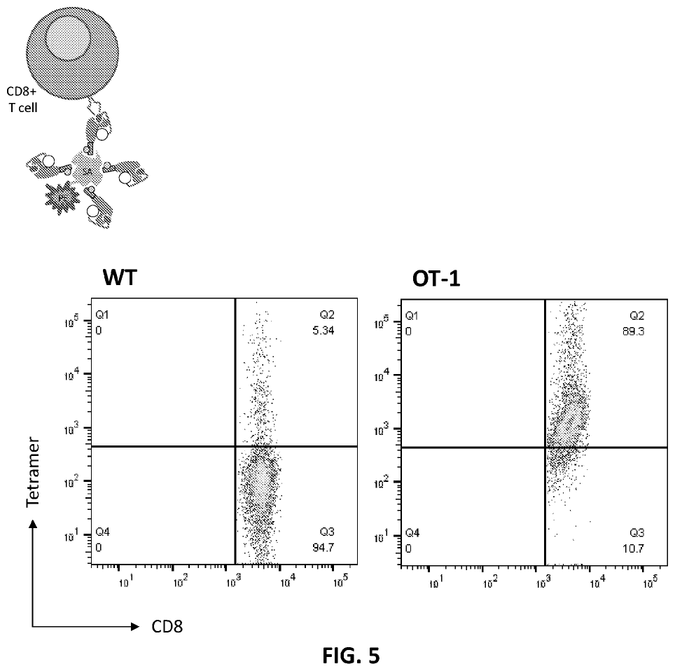

Next, the inventors assessed the ability of fluorescently-labeled multimers to specifically detect antigen-specific CD8+ T cells. Splenocytes and lymph nodes were harvested from wild-type and OT-1 transgenic (i.e., SIINFEKL-reactive) mice and CD8+ T cells subsequently purified through magnetic bead selection. Biotinylated SIINFEKL/H-2 Kb was then incubated with PE-conjugated streptavidin following a 4:1 molar ratio, thereby, generating tetramers. In order to stabilize membrane dynamics of TCRs, CD8+ T cells were exposed to dasatinib (as previously described [Dolton et al., 2015]), followed by incubation with tetramers. After suitable wash steps, cells were specifically labeled with a FITC-conjugated anti-mouse CD8 antibody that displays minimal interference with TCR:MHC binding (Clement et al., 2011). Cells were fixed and double-positive events (CD8+/tetramer+) assessed by flow cytometry. As detailed in FIG. 5, CD8+OT-1 cells clearly bound PE-labeled tetramers, with most cells displaying CD8 and tetramer positivity (in comparison to wild-type mice). Altogether, these data show the overarching strategy (as outlined in FIG. 1B) of stably producing eukaryotic-derived peptide/MHC that can be multimerized and used for immunologically-relevant assays such as CD8+ T cell identification.

Although MHC class I peptide candidates can be easily identified through in silico prediction methods (Andreatta and Nielsen 2016), free peptide occupancy of MHC class I molecules tends to be a rate limiting step in successfully generating stable peptide/MHC molecules from bacteria (Altman and Davis 2016). Considering the vital role MHC plays in human health (Cho and Sprent 2018), an inability to produce certain peptide/MHC reagents may adversely impact efforts on a number of fronts including diagnostics, therapies, and generalized scientific endeavors. For example, the burgeoning field of neoepitope identification for personalized cancer therapy could be stalled by those unique patient epitopes that exhibit high dissociation rates from MHC class I (Hu et al., 2018). One workaround to the issue of peptide occupancy has been in designing and expressing polychain SCTs that ensure full assembly of peptide, p2 microglobulin, and MHC class I heavy chain through flexible linkers (Hansen et al., 2009). SCTs retained on the plasma membrane of eukaryotic cells maintain their native conformation and are highly resistant to exogenous peptide binding (Yu et al., 2002). Additionally, membrane-bound SCTs serve as effective targets for CD8+ T cell priming (Hung et al., 2007) and destruction (Yu et al., 2002). In the case of diagnostic determination of CD8+ T cell frequencies by tetramers, SCTs can be modified for expression/secretion in bacteria and biotinylation by way of an explicit amino acid sequence that directs BirA enzyme function (Mitaksov et al., 2007).

Previously, the present inventors developed a unique protocol to establish eukaryotic cell lines (such as CHO suspension cells) in as little as two weeks that stably secrete peptide/MHC molecules through transposon-directed delivery. Soluble peptide/MHC may then be biotinylated and utilized as multimers (via streptavidin), particularly for CD8+ T cell relevant assays. Currently, CHO cells are an industry standard in producing FDA approved therapeutic recombinant proteins (Kuo et al., 2018). The inventors sought the advantages of the CHO cell line in order to [i] instigate post-translational modifications and [ii] be easily grown at high density under serum-free conditions in suspension cultures. However, other common cell line “protein workhorses” (e.g., HEK-293 cells) are amenable to the expression and characterization techniques outlined.

One advantage of the present invention is the rapid development of stable CHO cells secreting peptide/MHC using the SB transposon system. These studies incorporated the SB transposase SB100X, which has a high gene insertion efficiency at close-to-random chromosomal sites (Mates et al., 2009). The SB approach provides the advantageous properties of viral transduction to insert transgenes without the disadvantages of either maintaining genomic material episomally (e.g., adeno-associated viruses) or near/in proto-oncogenes (e.g., retroviral vectors) (Kebriaei et al., 2017). Additionally, manufacturing high-quality viral particles can be a cumbersome task fraught with regulatory obstacles. The inventors generally experienced minimal difficulty in developing and expanding stable lines from parental cells after transiently transfecting plasmids that propagated the SB transposon system. The protocol is also amenable to freezing material at convenient stopping points. There was no apparent adverse effects to generating multimerized reagents when either cell-free CHO-derived supernatants or biotinylated peptide/MHC was frozen long-term at −80° C. The method of the present invention is compatible with downstream tetramer production with the added benefits of convenient peptide/MHC expression that can incorporate a range of peptide affinities to the MHC peptide binding groove. Traditionally, to circumvent the low intrinsic affinity of the TCR with peptide/IIC, tetravalent multimers have been utilized to increase T cell avidity. However, these soluble peptide/MHC molecules can be utilized for higher order reagents to better discriminate low frequency T cells (or bind “difficult” TCRs) such as fluorochrome-conjugated dextramers since the production process involves incubating biotinylated peptide/MHC with a dextran backbone containing streptavidin (Dolton et al., 2014). Thus, the present invention can be used to reliably produce a range of soluble eukaryotic-derived peptide/MHC molecules for diagnostic, therapeutic, and investigative purposes.

TABLE 1
MHC, Peptide Sequences, and Target.

| ALLELE | PEPTIDE | TARGET | SEQ ID NO: |
|---|---|---|---|
| H-2 Db | AAVKNWMTQTL | SIV gag | 1 |
| H-2 Db | AGPHNDMEI | p56 | 2 |
| H-2 Db | AGVDNRECI | L1 | 3 |
| H-2 Db | AIQGNVTSI | Mycobacterium tuberculosis ESAT- | 4 |
| | | 6 | |
| H-2 Db | AQLANDVVL | MC-38 | 5 |
| H-2 Db | ASFRNLTHL | TPBG | 6 |
| H-2 Db | ASMTNMELM | MC38 adpgk neoantigen | 7 |
| H-2 Db | ASNENMDAM | NP | 8 |
| H-2 Db | ASNENMETM | NP | 9 |
| H-2 Db | ATFKNWPFL | Murine Survivin | 10 |
| H-2 Db | CMTWNQMNL | WT1 | 11 |
| H-2 Db | CSANNSHHYI | gp | 12 |
| H-2 Db | FQPQNGQFI | NP396 | 13 |
| H-2 Db | FSNSTNDILI | VEGFR2/KDR fragment 1 | 14 |
| H-2 Db | GAVQNEVTL | HCV NS3 | 15 |
| H-2 Db | HCIRNKSVI | PSA | 16 |
| H-2 Db | HCIRNKSVIL | hPSA | 17 |
| H-2 Db | HGIRNASFI | M45 | 18 |
| H-2 Db | KAVYNFATC | gP | 19 |
| H-2 Db | KAVYNFATM | gp33 (C9M) | 20 |
| H-2 Db | KCSRNRQYL | miHAg SMCY | 21 |
| H-2 Db | KVPRNQDWL | gp100 | 22 |
| H-2 Db | LGMSNRDFL | West Nile Virus polyprotein | 23 |
| H-2 Db | RAHYNIVTF | E7 | 24 |
| H-2 Db | RMFPNAPYL | WT1 | 25 |
| H-2 Db | RSPFSRVVHL | MOG precursor | 26 |
| H-2 Db | SCLENFRAYV | PolA | 27 |
| H-2 Db | SGPSNTPPEI | Ad5 E1A | 28 |
| H-2 Db | SGVENPGGYCL | gp | 29 |
| H-2 Db | SHLVEALYL | IGF | 30 |
| H-2 Db | SQLLNAKYL | Plasmodium berghei ANKA acid | 31 |
| | | phosphatase | |
| H-2 Db | SSLENFRAYV | PolA | 32 |
| H-2 Db | VILTNPISM | VEGFR2 | 33 |
| H-2 Db | WMHHNMDLI | H-Y | 34 |
| H-2 Dd | AGPHNDMEI | p56 | 35 |
| H-2 Dd | AGPPRYSRI | M164 | 36 |
| H-2 Dd | IGPGRAFYA | HIV-1 US4 gp120 | 37 |
| H-2 Dd | IPGRAFYA | Env | 38 |
| H-2 Dd | LGPISGHVL | pp65 | 39 |
| H-2 Dd | RGPGRAFVTI | HIV-1 IIIB gp120 | 40 |
| H-2 Dk | RRLGRTLLL | Middle T antigen | 41 |
| H-2 Kb | AGLAYYSM | Polyprotein | 42 |
| H-2 Kb | ANYNFTLV | Trypanosoma cruzi SP | 43 |
| H-2 Kb | ATLTYRML | Yellow Fever Virus 17D | 44 |
| | | polyprotein | |
| H-2 Kb | DAPIYTNV | Beta-gal | 45 |
| H-2 Kb | EVYDFAFRDL | E6 | 46 |
| H-2 Kb | FAPGNYPAL | NP | 47 |
| H-2 Kb | HILIYSDV | MYBPC-2 | 48 |
| H-2 Kb | HNTQYCNL | MAGE-A5 | 49 |
| H-2 Kb | ICPMYARV | Beta-gal | 50 |
| H-2 Kb | ILSPFLPLL | HBV surface antigen | 51 |
| H-2 Kb | IMYNYPAM | TB10.3-4 | 52 |
| H-2 Kb | IMYNYPAML | Mycobacterium tuberculosis | 53 |
| | | TB10.4 | |
| H-2 Kb | ISHNFCNL | gp | 54 |
| H-2 Kb | KSPWFTTL | MuLV env | 55 |
| H-2 Kb | KVVRFDKL | Ovalbumin (subdominant) | 56 |
| H-2 Kb | LTFNYRNL | Histocompatibility antigen 60 | 57 |
| H-2 Kb | MGLKFRQL | CP | 58 |
| H-2 Kb | RGYVYQGL | NP52 | 59 |
| H-2 Kb | SCLEFWQRV | M57 | 60 |
| H-2 Kb | SDYYFSWL | muFAP α | 61 |
| H-2 Kb | SIINFEKL | OVA | 62 |
| H-2 Kb | SIIVFNLL | MC-38 | 63 |
| H-2 Kb | SIYRYYGL | OVA | 64 |
| H-2 Kb | SKYVFENV | MYBPC-2 | 65 |
| H-2 Kb | SSIEFARL | gp | 66 |
| H-2 Kb | STYTFVRT | M38 | 67 |
| H-2 Kb | SVLAFRRL | tgd057 | 68 |
| H-2 Kb | SVYDFFVWL | TRP2 | 69 |
| H-2 Kb | TSINFVKI | P79 | 70 |
| H-2 Kb | TSYKFESV | INF γ R | 71 |
| H-2 Kb | TVSEFLKL | Murine Survivin | 72 |
| H-2 Kb | TVYGFCLL | m139 | 73 |
| H-2 Kb | VGRNFTNL | mTERT | 74 |
| H-2 Kb | VIDAFSRL | m141 | 75 |
| H-2 Kb | VIYIFTVRL | VACCL3_100 | 76 |
| H-2 Kb | VNHRFTLV | Trypanosoma cruzi ASP-2 | 77 |
| H-2 Kb | VVYDFLKL | SV40 T antigen | 78 |
| H-2 Kb | VWLSVIWM | HBsAg | 79 |
| H-2 Kd | AMQMLKDTI | HIV gag p24 | 80 |
| H-2 Kd | AMQMLKETI | HIV-1 gag p24 | 81 |
| H-2 Kd | AYIDFEMKI | SART3 | 82 |
| H-2 Kd | CYYASRTKL | m145 | 83 |
| H-2 Kd | DYWGQGTEL | Surface IgG (sA20-Ig) of A20 | 84 |
| H-2 Kd | EYILSLEEL | GPC3 | 85 |
| H-2 Kd | GYETVITQL | PPE | 86 |
| H-2 Kd | GYKDGNEYI | Listeria monocytogenes | 87 |
| | | Listeriolysin | |
| H-2 Kd | HYLSTQSAL | Enhanced Green Fluorescent | 88 |
| | | Protein (eGFP) | |
| H-2 Kd | IYNVGQVSI | Sialidases | 89 |
| H-2 Kd | IYSTVASSL | ND | 90 |
| H-2 Kd | KYKNAVTEL | RSV A strain F protein | 91 |
| H-2 Kd | KYNKANVFL | NRP-V7 superagonist peptide 8.3 | 92 |
| | | Tg NOD mouse | |
| H-2 Kd | QYIHSANVL | Erk1 | 93 |
| H-2 Kd | RYLKNGKETL | HLA-Cw3 | 94 |
| H-2 Kd | SYIGSINNI | M2-1 | 95 |
| H-2 Kd | SYIPSAEKI | Plasmodium berghei CSP | 96 |
| H-2 Kd | SYMLQALCI | TNP03 | 97 |
| H-2 Kd | SYVPSAEQI | Plasmodium CSP | 98 |
| H-2 Kd | TYLPTNASL | HER2 | 99 |
| H-2 Kd | TYQRTRALV | Influenza A (PR8) NP | 100 |
| H-2 Kd | TYVPANASL | Neu/Her-2/Erbb2 proto- | 101 |
| | | oncoprotein | |
| H-2 Kd | TYWPVVSDI | UL105 | 102 |
| H-2 Kd | VYAGAMSGL | Mtb85A | 103 |
| H-2 Kd | YYIPHQSSL | Plasmodium falciparum Liver | 104 |
| | | stage antigen | |
| H-2 Kk | DYENDIEKKI | CPS | 105 |
| H-2 Kk | FETFEAKI | Tyrosine-3-hydroxylase | 106 |
| H-2 Kk | TENSGKDI | SMCY | 107 |
| H-2 Kk | TEWETGQI | ASP-2 | 108 |
| H-2 Kk | VESTAGSL | ESAT-6 | 109 |
| H-2 Kk | YENDIEKKI | PLAM csp | 110 |
| H-2 Ld | HPQKVTKFM | KLK3 | 111 |
| H-2 Ld | IPQSLDSWWTSL | HBV surface antigen | 112 |
| H-2 Ld | LPYLGWLVF | P1A | 113 |
| H-2 Ld | MPVGGQSSF | Ag85A | 114 |
| H-2 Ld | MPYLIDFGL | VSV N | 115 |
| H-2 Ld | RPQASGVYM | NP | 116 |
| H-2 Ld | SPGAAGYDL | Dutpase | 117 |
| H-2 Ld | SPSYVYHQF | EMV-1 | 118 |
| H-2 Ld | TPHPARIGL | Beta-gal | 119 |
| H-2 Ld | YPHFMPTNL | MCMV IE1 | 120 |
| HLA-A*0101 | ATDALMTGF | HCV NS3 | 121 |
| HLA-A*0101 | ATDALMTGY | Polyprotein | 122 |
| HLA-A*0101 | CTELKLSDY | NP | 123 |
| HLA-A*0101 | EADPTGHSY | MAGE-1 | 124 |
| HLA-A*0101 | EVDPIGHLY | MAGE-3 | 125 |
| HLA-A*0101 | FTELTLGEF | Survivin | 126 |
| HLA-A*0101 | IVDCLTEMY | USP9Y | 127 |
| HLA-A*0101 | KSDICTDEY | Tyrosinase | 128 |
| HLA-A*0101 | SADNNNSEY | VP1 | 129 |
| HLA-A*0101 | SSDYVIPIGTY | Tyrosinase | 130 |
| HLA-A*0101 | TDLGQNLLY | AdV 5 Hexon | 131 |
| HLA-A*0101 | TLDTLTAFY | MSLN | 132 |
| HLA-A*0101 | TSEKRPFMCAY | WT1 | 133 |
| HLA-A*0101 | VSDGGPNLY | Influenza A PB1 | 134 |
| HLA-A*0101 | VTEHDTLLY | UL44 | 135 |
| HLA-A*0101 | YSEHPTFTSQY | HCMV pp65 | 136 |
| HLA-A*0201 | AILALLPAL | PSCA | 137 |
| HLA-A*0201 | AIQDLCLAV | NPM1 | 138 |
| HLA-A*0201 | AIQDLCVAV | NPM1 | 139 |
| HLA-A*0201 | AITEVECFL | VP1 | 140 |
| HLA-A*0201 | ALCNTDSPL | iLR1 | 141 |
| HLA-A*0201 | ALDVYNGLL | ACPP | 142 |
| HLA-A*0201 | ALFDIESKV | PSM P2 | 143 |
| HLA-A*0201 | ALIAPVHAV | Neg. Control | 144 |
| HLA-A*0201 | ALISAFSGS | K8.1 | 145 |
| HLA-A*0201 | ALKDVEERV | MAGE-C2 | 146 |
| HLA-A*0201 | ALLEIASCL | ID0 | 147 |
| HLA-A*0201 | ALLTSRLRFI | Telomerase | 148 |
| HLA-A*0201 | ALMEQQHYV | ITGB8 | 149 |
| HLA-A*0201 | ALNVYNGLL | ACPP | 150 |
| HLA-A*0201 | ALPFGFILV | IL13Ra | 151 |
| HLA-A*0201 | ALPHIIDEV | ND | 152 |
| HLA-A*0201 | ALQPGTALL | PSCA | 153 |
| HLA-A*0201 | ALSPVPPVV | BCL-2 | 154 |
| HLA-A*0201 | ALTPVVVTL | cyclin-dependent kinase 4 | 155 |
| HLA-A*0201 | ALVCYGPGI | FAP α | 156 |
| HLA-A*0201 | ALVEMGHHA | Vpu | 157 |
| HLA-A*0201 | ALWALPHAA | IE62 | 158 |
| HLA-A*0201 | ALWGPDPAAA | Insulin | 159 |
| HLA-A*0201 | ALWPWLLMAT | RNF43 | 160 |
| HLA-A*0201 | ALYDVVTKL | Polyprotein | 161 |
| HLA-A*0201 | ALYLMELTM | CB9L2 | 162 |
| HLA-A*0201 | ALYVDSLFFL | PRAME | 163 |
| HLA-A*0201 | AMASTEGNV | ESAT-6 | 164 |
| HLA-A*0201 | AMLVLLAEI | LANA | 165 |
| HLA-A*0201 | AQCQETIRV | Midkine | 166 |
| HLA-A*0201 | ATGEALWAL | IE62 | 167 |
| HLA-A*0201 | ATWAENIQV | West Nile virus NY-99 | 168 |
| | | polyprotein precursor | |
| HLA-A*0201 | AVLDGLLSL | bZIP factor | 169 |
| HLA-A*0201 | CINGVCWTV | NS3 | 170 |
| HLA-A*0201 | CLGGLLTMV | LMP-2A | 171 |
| HLA-A*0201 | CLPSPSTPV | BMI1 | 172 |
| HLA-A*0201 | CLWCVPQLR | ABL1 | 173 |
| HLA-A*0201 | CVNGVCWTV | Polyprotein | 174 |
| HLA-A*0201 | DIWDGIPHV | NRP-2 | 175 |
| HLA-A*0201 | DLMGYIPAV | HCV core | 176 |
| HLA-A*0201 | DLMGYIPLV | HCV core | 177 |
| HLA-A*0201 | EIWTHSYKV | FOLR1 | 178 |
| HLA-A*0201 | ELAGIGILTV | MART-1 | 179 |
| HLA-A*0201 | ELSDSLGPV | PASD1 | 180 |
| HLA-A*0201 | ELTLGEFLKL | Survivin | 181 |
| HLA-A*0201 | ELVDGLLSL | bZIP factor | 182 |
| HLA-A*0201 | FIDSYICQV | H-Y | 183 |
| HLA-A*0201 | FILGIIITV | V131 | 184 |
| HLA-A*0201 | FIYDFCIFGV | Lengsin | 185 |
| HLA-A*0201 | FLAEDALNTV | EDDR1 | 186 |
| HLA-A*0201 | FLAMLKNTV | MAGE-C1 | 187 |
| HLA-A*0201 | FLDKGTYTL | BALF4 | 188 |
| HLA-A*0201 | FLDPRPLTV | CYP190 | 189 |
| HLA-A*0201 | FLFLRNFSL | TARP | 190 |
| HLA-A*0201 | FLGKIWPS | Gag | 191 |
| HLA-A*0201 | FLGYLILGV | PAP-3 | 192 |
| HLA-A*0201 | FLLSLFSLWL | ZnT-8 | 193 |
| HLA-A*0201 | FLLSLGIHL | HBV polymerase | 194 |
| HLA-A*0201 | FLLTRILTI | S protein | 195 |
| HLA-A*0201 | FLNKCETWV | DLK1 | 196 |
| HLA-A*0201 | FLPSDFFPSI | HBV core | 197 |
| HLA-A*0201 | FLPSDFFPSV | CP | 198 |
| HLA-A*0201 | FLPSPLFFFL | TARP 2M | 199 |
| HLA-A*0201 | FLTPKKLQCV | PSA | 200 |
| HLA-A*0201 | FLWGPRALV | MAGE-3 | 201 |
| HLA-A*0201 | FLYALALLL | LMP-2A | 202 |
| HLA-A*0201 | FLYDDNQRV | topII | 203 |
| HLA-A*0201 | FMNKFIYEI | alfa fetoprotein | 204 |
| HLA-A*0201 | FVGEFFTDV | Glypican 3 | 205 |
| HLA-A*0201 | GILGFVFTL | MP | 206 |
| HLA-A*0201 | GILTVSVAV | Mtb 16 kDa | 207 |
| HLA-A*0201 | GLADQLIHL | Vif | 208 |
| HLA-A*0201 | GLAPPQHLIRV | p53 | 209 |
| HLA-A*0201 | GLCTLVAML | BMLF1 | 210 |
| HLA-A*0201 | GLFKCGIAV | FAP α | 211 |
| HLA-A*0201 | GLIQLVEGV | TRAG | 212 |
| HLA-A*0201 | GLLGASVLGL | Telomerase | 213 |
| HLA-A*0201 | GLLRFVTAV | NRP-1 | 214 |
| HLA-A*0201 | GLLSLEEEL | bZIP factor | 215 |
| HLA-A*0201 | GLMEEMSAL | Mena | 216 |
| HLA-A*0201 | GLPVEYLQV | Mycobacterium bovis antigen 85-A | 217 |
| HLA-A*0201 | GLQDCTMLV | HCV NS5B | 218 |
| HLA-A*0201 | GLQHWVPEL | BA46 | 219 |
| HLA-A*0201 | GLSPTVWLSV | S protein | 220 |
| HLA-A*0201 | GLSRYVARL | Polymerase | 221 |
| HLA-A*0201 | GLYDGMEHL | MAGE-10 | 222 |
| HLA-A*0201 | GMLGMVSGL | NRP-1 | 223 |
| HLA-A*0201 | GVDPNIRTGV | Polyprotein | 224 |
| HLA-A*0201 | GVLVGVALI | CEA | 225 |
| HLA-A*0201 | GVRGRVEEI | BCR-ABL | 226 |
| HLA-A*0201 | GVYDGREHTV | MAGE-4 | 227 |
| HLA-A*0201 | HIAGSLAVV | ZnT-8 | 228 |
| HLA-A*0201 | HLSTAFARV | G250 | 229 |
| HLA-A*0201 | HLVEALYLV | Insulin | 230 |
| HLA-A*0201 | ILAKFLHWL | Telomerase | 231 |
| HLA-A*0201 | ILDDNLYKV | Vaccinia virus Copenhagen | 232 |
| | | Protein G5 | |
| HLA-A*0201 | ILGFVFTLTV | MP | 233 |
| HLA-A*0201 | ILGVLTSLV | DLK1 | 234 |
| HLA-A*0201 | ILHDGAYSL | HER2 | 235 |
| HLA-A*0201 | ILHNGAYSL | HER2 | 236 |
| HLA-A*0201 | ILKDFSILL | ZnT-8 | 237 |
| HLA-A*0201 | ILKEPVHGV | RT | 238 |
| HLA-A*0201 | ILLWEIFTL | PDGFRbeta | 239 |
| HLA-A*0201 | ILLWQPIPV | PAP-3 | 240 |
| HLA-A*0201 | ILMWEAVTL | VPI | 241 |
| HLA-A*0201 | ILSLELMKL | HMMR | 242 |
| HLA-A*0201 | IMDQVPFSV | gp100 | 243 |
| HLA-A*0201 | ITDQVPFSV | gp100 | 244 |
| HLA-A*0201 | KIFGSLAFL | HER2 | 245 |
| HLA-A*0201 | KLCPVQLWV | p53 | 246 |
| HLA-A*0201 | KLDVGNAEV | BAP31 | 247 |
| HLA-A*0201 | KLFGTSGQKT | EGFR | 248 |
| HLA-A*0201 | KLGEFYNQMM | BNP | 249 |
| HLA-A*0201 | KLHLYSHPI | Polymerase | 250 |
| HLA-A*0201 | KLIANNTRV | Mycobacterium bovis antigen 85-A | 251 |
| HLA-A*0201 | KLMSSNSTDL | HSP105 | 252 |
| HLA-A*0201 | KLPQLCTEL | HPV 16 E6 | 253 |
| HLA-A*0201 | KLQCVDLHV | PSA | 254 |
| HLA-A*0201 | KLQDASAEV | HM1.24 | 255 |
| HLA-A*0201 | KLQVFLIVL | IAPP | 256 |
| HLA-A*0201 | KLSGLGINAV | HCV NS3 | 257 |
| HLA-A*0201 | KLTPLCVTL | HIV-1 env gp120 848-856 | 258 |
| HLA-A*0201 | KLVALGINAV | Polyprotein | 259 |
| HLA-A*0201 | KMLKEMGEV | RSV NP | 260 |
| HLA-A*0201 | KTWGQYWQV | gp100 | 261 |
| HLA-A*0201 | KVAEELVHFL | MAGE-A3 | 262 |
| HLA-A*0201 | KVAELVHFL | MAGE-A3 | 263 |
| HLA-A*0201 | KVDDTFYYV | Vaccinia virus Host range | 264 |
| | | protein 2 | |
| HLA-A*0201 | KVLEYVIKV | MAGE-A1 | 265 |
| HLA-A*0201 | KVVEFLAML | MAGE-C1 | 266 |
| HLA-A*0201 | LAALPHSCL | RGS5 | 267 |
| HLA-A*0201 | LIAHNQVRQV | HER2 | 268 |
| HLA-A*0201 | LIDQYLYYL | VP1 | 269 |
| HLA-A*0201 | LILPLLFYL | NG2 | 270 |
| HLA-A*0201 | LLAARAIVAI | Ilr1 | 271 |
| HLA-A*0201 | LLDFVRFMGV | EBNA 3B | 272 |
| HLA-A*0201 | LLDVPTAAV | Interferon gamma inducible | 273 |
| | | protein (GILT) 30 | |
| HLA-A*0201 | LLFGLALIEV | MAGE-C2 | 274 |
| HLA-A*0201 | LLFGYPVYV | Tax | 275 |
| HLA-A*0201 | LLFNILGGWV | HCV NS4b | 276 |
| HLA-A*0201 | LLGRNSFEV | p53 | 277 |
| HLA-A*0201 | LLHETDSAV | PSMA | 278 |
| HLA-A*0201 | LLLASIAAGL | LY6K | 279 |
| HLA-A*0201 | LLLGPLGPL | Heparanase | 280 |
| HLA-A*0201 | LLLIWFRPV | Large T antigen | 281 |
| HLA-A*0201 | LLLLTVLTV | Mucin | 282 |
| HLA-A*0201 | LLLNCLWSV | Spike GP | 283 |
| HLA-A*0201 | LLLTVLTVV | Mucin | 284 |
| HLA-A*0201 | LLMGTLGIVC | E7 | 285 |
| HLA-A*0201 | LLMWEAVTV | VP1 | 286 |
| HLA-A*0201 | LLNGWRWRL | ND | 287 |
| HLA-A*0201 | LLQERGVAYI | PSMA | 288 |
| HLA-A*0201 | LLTAALWYV | CD105 | 289 |
| HLA-A*0201 | LLVPTCVFLV | TEM1 | 290 |
| HLA-A*0201 | LLWNGPMAV | Polyprotein | 291 |
| HLA-A*0201 | LMLGEFLKL | Survivin | 292 |
| HLA-A*0201 | LMWYELSKI | gp | 293 |
| HLA-A*0201 | LNIDLLWSV | IGRP | 294 |
| HLA-A*0201 | LTFGWCFKL | HIV-1 Nef | 295 |
| HLA-A*0201 | LTLGEFLKL | Survivin-3a | 296 |
| HLA-A*0201 | LVWMACHSA | Nucleocapsid | 297 |
| HLA-A*0201 | MLAVFLPIV | STEAP1 | 298 |
| HLA-A*0201 | MLDLQPETT | E7 | 299 |
| HLA-A*0201 | MLMAQEALAFL | CAMEL | 300 |
| HLA-A*0201 | MLNIPSINV | pp65 | 301 |
| HLA-A*0201 | MMNDQLMFL | PSMA | 302 |
| HLA-A*0201 | MVWESGCTV | IA-2 | 303 |
| HLA-A*0201 | NLFETPVEA | BA46 | 304 |
| HLA-A*0201 | NLVPMVATV | pp65 | 305 |
| HLA-A*0201 | NVWATHACV | Env | 306 |
| HLA-A*0201 | PLFDFSWLSL | BCL-2 | 307 |
| HLA-A*0201 | QLCPICRAPV | LIVIN | 308 |
| HLA-A*0201 | QLFEELQEL | H0-1 | 309 |
| HLA-A*0201 | QLFNHTMFI | Non-muscle Myosin | 310 |
| HLA-A*0201 | QLGEQCWTV | PSCA | 311 |
| HLA-A*0201 | QLLDGFMITL | PASD1 | 312 |
| HLA-A*0201 | QLLIKAVNL | MPP11 | 313 |
| HLA-A*0201 | QMARLAWEA | LANA | 314 |
| HLA-A*0201 | QQAHCLWCV | ABL1 | 315 |
| HLA-A*0201 | RILGAVAKV | Vinculin | 316 |
| HLA-A*0201 | RLAEYQAYI | SART3 | 317 |
| HLA-A*0201 | RLDDDGNFQL | West Nile Virus NY-99 | 318 |
| | | polyprotein precursor | |
| HLA-A*0201 | RLLGNVLVCV | HBB | 319 |
| HLA-A*0201 | RLLQETELV | HER2 | 320 |
| HLA-A*0201 | RLLVVYPWT | HBB | 321 |
| HLA-A*0201 | RLMNDMTAV | HSP105 | 322 |
| HLA-A*0201 | RLNMFTPYI | chlamydia trachomatis MOMP | 323 |
| HLA-A*0201 | RLSSCVPVA | TGF β | 324 |
| HLA-A*0201 | RLTPGVHEL | DLK1 | 325 |
| HLA-A*0201 | RLTSRVKAL | Telomerase | 326 |
| HLA-A*0201 | RLVDDFLLV | Telomerase | 327 |
| HLA-A*0201 | RLWQELSDI | circadian clock protein PASD1 | 328 |
| HLA-A*0201 | RMFPNAPYL | WT1 | 329 |
| HLA-A*0201 | RMPEAAPPV | p53 | 330 |
| HLA-A*0201 | RTLDKVLEV | HA-8 | 331 |
| HLA-A*0201 | RTLNAWVKV | Gag | 332 |
| HLA-A*0201 | RVASPTSGV | IRS-2 | 333 |
| HLA-A*0201 | SIDWFMVTV | PLAC1 | 334 |
| HLA-A*0201 | SILLRDAGLV | TRAG | 335 |
| HLA-A*0201 | SITEVECFL | VPI | 336 |
| HLA-A*0201 | SLFEPPPPG | PSMA | 337 |
| HLA-A*0201 | SLFLGILSV | MS4A1 | 338 |
| HLA-A*0201 | SLFNTVATL | Gag | 339 |
| HLA-A*0201 | SLFNTVATLY | Gag | 340 |
| HLA-A*0201 | SLGEQQYSV | WT1 | 341 |
| HLA-A*0201 | SLLFLLFSL | MSLN | 342 |
| HLA-A*0201 | SLLMWITQC | NY-ESO-1 | 343 |
| HLA-A*0201 | SLLMWITQV | NY-ESO-1 | 344 |
| HLA-A*0201 | SLLNATAIAV | Env | 345 |
| HLA-A*0201 | SLLQHLIGL | PRAME | 346 |
| HLA-A*0201 | SLNQTVHSL | ND | 347 |
| HLA-A*0201 | SLPPPGTRV | p53 | 348 |
| HLA-A*0201 | SLSEKTVLL | CD59 | 349 |
| HLA-A*0201 | SLSRFSWGA | Myelin basic protein | 350 |
| HLA-A*0201 | SLVDVMPWL | Cytochrome p450 | 351 |
| HLA-A*0201 | SLVKHHMYI | Vif | 352 |
| HLA-A*0201 | SLYNTVATL | Gag | 353 |
| HLA-A*0201 | SLYNTVATLY | Gag | 354 |
| HLA-A*0201 | SLYSFPEPEA | PRAME | 355 |
| HLA-A*0201 | SMYRVFEVGV | H250 | 356 |
| HLA-A*0201 | SQADALKYV | EZH2 | 357 |
| HLA-A*0201 | STLCQVEPV | MPP11 | 358 |
| HLA-A*0201 | STPPPGTRV | p53 | 359 |
| HLA-A*0201 | TLADFDPRV | EphA2 | 360 |
| HLA-A*0201 | TLDYKPLSV | BMRF1 | 361 |
| HLA-A*0201 | TLFWLLLTL | VEGFR1 | 362 |
| HLA-A*0201 | TLFWLLTL | FLT1 | 363 |
| HLA-A*0201 | TLNAWVKVV | Gag | 364 |
| HLA-A*0201 | TLPGYPPHV | PAX-5 | 365 |
| HLA-A*0201 | TLPPAWQPFL | Survivin | 366 |
| HLA-A*0201 | TLQDIVYKL | BMI1 | 367 |
| HLA-A*0201 | TLSNLSFPV | NG2 | 368 |
| HLA-A*0201 | TMNGSKSPV | Mena | 369 |
| HLA-A*0201 | VIFDFLHCI | Large T antigen | 370 |
| HLA-A*0201 | VIMPCSWWV | Chondromodulin | 371 |
| HLA-A*0201 | VISNDVCAQV | KLK | 372 |
| HLA-A*0201 | VIVMLTPLV | IA-2 | 373 |
| HLA-A*0201 | VIYHYVDDL | Pol | 374 |
| HLA-A*0201 | VLAELVKQI | HCMV IE1 | 375 |
| HLA-A*0201 | VLAGGFFLL | PSMA | 376 |
| HLA-A*0201 | VLDFAPPGA | WT1 | 377 |
| HLA-A*0201 | VLDGLDVLL | PRAME | 378 |
| HLA-A*0201 | VLEETSVML | IE-1 | 379 |
| HLA-A*0201 | VLFGLGFAI | IGRP | 380 |
| HLA-A*0201 | VLHDDLLEA | HA-1 | 381 |
| HLA-A*0201 | VLLGAVCGV | NRP-1 | 382 |
| HLA-A*0201 | VLMIKALEL | Non muscle Myosin-9 | 383 |
| HLA-A*0201 | VLPLTVAEV | MSLN | 384 |
| HLA-A*0201 | VLQELNVTV | Pr1 | 385 |
| HLA-A*0201 | VLQMKEEDV | iLR1 | 386 |
| HLA-A*0201 | VLSDFKTWL | HCV NS5a | 387 |
| HLA-A*0201 | VLTDGNPPEV | Mtb 19 kDa | 388 |
| HLA-A*0201 | VLYRYGSFSV | gp100 | 389 |
| HLA-A*0201 | VMNILLQYVV | GAD65 | 390 |
| HLA-A*0201 | VVTGVLVYL | ZnT-8 | 391 |
| HLA-A*0201 | WLSLKTLLSL | BCL-2 | 392 |
| HLA-A*0201 | WLSLLVPFV | S protein | 393 |
| HLA-A*0201 | YAYDGKDYIA | HLA-A2 | 394 |
| HLA-A*0201 | YIGEVLVSV | HA-2 | 395 |
| HLA-A*0201 | YLEPGPVTA | gp100 | 396 |
| HLA-A*0201 | YLEPGPVTV | gp100 | 397 |
| HLA-A*0201 | YLFFYRKSV | mTERT | 398 |
| HLA-A*0201 | YLGSYGFRL | p53 | 399 |
| HLA-A*0201 | YLIELIDRV | TACE | 400 |
| HLA-A*0201 | YLISGDSPV | CD33 | 401 |
| HLA-A*0201 | YLLEMLWRL | LMP-1 | 402 |
| HLA-A*0201 | YLLPRRGPRL | HCV core | 403 |
| HLA-A*0201 | YLNDHLEPWI | BCL-X | 404 |
| HLA-A*0201 | YLNKIQNSL | Plasmodium falciparum CSP | 405 |
| HLA-A*0201 | YLNRHLHTWI | BCL-2 | 406 |
| HLA-A*0201 | YLNTVQPTCV | EGFR | 407 |
| HLA-A*0201 | YLQLVFGIEV | MAGE-A2 | 408 |
| HLA-A*0201 | YLQQNWWTL | LMP-1 | 409 |
| HLA-A*0201 | YLQQNWWTL | LMP-1 | 410 |
| HLA-A*0201 | YLQVDLRFL | NRP-2 | 411 |
| HLA-A*0201 | YLQVNSLQTV | Telomerase | 412 |
| HLA-A*0201 | YLQWIEFSI | Prominin1 | 413 |
| HLA-A*0201 | YLSGADLNL | CEA | 414 |
| HLA-A*0201 | YLSGANLNL | CEACAM | 415 |
| HLA-A*0201 | YLVGNVCIL | PASD1 | 416 |
| HLA-A*0201 | YLVSIFLHL | ND | 417 |
| HLA-A*0201 | YLYQWLGAPV | BGLAP | 418 |
| HLA-A*0201 | YMCSFLFNL | EZH2 | 419 |
| HLA-A*0201 | YMDGTMSQV | Tyrosinase | 420 |
| HLA-A*0201 | YMLDLQPETT | E7 | 421 |
| HLA-A*0201 | YMNGTMSQV | Tyrosinase | 422 |
| HLA-A*0201 | YTCPLCRAPV | SAA | 423 |
| HLA-A*0201 | YTMDGEYRL | West Nile virus NY-99 | 424 |
| | | polyprotein precursoR | |
| HLA-A*0201 | YVLDHLIVV | BRLF1 | 425 |
| HLA-A*0301 | AIFQSSMTK | HIV pol | 426 |
| HLA-A*0301 | ALLAVGATK | gp100 | 427 |
| HLA-A*0301 | ATGFKQSSK | bcr-abl 210 kD fusion protein | 428 |
| HLA-A*0301 | ILRGSVAHK | Influenza A (PR8) NP | 429 |
| HLA-A*0301 | KLCLRFLSK | E6 | 430 |
| HLA-A*0301 | KLGGALQAK | IE-1 | 431 |
| HLA-A*0301 | KQSSKALQR | bcr-abl 210 kD fusion protein | 432 |
| HLA-A*0301 | QVLKKIAQK | HMOX1 | 433 |
| HLA-A*0301 | QVPLRPMTYK | NEF | 434 |
| HLA-A*0301 | RIAAWMATY | BCL-2L1 | 435 |
| HLA-A*0301 | RISTFKNWPK | Survivin-3a | 436 |
| HLA-A*0301 | RLGLQVRKNK | RhoC | 437 |
| HLA-A*0301 | RLLFFAPTR | MCL-1 | 438 |
| HLA-A*0301 | RLRAEAQVK | EMNA 3A | 439 |
| HLA-A*0301 | RLRPGGKKK | Gag | 440 |
| HLA-A*0301 | RVCEKMALY | HCV NS5B | 441 |
| HLA-A*0301 | RVRAYTYSK | BRLF1 | 442 |
| HLA-A*0301 | SVLNYERARR | hTERT | 443 |
| HLA-A*0301 | VTLTHPITK | Polyprotein | 444 |
| HLA-A*0301 | YMVPFIPLYR | Tyrosinase | 445 |
| HLA-A*1101 | ACQGVGGPGHK | HIV gag p24 | 446 |
| HLA-A*1101 | ATIGTAMYK | EBV BRLF1 | 447 |
| HLA-A*1101 | AVDLSHFLK | NEF | 448 |
| HLA-A*1101 | AVFDRKSDAK | EBNA 3B | 449 |
| HLA-A*1101 | IVTDFSVIK | EBNA 3B | 450 |
| HLA-A*1101 | KSMREEYRK | Influenza A MP2 | 451 |
| HLA-A*1101 | NTLEQTVKK | E6 | 452 |
| HLA-A*1101 | RMVLASTTAK | Influenza A MP1 | 453 |
| HLA-A*1101 | SIIPSGPLK | Influenza A MP | 454 |
| HLA-A*1101 | SSCSSCPLSK | EBV LMP-2 | 455 |
| HLA-A*1101 | YVNVNMGLK | HBV core antigen | 456 |
| HLA-A*2301 | QYDPVAALF | pp65 | 457 |
| HLA-A*2402 | AFLPWHRLF | Tyrosinase | 458 |
| HLA-A*2402 | AYACNTSTL | Survivin | 459 |
| HLA-A*2402 | AYAQKIFKI | IE-1 | 460 |
| HLA-A*2402 | AYQGVQQKV | ESAT-6 | 461 |
| HLA-A*2402 | AYSQQTRGL | HCV NS3 | 462 |
| HLA-A*2402 | CYASGWGSI | PSA | 463 |
| HLA-A*2402 | CYTWNQMNL | WT1 | 464 |
| HLA-A*2402 | DYCNVLNKEF | BRLF1 | 465 |
| HLA-A*2402 | DYLNEWGSRF | p-Cadherin | 466 |
| HLA-A*2402 | DYLQYVLQI | BCL-2A1 | 467 |
| HLA-A*2402 | EYCPGGNLF | MELK | 468 |
| HLA-A*2402 | EYILSLEEL | Glypican 3 | 469 |
| HLA-A*2402 | EYLQLVFGI | MAGEA2 | 470 |
| HLA-A*2402 | EYLVSFGVW | HBV core | 471 |
| HLA-A*2402 | EYRALQLHL | Carbonic anhydrase | 472 |
| HLA-A*2402 | EYYELFVNI | DEP DC1 | 473 |
| HLA-A*2402 | GYCTQIGIF | C1orf59 | 474 |
| HLA-A*2402 | IMPKAGLLI | MAGE-A3 | 475 |
| HLA-A*2402 | IYTWIEDHF | FOXM1 | 476 |
| HLA-A*2402 | KLRGEVKQNL | hTOM34p | 477 |
| HLA-A*2402 | KWLISPVKI | HJURP | 478 |
| HLA-A*2402 | KYTSFPWLL | HBV polymerase | 479 |
| HLA-A*2402 | KYYLRVRPLL | KIF20A | 480 |
| HLA-A*2402 | LYQWLGAPV | BGLAP | 481 |
| HLA-A*2402 | NYQPVWLCL | RNF43 | 482 |
| HLA-A*2402 | PYLFWLAAI | EBV LMP2 | 483 |
| HLA-A*2402 | QYDPVAALF | pp65 | 484 |
| HLA-A*2402 | RYCNLEGPPI | LY6K | 485 |
| HLA-A*2402 | RYLKDQQLL | Env | 486 |
| HLA-A*2402 | RYLRDQQLL | Env | 487 |
| HLA-A*2402 | RYNAQCQETI | Midkine | 488 |
| HLA-A*2402 | RYPLTFGW | HIV nef | 489 |
| HLA-A*2402 | RYPLTFGWCF | NEF | 490 |
| HLA-A*2402 | SFHSLHLLF | Tax | 491 |
| HLA-A*2402 | SYRNEIAYL | TTK | 492 |
| HLA-A*2402 | TFPDLESEF | MAGEA3 | 493 |
| HLA-A*2402 | TYACFVSNL | CEA | 494 |
| HLA-A*2402 | TYFSLNNKF | AdV 5 Hexon | 495 |
| HLA-A*2402 | TYGPVFMCL | LMP-2 | 496 |
| HLA-A*2402 | TYGPVFMSL | EBV LMP2 | 497 |
| HLA-A*2402 | TYLPTNASL | HER-2/neu | 498 |
| HLA-A*2402 | VYALPLKML | pp65 | 499 |
| HLA-A*2402 | VYDFAFRDL | HPV16 E6 | 500 |
| HLA-A*2402 | VYFFLPDHL | gp100 | 501 |
| HLA-A*2402 | VYGFVRACL | hTRT | 502 |
| HLA-A*2402 | VYGIRLEHF | Nuf2 | 503 |
| HLA-A*2402 | VYLRVRPLL | KIF20A | 504 |
| HLA-A*2402 | VYYNWQYLL | IL13r | 505 |
| HLA-A*2902 | IACPIVMRY | BRLF1 | 506 |
| HLA-A*2902 | KEKYIDQEEL | HSP90 alpha | 507 |
| HLA-A*2902 | KESTLHLVL | Ubiquitin | 508 |
| HLA-A*2902 | LYNTVATLY | Gag | 509 |
| HLA-A*2902 | SFDPIPIHY | Env | 510 |
| HLA-A*2902 | SFNCRGEFFY | Env | 511 |
| HLA-A*6801 | IVTDFSVIK | EBNA 3B | 512 |
| HLA-A*6801 | TVSGNILTIR | NY-ESO-1 | 513 |
| HLA-B*0702 | APKKPKEPV | VP1 | 514 |
| HLA-B*0702 | APRGVRMAV | CAMEL | 515 |
| HLA-B*0702 | APTKRKGEC | VP1 | 516 |
| HLA-B*0702 | DPRRRSRNL | HCV core | 517 |
| HLA-B*0702 | GPGHKARVL | Gag-pol | 518 |
| HLA-B*0702 | GPLCKADSL | VP1 | 519 |
| HLA-B*0702 | GPRLGVRAT | HCV core | 520 |
| HLA-B*0702 | IPRRIRQGL | HIV-1 env gp120 | 521 |
| HLA-B*0702 | KPTLKEYVL | E7 | 522 |
| HLA-B*0702 | KPYSGTAYNAL | AdV Hexon | 523 |
| HLA-B*0702 | KPYSGTAYNSL | AdV Hexon | 524 |
| HLA-B*0702 | LPVSCPEDL | bZIP factor | 525 |
| HLA-B*0702 | LPVSPRLQL | CEACAM | 526 |
| HLA-B*0702 | LPWHRLFLL | Tyrosinase | 527 |
| HLA-B*0702 | NPTAQSQVM | VP1 | 528 |
| HLA-B*0702 | QPEWFRNVL | Influenza A PB1 | 529 |
| HLA-B*0702 | QPRAPIRPI | EBNA 6 | 530 |
| HLA-B*0702 | RPHERNGFTVL | pp65 | 531 |
| HLA-B*0702 | RPPIFIRRL | EBNA 3A | 532 |
| HLA-B*0702 | RPQGGSRPEFVKL | BMRF1 | 533 |
| HLA-B*0702 | RVRFFFPSL | MAGE-A1 | 534 |
| HLA-B*0702 | SPERKMLPC | VP1 | 535 |
| HLA-B*0702 | SPFFLLLLL | Mucin | 536 |
| HLA-B*0702 | SPIVPSFDM | Influenza A NP | 537 |
| HLA-B*0702 | SPSVDKARAEL | SMCY | 538 |
| HLA-B*0702 | TPGPGVRYPL | NEF | 539 |
| HLA-B*0702 | TPNQRQNVC | P2X5a | 540 |
| HLA-B*0702 | TPRVTGGGAM | pp65 | 541 |
| HLA-B*0702 | VPQYGYLTL | VP1 | 542 |
| HLA-B*0801 | AAKGRGAAL | Neg. Control | 543 |
| HLA-B*0801 | APLLRWVL | HMOX1 | 544 |
| HLA-B*0801 | DIYKRWII | Gag | 545 |
| HLA-B*0801 | EIYKRWII | HIV p24 gag | 546 |
| HLA-B*0801 | ELKRKMIYM | IE-1 | 547 |
| HLA-B*0801 | ELNRKMIYM | IE-1 | 548 |
| HLA-B*0801 | ELRRKMMYM | IE-1 | 549 |
| HLA-B*0801 | FLKEKGGL | HIV-1 nef | 550 |
| HLA-B*0801 | FLRGRAYGL | EBNA 3A | 551 |
| HLA-B*0801 | GEIYKRWII | HIV-1 gag p24 | 552 |
| HLA-B*0801 | GFKQSSKAL | bcr-abl 210 kD fusion protein | 553 |
| HLA-B*0801 | HSKKKCDEL | Polyprotein | 554 |
| HLA-B*0801 | LPHNHTDL | TPR-protein | 555 |
| HLA-B*0801 | QAKWRLQTL | EBV EBNA3A | 556 |
| HLA-B*0801 | QIKVRVDMV | IE-1 | 557 |
| HLA-B*0801 | RAKFKQLL | BZLF1 | 558 |
| HLA-B*0801 | YLKDQQLL | Env | 559 |
| HLA-B*1501 | RLRPGGKKKY | HIV-1 p17 | 560 |
| HLA-B*2705 | ARMILMTHF | Polyprotein | 561 |
| HLA-B*2705 | GRAFVTIGK | Env | 562 |
| HLA-B*2705 | GRFGLATEK | BRAF 27 | 563 |
| HLA-B*2705 | GRFGLATVK | BRAF 27 | 564 |
| HLA-B*2705 | KRWIILGLNK | Gag | 565 |
| HLA-B*2705 | KRWIILGLNKI | Gag | 566 |
| HLA-B*2705 | KRWIILGLNKINR | Gag | 567 |
| HLA-B*2705 | KRWIILGLNKIVR | Gag | 568 |
| HLA-B*2705 | KRWIILGLNKIVR | Gag | 569 |
| HLA-B*2705 | KRWIIMGL | Gag | 570 |
| HLA-B*2705 | KRWIIMGLNK | HIV-1 Gag p24 | 571 |
| HLA-B*2705 | RMFPNAPYL | WT1 | 572 |
| HLA-B*2705 | RRWIQLGLQK | Gag | 573 |
| HLA-B*2705 | SRYWAIRTR | Influenza A NP | 574 |
| HLA-B*3501 | CPNSSIVY | HCV E | 575 |
| HLA-B*3501 | EAAGIGILTY | MART-1 | 576 |
| HLA-B*3501 | EPDLAQCFY | Survivin-3a | 577 |
| HLA-B*3501 | EPLPQGQLTAY | BZLF1 | 578 |
| HLA-B*3501 | EPLSQSQITAY | BZLF1 | 579 |
| HLA-B*3501 | HPNIEEVAL | HCV NS3 | 580 |
| HLA-B*3501 | HPVAEADYFEY | EBNA 1 | 581 |
| HLA-B*3501 | HPVGDADYFEY | EBNA 1 | 582 |
| HLA-B*3501 | HPVGEADYFEY | EBNA 1 | 583 |
| HLA-B*3501 | HPVGQADYFEY | EBNA 1 | 584 |
| HLA-B*3501 | IPSINVHHY | pp65 | 585 |
| HLA-B*3501 | IPYLDGTFY | AdV Hexon | 586 |
| HLA-B*3501 | LPLNVGLPIIGVM | UL138 | 587 |
| HLA-B*3501 | LPSDFFPSV | CP | 588 |
| HLA-B*3501 | MPFATPMEA | NY-ESO-1 | 589 |
| HLA-B*3501 | NPDIVIYQY | HIV-1 RT | 590 |
| HLA-B*3501 | VPLDEDFRKY | RT | 591 |
| HLA-B*3501 | YPLHEQHGM | EBNA 3A | 592 |
| HLA-B*4001 | IEDPPFNSL | EBV LMP2 | 593 |
| HLA-B*4001 | KEKGGLEGL | HIV-1 Nef | 594 |
| HLA-B*4001 | REISVPAEIL | HCV NS5a | 595 |
| HLA-B*5101 | IPFYGKAI | Polyprotein | 596 |
| HLA-B*5101 | LPSDFFPSV | CP | 597 |
| HLA-B*5101 | MPFATPMEA | NY-ESO-1 | 598 |
| HLA-E*0101 | VMAPRTLVL | HLA-A leader sequence peptide | 599 |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least 1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Alcover A, Alarcón B, Di Bartolo V. Cell Biology of T Cell Receptor Expression and Regulation. Annu Rev Immunol. 2018 Apr. 26; 36:103-125.
Altman J D, Davis M M. MHC-Peptide Tetramers to Visualize Antigen-Specific T Cells. Curr Protoc Immunol. 2016 Nov. 1; 115:17.3.1-17.3.44.
Altman J D, Moss P A, Goulder P J, Barouch D H, McHeyzer-Williams M G, Bell J I, McMichael A J, Davis M M. Phenotypic analysis of antigen-specific T lymphocytes. Science. 1996 Oct. 4; 274(5284):94-6.
Andreatta M, Nielsen M. Gapped sequence alignment using artificial neural networks: application to the MHC class I system. Bioinformatics. 2016 Feb. 15; 32(4):511-7.
Beckett D, Kovaleva E, Schatz P J. A minimal peptide substrate in biotin holoenzyme synthetase-catalyzed biotinylation. Protein Sci. 1999 April; 8(4):921-9.
Cho J H, Sprent J. TCR tuning of T cell subsets. Immunol Rev. 2018 May; 283(1):129-137.
Clement M, Ladell K, Ekeruche-Makinde J, Miles J J, Edwards E S, Dolton G, Williams T, Schauenburg A J, Cole D K, Lauder S N, Gallimore A M, Godkin A J, Burrows S R, Price D A, Sewell A K, Wooldridge L. Anti-CD8 antibodies can trigger CD8+ T cell effector function in the absence of TCR engagement and improve peptide-MHCI tetramer staining. J Immunol. 2011 Jul. 15; 187(2):654-63.
Dolton G, Lissina A, Skowera A, Ladell K, Tungatt K, Jones E, Kronenberg-Versteeg D, Akpovwa H, Pentier J M, Holland C J, Godkin A J, Cole D K, Neller M A, Miles J J, Price D A, Peakman M, Sewell A K. Comparison of peptide-major histocompatibility complex tetramers and dextramers for the identification of antigen-specific T cells. Clin Exp Immunol. 2014 July; 177(1):47-63.
Dolton G, Tungatt K, Lloyd A, Bianchi V, Theaker S M, Trimby A, Holland C J, Donia M, Godkin A J, Cole D K, Straten P T, Peakman M, Svane I M, Sewell A K. More tricks with tetramers: a practical guide to staining T cells with peptide-MHC multimers. Immunology. 2015 September; 146(1):11-22.
Greten T F, Korangy F, Neumann G, Wedemeyer H, Schlote K, Heller A, Scheffer S, Pardoll D M, Garbe A I, Schneck J P, Manns M P. Peptide-beta2-microglobulin-MHC fusion molecules bind antigen-specific T cells and can be used for multivalent MHC-Ig complexes. J Immunol Methods. 2002 Dec. 20; 271(1-2):125-35.
Hansen T, Yu Y Y, Fremont D H. Preparation of stable single-chain trimers engineered with peptide, beta2 microglobulin, and MHC heavy chain. Curr Protoc Immunol. 2009 November; Chapter 17:Unit17.5.
Hu Z, Ott P A, Wu C J. Towards personalized, tumour-specific, therapeutic vaccines for cancer. Nat Rev Immunol. 2018 March; 18(3):168-182.
Hung C F, Calizo R, Tsai Y C, He L, Wu T C. A DNA vaccine encoding a single-chain trimer of HLA-A2 linked to human mesothelin peptide generates anti-tumor effects against human mesothelin-expressing tumors. Vaccine. 2007 Jan. 2; 25(1):127-35.
Haryadi R, Ho S, Kok Y J, Pu H X, Zheng L, Pereira N A, Li B, Bi X, Goh L T, Yang Y, Song Z. Optimization of heavy chain and light chain signal peptides for high level expression of therapeutic antibodies in CHO cells. PLoS One. 2015 Feb. 23; 10(2):e0116878.
Kebriaei P, Izsvik Z, Narayanavari S A, Singh H, Ivics Z. Gene Therapy with the Sleeping Beauty Transposon System. Trends Genet. 2017 November; 33(11):852-870.
Khairnar V, Duhan V, Patil A M, Zhou F, Bhat H, Thoens C, Sharma P, Adomati T, Friendrich S K, Bezgovsek J, Dreesen J D, Wennemuth G, Westendorf A M, Zelinskyy G, Dittmer U, Hardt C, Timm J, Göthert J R, Lang P A, Singer B B, Lang K S. CEACAM1 promotes CD8+ T cell responses and improves control of a chronic viral infection. Nat Commun. 2018 Jul. 2; 9(1):2561.
Kowarz E, Loscher D, Marschalek R. Optimized Sleeping Beauty transposons rapidly generate stable transgenic cell lines. Biotechnol J. 2015 April; 10(4):647-53.
Kuo C C, Chiang A W, Shamie I, Samoudi M, Gutierrez J M, Lewis N E. The emerging role of systems biology for engineering protein production in CHO cells. Curr Opin Biotechnol. 2018 June; 51:64-69.
Lowe D B, Bivens C K, Mobley A S, Herrera C E, McCormick A L, Wichner T, Sabnani M K, Wood L M, Weidanz J A. TCR-like antibody drug conjugates mediate killing of tumor cells with low peptide/HLA targets. MAbs. 2017 May/June; 9(4):603-614.
Mátés L, Chuah M K, Belay E, Jerchow B, Manoj N, Acosta-Sanchez A, Grzela D P, Schmitt A, Becker K, Matrai J, Ma L, Samara-Kuko E, Gysemans C, Pryputniewicz D, Miskey C, Fletcher B, VandenDriessche T, Ivics Z, Izsvik Z. Molecular evolution of a novel hyperactive Sleeping Beauty transposase enables robust stable gene transfer in vertebrates. Nat Genet. 2009 June; 41(6):753-61.
Mitaksov V, Truscott S M, Lybarger L, Connolly J M, Hansen T H, Fremont D H. Structural engineering of pMHC reagents for T cell vaccines and diagnostics. Chem Biol. 2007 August; 14(8):909-22.
Schmidt M, Lill J R. MHC class I presented antigens from malignancies: A perspective on analytical characterization & immunogenicity. J Proteomics. 2018 Apr. 24. pii: S1874-3919(18)30181-7.
Soen Y, Chen D S, Kraft D L, Davis M M, Brown P O. Detection and characterization of cellular immune responses using peptide-MHC microarrays. PLoS Biol. 2003 December; 1(3):E65.
White J, Crawford F, Fremont D, Marrack P, Kappler J. Soluble class I MHC with beta2-microglobulin covalently linked peptides: specific binding to a T cell hybridoma. J Immunol. 1999 Mar. 1; 162(5):2671-6.
Wieczorek M, Abualrous E T, Sticht J, Álvaro-Benito M, Stolzenberg S, Noé F, Freund C. Major Histocompatibility Complex (MHC) Class I and MHC Class II Proteins: Conformational Plasticity in Antigen Presentation. Front Immunol. 2017 Mar. 17; 8:292.
Yu Y Y, Netuschil N, Lybarger L, Connolly J M, Hansen T H. Cutting edge: single-chain trimers of MHC class I molecules form stable structures that potently stimulate antigen-specific T cells and B cells. J Immunol. 2002 Apr. 1; 168(7):3145-9.

1. A fusion protein comprising a peptide, a first flexible linker, a β2-microglobulin domain, a second flexible linker, a soluble major histocompatibility complex (MHC) heavy chain and a peptide tag.

2. (canceled)

3. The fusion protein of claim 1, wherein the MHC is at least one of: a human, mouse, rat, hamster, horse, pig, cow, simian, avian, or chimeric MHC, wherein the MHC is Class I MHC, or wherein the MHC does not include a transmembrane sequence.

4. (canceled)

5. (canceled)

6. The fusion protein of claim 1, wherein the peptide tag is selected from wherein the peptide tag is selected from at least one of: a BirA tail, a myc, a FLAG, a glutathione-S-transferase, a His tag, a maltose binding protein, hemagglutinin (HA)-tag, a V5 tag, a T7 tag, a V9 tag, a NusA-tag, a thioredoxin-tag, or a fluorescent protein-tag, a Her2/neu-tag, a CD20-tag, or a GFP-tag.

7. The fusion protein of claim 1, wherein the first, the second, or both the first and second flexible linkers comprise glycine, serine or both glycine and serine and comprise 5 to 40 residues.

8. (canceled)

9. The fusion protein of claim 1, wherein the peptide is selected from at least one of: an immunogenic peptide epitope: a peptide that is 8 to 16 residues long: ABL1; ACPP; Ad5 ElA; AdV 5 Hexon; AdV Hexon; Ag85A; alfa fetoprotein; ASP-2; BA46; BALF4; BAP31; BCL-2; BCL-2A1; BCL-2L1; BCL-X; BCR-ABL; bcr-abl 210 kD fusion protein; Beta-gal; BGLAP; BMI1; BMLF1; BMRF1; BNP; BRAF 27; BRLF1; bZIP factor; BZLF1; C1orf59; CAMEL; Carbonic anhydrase; CB9L2; CD105; CD33; CD59; CEA; CEACAM; Chondromodulin; circadian clock protein PASD1; CP; CPS; cyclin-dependent kinase 4; CYP190; Cytochrome p450; DEP DC1; DLK1; Dutpase; E6; E7; EBNA 1; EBNA 3A; EBNA 3B; EBNA 6; EBV BRLF1; EBV EBNA3A; EBV LMP2; EDDR1; EGFR; EMNA 3A; EMV-1; Enhanced Green Fluorescent Protein; Env; EphA2; Erkl; ESAT-6; EZH2; FAPPa; FLT1; FOLR1; FOXM1; G250; GAD65; Gag; Gag-pol; Glypican 3; gp; gp100; gp33 (C9M); GPC3; H250; HA-1; HA-2; HA-8; HBB; HBsAg; HBV core; HBV polymerase; HBV surface antigen; HCMV IE1; HCMV pp65; HCV core; HCV E; HCV NS3; HCV NS4b; HCV NS5a; HCV NS5B; Heparanase; HER2; HER-2/neu; Histocompatibility antigen 60; HIV gag p24; HIV nef, HIV pol; HIV-1 env gp120; HIV-1 IIIB gp120; HIV-1 nef, HIV-1 p17; HIV-1 RT; HIV-1 US4 gp120; HJURP; HLA-A leader sequence peptide; HLA-A2; HLA-Cw3; Chlamydia trachomatis MOMP; HM1.24; HMMR; HMOX1; HO-1; hPSA; HPV 16 E6; HSP105; HSP90 alpha; hTERT; hTOM34p; hTRT; H-Y; IA-2; IAPP; IDO; IE-1; IE62; IGF; IGRP; IL13r; IL13Ra; Ilr1; Influenza A (PR8) NP; Influenza A MP; Influenza A MP1; Influenza A MP2; Influenza A NP; Influenza A PB1; INFγR; Insulin; Interferon gamma inducible protein (GILT) 30; IRS-2; ITGB8; K8.1; KIF20A; KLK; KLK3; L1; LANA; Large T antigen; Lengsin; Listeria monocytogenes Listeriolysin; LIVIN; LMP-1; LMP-2; LMP-2A; LMP-2A; LY6K; m139; mi41; m145; M164; M2-1; M38; M45; M57; MAGE-1; MAGE-10; MAGE-3; MAGE-4; MAGE-A1; MAGEA2; MAGEA3; MAGE-A5; MAGE-C1; MAGE-C2; MART-1; MC38 adpgk neoantigen; MC-38; MCL-1; MCMV IE1; MELK; Mena; Middle T antigen; Midkine; miHAg SMCY; MOG precursor; MP; MPP11; MS4A1; MSLN; Mtb 16 kDa; Mtb 19 kDa; Mtb85A; mTERT; Mucin; muFAPα; MuLV env; Murine Survivin; MYBPC-2; Mycobacterium bovis antigen 85-A; Mycobacterium tuberculosis ESAT-6; Mycobacterium tuberculosis TB10.4; Myelin basic protein; ND; NEF; NEF; Neu/Her-2/Erbb2 proto-oncoprotein; NG2; Non muscle Myosin-9; Non-muscle Myosin; NP; NP396; NP52; NPM1; NRP-1; NRP-2; NRP-V7 superagonist peptide 8.3 Tg NOD mouse; NS3; Nucleocapsid; Nuf2; NY-ESO-1; OVA; Ovalbumin (subdominant); PiA; β2X5a; p53; p56; P79; PAP-3; PASD1; PAX-5; p-Cadherin; PDGFRbeta; PLAC1; PLAM csp; Plasmodium berghei ANKA acid phosphatase; Plasmodium berghei CSP; Plasmodium CSP; Plasmodium falciparum CSP; Plasmodium falciparum Liver stage antigen; PolA; Polymerase; Polyprotein; pp65; PPE; Pr1; PRAME; Prominin1; PSA; PSCA; PSM β2; PSMA; RGS5; RhoC; RNF43; RSV A strain F protein; RSV NP; RT; S protein; SAA; SART3; Sialidases; SIV gag; SMCY; Spike GP; STEAPI; Surface IgG (sA20-Ig) of A20; Survivin; Survivin-3a; SV40 T antigen; TACE; TARP; TARP 2M; Tax; TB10.3-4; Telomerase; TEM1; tgd057; TGFβ; TNPO3; topII; TPBG; TPR-protein; TRAG; TRP2; Trypanosoma cruzi ASP-2; Trypanosoma cruzi SP; TTK; Tyrosinase; Tyrosine-3-hydroxylase; Ubiquitin; UL105; UL138; UL44; USP9Y; V131; Vaccinia virus Copenhagen Protein G5; Vaccinia virus Host range protein 2; VACCL3_100; VEGFR1; VEGFR2; VEGFR2/KDR fragment 1; Vif; Vinculin; VP1; Vpu; VSV N; West Nile virus NY-99 polyprotein precursor; West Nile Virus polyprotein; WT1; Yellow Fever Virus 17D polyprotein; or ZnT-8.

10. The fusion protein of claim 1, wherein the MHC is selected from at least one of H-2 Db, H-2 Dd; H-2 Dk; H-2 Kb; H-2 Kd; H-2 Kk; H-2 Ld; HLA-A*0101; HLA-A*0201; HLA-A*0301; HLA-A*1101; HLA-A*2301; HLA-A*2402; HLA-A*2902; HLA-A*6801; HLA-B*0702; HLA-B*0801; HLA-B*1501; HLA-B*2705; HLA-B*3501; HLA-B*4001; HLA-B*5101; or HLA-E*0101.

11. The fusion protein of claim 1, wherein the peptide is at least one of SEQ ID NO:1-599.

12. A nucleic acid that expresses a fusion protein comprising a peptide, a first flexible linker, a β2-microglobulin domain, a second flexible linker, a soluble major histocompatibility complex (MHC) heavy chain and a peptide tag.

13. (canceled)

14. The nucleic acid of claim 12, wherein the MHC is at least one of: a human, mouse, rat, hamster, horse, pig, cow, simian, avian, or chimeric MHC, wherein the MHC is Class I MHC, or wherein the MHC does not include a transmembrane sequence.

15. (canceled)

16. (canceled)

17. The nucleic acid of claim 12, wherein the peptide tag is selected from at least one of: a BirA tail, a myc, a FLAG, a glutathione-S-transferase, a His tag, a maltose binding protein, hemagglutinin (HA)-tag, a V5 tag, a T7 tag, a V9 tag, a NusA-tag, a thioredoxin-tag, or a fluorescent protein-tag, a Her2/neu-tag, a CD20-tag, or a GFP-tag.

18. The nucleic acid of claim 12, wherein the first, the second, or both the first and second flexible linkers comprise glycine, serine or both glycine and serine and comprise 5 to 40 residues.

19. (canceled)

20. The nucleic acid of claim 12, wherein the peptide is selected from at least one of: an immunogenic peptide epitope; a peptide that is 8 to 16 residues long; ABL1; ACPP; Ad5 ElA; AdV 5 Hexon; AdV Hexon; Ag85A; alfa fetoprotein; ASP-2; BA46; BALF4; BAP31; BCL-2; BCL-2A1; BCL-2L1; BCL-X; BCR-ABL; bcr-abl 210 kD fusion protein; Beta-gal; BGLAP; BMI1; BMLF1; BMRF1; BNP; BRAF 27; BRLF1; bZIP factor; BZLF1; C1orf59; CAMEL; Carbonic anhydrase; CB9L2; CD105; CD33; CD59; CEA; CEACAM; Chondromodulin; circadian clock protein PASD1; CP; CPS; cyclin-dependent kinase 4; CYP190; Cytochrome p450; DEP DC1; DLK1; Dutpase; E6; E7; EBNA 1; EBNA 3A; EBNA 3B; EBNA 6; EBV BRLF1; EBV EBNA3A; EBV LMP2; EDDR1; EGFR; EMNA 3A; EMV-1; Enhanced Green Fluorescent Protein; Env; EphA2; Erkl; ESAT-6; EZH2; FAPPa; FLT1; FOLR1; FOXM1; G250; GAD65; Gag; Gag-pol; Glypican 3; gp; gp100; gp33 (C9M); GPC3; H250; HA-1; HA-2; HA-8; HBB; HBsAg; HBV core; HBV polymerase; HBV surface antigen; HCMV IE1; HCMV pp65; HCV core; HCV E; HCV NS3; HCV NS4b; HCV NS5a; HCV NS5B; Heparanase; HER2; HER-2/neu; Histocompatibility antigen 60; HIV gag p24; HIV nef, HIV pol; HIV-1 env gp120; HIV-1 IIIB gp120; HIV-1 nef, HIV-1 p17; HIV-1 RT; HIV-1 US4 gp120; HJURP; HLA-A leader sequence peptide; HLA-A2; HLA-Cw3; Chlamydia trachomatis MOMP; HM1.24; HMMR; HMOX1; HO-1; hPSA; HPV 16 E6; HSP105; HSP90 alpha; hTERT; hTOM34p; hTRT; H-Y; IA-2; IAPP; IDO; IE-1; IE62; IGF; IGRP; IL13r; IL13Ra; Ilr1; Influenza A (PR8) NP; Influenza A MP; Influenza A MP1; Influenza A MP2; Influenza A NP; Influenza A PB1; INFγR; Insulin; Interferon gamma inducible protein (GILT) 30; IRS-2; ITGB8; K8.1; KIF20A; KLK; KLK3; L1; LANA; Large T antigen; Lengsin; Listeria monocytogenes Listeriolysin; LIVIN; LMP-1; LMP-2; LMP-2A; LMP-2A; LY6K; m139; m141; m145; M164; M2-1; M38; M45; M57; MAGE-1; MAGE-10; MAGE-3; MAGE-4; MAGE-A1; MAGEA2; MAGEA3; MAGE-A5; MAGE-C1; MAGE-C2; MART-1; MC38 adpgk neoantigen; MC-38; MCL-1; MCMV IE1; MELK; Mena; Middle T antigen; Midkine; miHAg SMCY; MOG precursor; MP; MPP11; MS4A1; MSLN; Mtb 16 kDa; Mtb 19 kDa; Mtb85A; mTERT; Mucin; muFAPα; MuLV env; Murine Survivin; MYBPC-2; Mycobacterium bovis antigen 85-A; Mycobacterium tuberculosis ESAT-6; Mycobacterium tuberculosis TB10.4; Myelin basic protein; ND; NEF; NEF; Neu/Her-2/Erbb2 proto-oncoprotein; NG2; Non muscle Myosin-9; Non-muscle Myosin; NP; NP396; NP52; NPM1; NRP-1; NRP-2; NRP-V7 superagonist peptide 8.3 Tg NOD mouse; NS3; Nucleocapsid; Nuf2; NY-ESO-1; OVA; Ovalbumin (subdominant); P1A; β2X5a; p53; p56; P79; PAP-3; PASD1; PAX-5; p-Cadherin; PDGFRbeta; PLAC1; PLAM csp; Plasmodium berghei ANKA acid phosphatase; Plasmodium berghei CSP; Plasmodium CSP; Plasmodium falciparum CSP; Plasmodium falciparum Liver stage antigen; PolA; Polymerase; Polyprotein; pp65; PPE; Pr1; PRAME; Prominin1; PSA; PSCA; PSM β2; PSMA; RGS5; RhoC; RNF43; RSV A strain F protein; RSV NP; RT; S protein; SAA; SART3; Sialidases; SIV gag; SMCY; Spike GP; STEAPI; Surface IgG (sA20-Ig) of A20; Survivin; Survivin-3a; SV40 T antigen; TACE; TARP; TARP 2M; Tax; TB10.3-4; Telomerase; TEM1; tgd057; TGFβ; TNPO3; topII; TPBG; TPR-protein; TRAG; TRP2; Trypanosoma cruzi ASP-2; Trypanosoma cruzi SP; TTK; Tyrosinase; Tyrosine-3-hydroxylase; Ubiquitin; UL105; UL138; UL44; USP9Y; V131; Vaccinia virus Copenhagen Protein G5; Vaccinia virus Host range protein 2; VACCL3_100; VEGFR1; VEGFR2; VEGFR2/KDR fragment 1; Vif; Vinculin; VP1; Vpu; VSV N; West Nile virus NY-99 polyprotein precursor; West Nile Virus polyprotein; WT1; Yellow Fever Virus 17D polyprotein; or ZnT-8.

21. The nucleic acid of claim 12, wherein the MHC is selected from at least one of H-2 Db, H-2 Dd; H-2 Dk; H-2 Kb; H-2 Kd; H-2 Kk; H-2 Ld; HLA-A*0101; HLA-A*0201; HLA-A*0301; HLA-A*1101; HLA-A*2301; HLA-A*2402; HLA-A*2902; HLA-A*6801; HLA-B*0702; HLA-B*0801; HLA-B*1501; HLA-B*2705; HLA-B*3501; HLA-B*4001; HLA-B*5101; or HLA-E*0101.

22. The nucleic acid of claim 12, wherein the peptide is SEQ ID NO:1-599.

23. A method of making a soluble eukaryotic-derived peptide/MHC complex comprising:
expressing in a cell a fusion protein comprising a peptide, a first flexible linker, a β2-microglobulin domain, a second flexible linker, a soluble major histocompatibility complex (MHC) heavy chain and a peptide tag.

24. The method of claim 23, further comprising isolating the fusion protein from a supernatant.

25. The method of claim 23, further comprising forming dimers, trimers, tetramers, or multimers of the fusion protein by mixing the fusion protein with one or more agents that bind to two or more fusion proteins.

26. The method of claim 25, wherein the agent is selected from an antibody, a cross-linking agent, a ligase, an avidin, a streptavidin, a Protein A, or a J-chain.

27. (canceled)

28. (canceled)

29. (canceled)

30. (canceled)

31. (canceled)

32. (canceled)

33. (canceled)

34. A cell line expressing a fusion protein comprising a fusion protein comprising a peptide, a first flexible linker, a β2-microglobulin domain, a second flexible linker, a soluble major histocompatibility complex (MHC) heavy chain and a peptide tag.

35. The cell line of claim 34, wherein the fusion protein is integrated into the genome by co-transfecting a fusion protein expressing vector with a transposase vector that expresses a transposase and wherein the fusion protein expressing vector, the transposase vector, or both further comprise a selectable marker.

36. (canceled)

37. (canceled)

38. (canceled)

39. (canceled)

40. (canceled)

41. (canceled)

42. (canceled)
